(19) United States
(12) Patent Application Publication
(10) Pub. No.: US 2014/0186372 A1
(43) Pub. Date: Jul. 3, 2014

(54) COMPOSITIONS TARGETING PKC-THETA AND USES AND METHODS OF TREATING PKC-THETA PATHOLOGIES, ADVERSE IMMUNE RESPONSES AND DISEASES

(71) Applicants: Amnon Altman, La Jolla, CA (US); Kok-Fai Kong, San Diego, CA (US)

(72) Inventors: Amnon Altman, La Jolla, CA (US); Kok-Fai Kong, San Diego, CA (US)

(73) Assignee: LA JOLLA INSTITUTE FOR ALLERGY AND IMMUNOLOGY, San Diego, CA (US)

(21) Appl. No.: 14/125,298

(22) PCT Filed: Jun. 15, 2012
(86) PCT No.: PCT/US12/42722

§ 371 Date: Mar. 7, 2014

Related U.S. Application Data
(60) Provisional application No. 61/497,884, filed on Jun. 16, 2011.

(51) Int. Cl. G01N 33/573

(52) U.S. Cl. CPC G01N 33/573
USPC 424/172.1; 435/7.24; 435/375; 435/377; 514/21.4

(57) ABSTRACT

The invention relates to compositions, methods and uses of inhibitors of binding between PKCθ and CD28, and modulating an undesirable or aberrant immune response, disorder or disease, an inflammatory response, disorder or disease, inflammation or an autoimmune response, disorder or disease. Compositions include inhibitors of binding between PKCθ and CD28, which include, among others, PKCθ, CD28 and Lck sequences, subsequences, variants and modified forms, and polymorphisms.

### RELATED APPLICATIONS

This application is a U.S. National Phase of International Application No. PCT/US2012/04272, filed Jun. 15, 2012, which designated the U.S. and that International Application was published under PCT Article 21(2) in English, and claims priority to U.S. Provisional Application No. 61/497,884, filed Jun. 16, 2011, all of which applications are incorporated herein by reference in their entirety.

### GOVERNMENT SUPPORT

This work was supported in part by grant CA035299 from the National Institutes of Health. The government has certain rights in the invention.

### INTRODUCTION

The induction of an immune response depends on effective communication between antigen-specific T cells and antigen presenting cells (APCs). When a T cell expressing a cognate T cell receptor (TCR) encounters an activated APC, both cells actively redistribute their receptors and ligands to the interface, creating a platform for effective signaling, known as the immunological synapse (IS). At steady state, the mature IS is composed of concentric rings with a central core (cSMAC) containing clusters of TCR and costimulatory molecules, and a peripheral ring (pSMAC) of adhesion molecules (Dustin, M. L. Immunity 30, 482-492 (2009)). The engagement of these surface molecules triggers signaling cascades resulting in the recruitment of intracellular proteins, including kinases, adapter and cytoskeletal proteins to the IS (Dustin, M. L. et al. Cold Spring Harb Perspect Biol 2, a002311 (2010)). One of the most prominent proteins to be specifically recruited to the IS following antigen stimulation is protein kinase C-θ (PKCθ), whose localization is limited to the cSMAC (Monks, C. R. et al. Nature 385, 83-86 (1997), (Monks, C. R. et al., Nature 395, 82-86 (1998)).

PKCθ is a member of the novel, Ca²⁺-independent PKC subfamily expressed predominantly in T cells (but also in muscle), which plays important and non-redundant roles in T cell activation and survival (but not in T cell development) (Pfeifhofer, C. et al., J Exp Med 197, 1525-1535 (2003); Sun, Z. et al., Nature 404, 402 (2000); Hayashi, K. et al., Pharmacol Res 55, 537-544 (2007)), reflecting its unique ability to activate the transcription factors NF-κB, AP-1 and, more recently, also NFAT (Pfeifhofer, C. et al., J Exp Med 197, 1525-1535 (2003); Coudronniere, N. et al. Proc Natl Acad Sci USA 97, 3394-3399 (2000); Baier-Bitterlich, G. et al. Mol Cell Biol 16, 1842-1850 (1996); Altman, A. et al. Eur J Immunol 34, 2001-2011 (2004); Lin, X. et al. Mol. Cell. Biol. 20, 2933-2940 (2000); Manicassamy, S. et al. J Mol Biol 355, 347-359 (2006)). Studies using PKCθ-deficient (PKCθ^{-/-}) mice have characterized the importance of PKCθ in different disease models and, surprisingly, have revealed that its requirement in different forms of immunity is quite selective and not absolute. Thus, Th2 responses against allergens or helminth infection (Marsland, B. et al. J Exp Med 200, 181-189 (2004), Salek-Ardakani, S. et al. J Immunol 173, 6440-6447 (2004)) and Th17-mediated autoimmune diseases (Salek-Ardakani, S. et al. J Immunol 175, 7635-7641 (2005); Anderson, K. et al. Autoimmunity 39, 469-478 (2006); Tan, S. L. et al. J Immunol 176, 2872-2879 (2006)) require PKCθ. In contrast, there was no defect in the development of Th1 immune responses against intracellular pathogens such as Leishmania major (Marsland, B. et al. J Exp Med 200, 181-189 (2004)), as well as antiviral effector and memory cytotoxic T lymphocyte responses (Berg-Brown, N. N. et al. J Exp Med 199, 743-752 (2004); Giannoni, F. et al. J Virol 178, 3466-3473 (2007); Marsland, B. J. et al. Proc Natl Acad Sci USA 102, 14374-14379 (2005); Marsland, B. J. et al. J Immunol 178, 3466-3473). Consistent with these in vivo findings, PKCθ^{-/-} CD4 T cells display impaired in vitro differentiation into the Th2 and Th17 lineages, while Th1 differentiation is only moderately reduced (Marsland, B. et al. J Exp Med 200, 181-189 (2004), Salek-Ardakani, S. et al. J Immunol 173, 6440-6447 (2004); Salek-Ardakani, S. et al. J Immunol 175, 7635-7641 (2005)). More recently, PKCθ was found to be required for allograft rejection and graft vs. host (GvH) disease, but not for graft vs. leukemia (GvL) response in mice (Valenzuela, J. O. et al. J Clin Invest 119, 3774-3786 (2009)).

PKCθ is a mediator of TCR/CD28 cosignaling in T cells, and is required for T cell activation and survival. Although the catalytic activity of PKCθ is undoubtedly required for its downstream signaling functions, its proper localization in defined plasma membrane domains, i.e., the IS (Monks, C. R. et al. Nature 385, 83-86 (1997)) and lipid rafts (Bi, K. et al. Nat Immunol 2, 556-563 (2001)), which is mediated by its regulatory region, is also critical. However, despite some circumstantial evidence (Monks, C. R. et al. Nature 395, 82-86 (1998); Huang, J. et al. Proc Natl Acad Sci USA 99, 9369-9373 (2002)), the precise relationship between the cSMAC localization of PKCθ and its non-redundant functions, and whether the former is required for the latter, has not been known, nor have the structural determinants that dictate this unique localization been identified.

### SUMMARY

Antigen-induced localization of PKCθ to the IS and, more specifically, to the cSMAC is well established (Monks, C. R. et al. Nature 385, 83-86 (1997); Monks, C. R. et al. Nature 395, 82-86 (1998)), but the molecular basis for this highly selective localization is not clear, nor is it known whether it is required for the signaling functions of PKCθ in T cells. As disclosed herein, the identification and characterization of the hinge region of PKCθ, known as the V3 domain, as playing a critical role in determining its IS/cSMAC localization via binding to CD28 and, consequently, dictating its signaling from the IS. Fine mapping further revealed an evolutionarily conserved proline-rich (PR) motif that is required for CD28 association, cSMAC localization and PKCθ-mediated functions. The isolated V3 domain of PKCθ behaved as a decoy in a dominant negative fashion to block PKCθ-dependent functions, including Th2- and Th17, but not Th1, differentiation and inflammation. These findings implicate a unique signaling mode of CD28, and establish the molecular basis for the specialized localization and function of PKCθ in antigen-stimulated T cells.

In accordance with the invention, there are provided methods of decreasing, reducing, inhibiting, suppressing, limiting or controlling an undesirable or aberrant immune response, immune disorder, inflammatory response, or inflammation in a subject; and methods of decreasing, reducing, inhibiting, suppressing, limiting or controlling an autoimmune response, disorder or disease in a subject. In one embodiment, a method includes administering an inhibitor of binding between protein kinase C (PKC) theta (PKCθ) and CD28 to a subject in an amount to decrease, reduce, inhibit, suppress, limit or control the undesirable or aberrant immune response, immune disorder, inflammatory response, or inflammation, or to decrease, reduce, inhibit, suppress, limit or control an autoimmune response, disorder or disease, in the subject.

In accordance with the invention, there are also provided methods of reducing, inhibiting, suppressing, or limiting binding of protein kinase C (PKC) theta (PKCθ) to CD28. In one embodiment, a method includes contacting CD28 binding region of PKCθ with an inhibitor that binds to the CD28 binding region of PKCθ thereby reducing, inhibiting, suppressing, or limiting binding of PKCθ to CD28.

In accordance with the invention, there are further provided methods of increasing, inducing, stimulating, or promoting regulatory T cell (Tregs) differentiation or function. In one embodiment, a method includes administering an inhibitor of binding between protein kinase C (PKC) theta (PKCθ) and CD28 in an amount effective for increasing, inducing, stimulating, or promoting regulatory T cell differentiation or function.

In accordance with the invention, there are additionally provided protein kinase C (PKC) theta (PKCθ) sequences and compositions, including pharmaceutical compositions that include or consist of a protein kinase C (PKC) theta (PKCθ) sequence, such as a PKCθ sequence that inhibits or reduces binding between PKCθ and CD28. In various embodiments, a protein kinase C (PKC) theta (PKCθ) sequence includes or consists of a ARPPCLPTP sequence, a substitution of an amino acid in a ARPPCLPTP sequence, a sequence motif set forth in Table 1, or a substitution of an amino acid in a sequence motif set forth in Table 1 (e.g., ARPPCLPTP, ARLPCVPAP, ARLPCVPAS, AKLPHAPAP, AKPPYVPGP, or TRLPYLPTP, etc.). In particular aspects, the sequence has a length from 9 to about 700 amino acids, wherein the 9 to about 700 amino acid sequence includes all or portion of a PKCθ amino acid sequence, or does not include all or a portion of a PKCθ amino acid sequence. Exemplary sequences are about 9-20, 20-30, 30-40, 40-50, 50-75, 75-100, 100-150, 150-200, 200-250, 250-300, 300-350, 350-400, 400-500, 500-600 or 600-700 amino acid residues in length.

In accordance with the invention, there are moreover provided methods for screening and/or identifying an agent that decreases, reduces or inhibits interaction of PKCθ with CD28. In one embodiment, a method includes contacting PKCθ with CD28 under conditions allowing binding between PKCθ and CD28 in the presence a test agent; and determining if the test agent inhibits or reduces binding between PKCθ and CD28, thereby screening for and/or identifying an agent that decreases, reduces or inhibits interaction of PKCθ with CD28. In particular aspects, such screening and/or identifying methods can be used to determine if the agent is a candidate agent for decreasing, reducing, inhibiting, suppressing, limiting or controlling an undesirable or aberrant immune response, immune disorder, inflammatory response, or inflammation, an autoimmune response, disorder or disease, or decreasing, reducing, inhibiting, suppressing, limiting or controlling graft vs. host disease (GVHD).

### DESCRIPTION OF THE DRAWINGS

FIGS. 1a-1h show that the V3 domain of PKCθ is required for its IS/cSMAC localization, downstream signaling and for NFAT activation. (a) PKCθ^{-/-} CD4⁺ T cells from OT-II Tg mice were infected with retrovirus expressing GFP-tagged WT PKCθ, PKCθ-ΔV3, or PKCθ/δV3 (green). Cells were mixed at a 1:1 ratio with CMAC (blue)-labeled APC pre-incubated with or without Ova peptide. Conjugates were fixed and stained with anti-talin plus a secondary Alexa 647-coupled antibody (red). (b) Quantitative analysis of the results shown in (a). Localization of PKCθ in the IS/cSMAC was analyzed in 30-50 T-APC conjugates. Only cell conjugates that had reorganized their talin and that had visibly detectable levels of the protein of interest were analyzed for translocation. ** p<0.05. (c) MCC-specific T hybridoma cells were cotransfected with empty pEF vector or the indicated Xpress-tagged PKCθ vectors, together with CD28-response element-luciferase (Luc) reporter (RE/AP) and a β-Gal reporter plasmid. Cells were incubated with I-E^{k}- and B7-1-expressing DCEK fibroblasts in the absence or presence of MCC peptide for 6 h. Normalized Luc activity was determined in triplicates. Expression levels of the transfected proteins, revealed by anti-Xpress immunoblotting, are shown at the bottom. ** p<0.05. (d-g) BM cells from PKCθ^{-/-} mice were transduced with empty pMIG-vector alone, WT PKCθ, or PKCθ/δV3 and used to reconstitute Rag^{-/-} mice. Sorted GFP⁺ CD4⁺ T cells were left unstimulated or stimulated overnight with anti-CD3 plus anti-CD28 mAbs to determine the expression of CD69 (d) or CD25 and PKCθ (e) or for 48 h to determine the production of; (f) IL-2; and (g) proliferation. (h) Importance of the PKCθ V3 domain for NFAT activation. MCC-specific T hybridoma cells were cotransfected with empty pEF vector, WT PKCθ, PKCθ-ΔV3, or PKCθ/δV3, together with NFAT luciferase reporter and β-Gal reporter plasmids. Cells were cultured with DECK fibroblasts expressing I-E^{k} and B7-1 in the absence or presence of MCC peptide for 6 hrs. ** p<0.05. Data shown is for 2 studies.

FIGS. 2a-2f show that the PKCθ V3 domain interacts with CD28. (a) Schematic representation of PKCθ mutants. (b) Jurkat E6.1 cells transfected with the indicated PKCθ or PKCδ vectors were stimulated with anti-CD3/CD28 mAbs for 5 min. (c-e) PKCθ^{-/-} CD4⁺ T cells were infected with retrovirus expressing empty pMIG vector, WT PKCθ, or PKCθ-ΔV3 (c); WT PKCθ, PKCθ/δV3, or WT PKCδ (d); empty pMIG-vector or Myc-tagged V3 of PKCθ (e). Cells were harvested and restimulated with CD3+CD28 mAbs for 5 min. Cell lysates were prepared, immunoprecipitated with anti-CD28 mAb, resolved by SDS-PAGE, and immunoblotted with the indicated Abs. (f) Jurkat T cells cotransfected with Myc-tagged PKCθ-V3 plus CFP-tagged CD28 were mixed with SEE-pulsed Raji B cells at a 1:1 ratio. Conjugates were fixed and stained with a rabbit anti-Myc Ab plus a secondary Alexa 488-coupled antibody. Data shown is from four studies.

FIGS. 3a-3e show that a PR motif in the V3 domain of PKCθ determines its IS localization, interaction with CD28 and NFAT activation. (a) CD4⁺ T cells from OT-II Tg mice were infected with retrovirus expressing GFP-tagged WT PKCθ, PKCδ with an insertion of the PR motif (PKCδ+θPR), or WT PKCδ (green). Cells were mixed at a 1:1 ratio with CMAC (blue)-labeled APC preincubated with or without Ova peptide. Conjugates were fixed, stained and analyzed as in FIG. 1a. (b) Quantitative analysis of the results shown in (a) was performed as in FIG. 1a. ** p<0.05. (c) PKCθ^{-/-} CD4⁺ T cells were infected with retrovirus expressing WT PKCθ, PKCδ+θPR, or WT PKCδ. Cells were harvested, restimulated with CD3+CD28 mAbs for 5 min (left panel) or left unstimulated (right panel). Asterisk in the right panel indicates the position of the immunoprecipitating antibody heavy chain. (d) MCC-specific T hybridoma cells were cotransfected with the same vectors as in (a) together with an RE/AP-Luc and a β-Gal reporter plasmids. Cells were stimulated as in FIG. 1c, and Normalized Luc activity was determined in triplicates. ** p<0.05 (e) MCC-specific T hybridoma cells were cotransfected with empty pEF vector, WT PKCθ, PKCδ with PRR, or WT PKCδ, together with NFAT luciferase reporter and β-Gal reporter plasmid. Cells were incubated with fibroblasts that express I E* and B7-1 in the absence or presence of MCC peptide for 6 hrs. Normalized luciferase activity was determined in triplicates. ** p<0.05.

FIGS. 4a-4e show the importance of the PxxP motif in the V3 domain of PKCθ for IS localization and CD28 interaction and NFAT signaling. (a) PKCθ^{-/-} OT-II CD4⁺ T cells were infected with retrovirus expressing GFP-tagged PKCθ, or PKCθ-GFP fusion vectors containing mutations at P330/6A, P331/4A, or all four proine residues (4P-A) (green). Cells were fixed, stained and analyzed as in FIG. 1a. (b) Quantitative analysis of the results shown in (a) was performed as in FIG. 1a. ** p<0.05. (c) PKCθ^{-/-} CD4⁺ T cells were infected with retrovirus expressing WT PKCθ, or P330/6A, P331/4A or P330/1/4/6 (4P-A). Cells were harvested and restimulated with CD3+CD28 mAbs for 5 min (d) MCC-specific T hybridoma cells were co-transfected with empty pEF vector or the indicated PKCθ mutants together with RE/AP β-Gal reporter plasmids. Cells were stimulated, and normalized Luc activity was determined as in FIG. 1c. ** p<0.05; (e) MCC-specific T hybridoma cells were cotransfected with each of the indicated PKCθ vectors together with NFAT luciferase reporter and a β-Gal reporter plasmid. Cells were incubated with fibroblasts that express I-EK and B7-1 in the absence or presence of MCC peptides for 6 hrs. Normalized luciferase activity was determined in triplicates. ** p<0.05.

FIGS. 5a-5d show that the importance of the PxxP motif in PKCθ-mediated signaling. (a-d) PKCθ^{-/-} BM cells were transduced with retrovirus expressing empty pMIG vector, or the indicated PKCθ vectors used to reconstitute Rag^{-/-} mice. Sorted GFP⁺ CD4⁺ T cells were left unstimulated or stimulated overnight with anti-CD3 plus anti-CD28 mAbs to determine the expression of CD69 (a) or CD25 and PKCθ (b); or for 48 h to determine the production of IL-2 (c), and proliferation (d).

FIGS. 6a-6e show that the V3 domain interferes with PKCθ-mediated signaling, T cell differentiation and NFAT activation. (a) OT-II CD4⁺ T cells were infected with retrovirus expressing Myc-tagged V3 domain, V3 mutated at P330/1/4/6A (V3-4PA), or PR motif-deleted V3 (V3-ΔPR). Infected cells (green) were harvested, stimulated, and fixed. Conjugates were stained with anti-Myc plus a secondary Alexa 555-coupled antibody (orange), and anti-PKCθ plus a secondary Alexa 647-coupled antibody (red). Cells were analyzed as in FIG. 1a. (b) Quantitative analysis of the results shown in (a) from 30-50 T-APC conjugates. ** p<0.05. (c) MCC-specific T hybridoma cells were cotransfected with indicated vector, together with RE/AP-Luc and β-Gal reporter plasmids. Cells were stimulated and analyzed as in FIG. 1c. (d) Naïve CD4⁺ T cells from B6 mice stimulated with anti-CD3 plus anti-CD28 mAbs and differentiated in vitro under Th1-, Th2- or Th17-polarizing conditions were retrovirally transduced with empty pMIG vector, or with the indicated PKCθ V3 vectors. Cytokine-producing cells were analyzed by intracellular staining 8 h after restimulation. Right panels represent cumulative data showing percentage of cytokine-producing cells by intracellular staining. (e) MCC-specific T hybridoma cells were cotransfected with the indicated plasmids, together with NFAT luciferase reporter and a β-Gal reporter plasmid. Cells were incubated with fibroblasts that express I-E^{K} and B7-1 in the absence or presence of MCC peptide for 6 hrs. Normalized luciferase activity was determined in triplicates.

FIGS. 7a-7e show that V3 inhibits Th2-, but not Th1-mediated, lung inflammation. (a-d) OT-II CD4⁺ T cells stimulated with anti-CD3 plus anti-CD28 mAbs and differentiated in vitro under Th2 (a-c) or Th1 (d, e)-polarizing conditions were retrovitrally transduced with the same PKCθ V3 vectors as in FIG. 6. Sorted GFP⁺ populations were adoptively transferred into naïve B6 mice, which were challenged with aerosolized Ova for 3 consecutive days. BAL fluid were obtained 1 d post-challenge and analyzed for total mononuclear cell infiltration (a, d) and cytokine expression using IL-4 (b), IL-5 (c) and IFN-γ (e) ELISA.

FIGS. 8a-8c show intracellular uptake of the R9-PR and R9-Scr peptides by primary CD4+ T cells. (a) Sequence of cell-permeable peptides. R9-PR refers to the specific peptide based on the PKC-theta sequence critical for CD28 interaction (underlined), with the critical proline-rich motif (PCVP) in bold, and with 9 arginine residues added at the N-termunus to render the peptide cell-permeable. R9-Scr refers to a similar peptide in which the underlined sequence has been scrambled, and serves as a negative control. (b) Primary mouse CD4+ spleen T cells were purified and incubated with the same specific (left panel) or control (right panel) peptides, which have been conjugated to FITC, for 30 min at 37 degrees C. Cells were treated with trypsin for an additional 10 min to remove externally bound peptides. The figure shows intracellular uptake of the fluorescent peptides added to the cells at 2.5 μM (thin lines) or 5 μM (thick line) analyzed by flow cytometry. Shaded histogram shows background staining in cells not incubated with FITC-peptide. (c) Jurkat T cells were subjected to treatment with FITC-peptides as in (b). The cells were fixed and stained with mouse anti-human CD4+ AlexaFluor 555-conjugated secondary anti-mouse antibody (red), and with DAPI (a nuclear stain in blue). Midsection confocal images demonstrate the uptake of the FITC-peptides (green), where the cell membrane is outlined by the red dots (representing surface CD4).

FIG. 9 shows that R9-PR, but not control R9-Scr peptide can disrupt the PKCtheta-CD28 interaction. Jurkat T cells were incubated with specific or control peptide 30 min, 37 degrees C. at the indicated concentrations in μM. Cells were left untreated (left lane) or stimulated with anti-CD3 plus anti-CD28 for 5 min at 37 degrees C. before cell lysis. Cell lysates were immunoprecipitated with anti-CD28 and the IPs were blotted with anti-PKCtheta antibody (top panel), or anti-CD28 (middle panel). Top panel shows that the specific peptide inhibited the interaction of PKCtheta with CD28 (lanes 2, 3) relative to the control peptide (lanes 4, 5). Bottom panel represents samples of whole cell lysates from each group immunoblotted with anti-PKCtheta, showing that similar amounts of PKCtheta were present in all groups. This serves as a loading control for the top panel.

FIG. 10 shows that the V3 domain interferes with PKCθ-mediated differentiation of Th9 cells. Naïve CD4+ T cells from B6 mice stimulated with anti-CD3 plus anti-CD28 mAbs and differentiated in vitro under Th9-polarizing conditions (IL-4+ TGFβ) were retrovirally transduced with empty pMIG vector, or with the indicated PKCθ V3 vectors. Transduced (GFP+) cells were sorted and IL-9-producing cells were analyzed by intracellular staining 8 h after restimulation. Cumulative data showing percentage of cytokine-producing. V3-4PA refers to mutation of 4 Pro residues in the Pro-rich motif to Ala and V3-DPR refers to deletion of the Pro-rich motif (ARPPCLPTP).

FIG. 11 shows that the V3 domain of PKCθ promotes differentiation of iTregs (FoxP3+). Naïve CD4+ T cells from B6 mice stimulated with anti-CD3 plus anti-CD28 mAbs and differentiated in vitro under Treg-polarizing conditions (IL-2+ TGFβ) were retrovirally transduced with empty pMIG vector, or with the indicated PKCθ V3 vectors. Transduced (GFP+) cells were sorted and FoxP3+ cells were analyzed by intracellular staining.

FIG. 12 Interaction between CD28 and the V3 of PKCθ is Lck-dependent. PKCθ-Lck-CD28 association in Lck-deficient (JCam1.6) Jurkat cells cotransfected with Myc-tagged PKCθ-V3 plus WT Lck or its indicated mutants, Transfected cells were stimulated with anti-CD3 and anti-CD28 for 5 min, immunoprecipitated with an anti-Myc Ab, and immunoblotted for Lck and endogenous CD28. Data are from three experiments.

### DETAILED DESCRIPTION

The invention is based, at least in part, on the identification of a region of protein kinase C (PKC) theta (PKCθ) that mediates localization to the immunological synapse (IS), and signaling function of PKCθ. In particular, as disclosed herein, a hinge region of PKCθ, known as the V3 domain, is identified and characterized as playing a critical role in determining IS/cSMAC localization via binding to CD28 and, consequently, dictating its signaling from the IS. As also disclosed herein, a conserved proline-rich (PR) sequence motif, denoted as a PXXP motif in the V3 domain, is required for CD28 association, cSMAC localization and PKCθ-mediated functions. An isolated V3 domain of PKCθ blocked PKCθ-dependent functions, such as Th2- and Th17 mediated differentiation and inflammation and NFAT activation, but not Th1, differentiation and inflammation. PKCθ sequences, compositions and methods, and uses of inhibitors of PKCθ binding to CD28 are therefore useful for modulating PKCθ effector functions and activities.

Accordingly, the invention provides, inter alia, PKCθ polypeptides, subsequences and inhibitors of binding between PKCθ and CD28, compositions thereof, and methods and uses of PKCθ polypeptides, subsequences and inhibitors of binding between PKCθ and CD28. Methods and uses include, for example, modulation and/or treatment of undesirable or aberrant immune responses, immune disorders, inflammatory responses, and inflammation. Methods and uses also include, for example, modulation and/or treatment of autoimmune responses, disorders and diseases. Methods and uses further include, for example, modulation (e.g., reducing, inhibiting, suppressing, limiting; or increasing, inducing, stimulating, or promoting) of binding of protein kinase C (PKC) theta (PKCθ) to CD28. Methods and uses additionally include, for example, modulation (e.g., increasing, inducing, stimulating, promoting) of regulatory T cell (Tregs) differentiation or function. Methods and uses moreover include, for example, in a subject, such as a mammal (e.g., human).

Compositions, methods and uses of the invention include PKCθ, CD28 and Lck polypeptides, and subsequences and fragments of PKCθ, CD28 and Lck polypeptides. In one embodiment, a PKCθ polypeptide subsequence or fragment is characterized as including or consisting of a subsequence of PKCθ (e.g., not full length PKCθ) which inhibits or reduces PKCθ binding to CD28 (in solution, in solid phase, in vitro, ex vivo, or in vivo). In another embodiment, a CD28 polypeptide subsequence or fragment is characterized as including or consisting of a subsequence of CD28 (e.g., not full length CD28 which inhibits or reduces PKCθ binding to CD28 (in solution, in solid phase, in vitro, ex vivo, or in vivo). In a further embodiment, a Lck polypeptide subsequence or fragment is characterized as including or consisting of a subsequence of Lck (e.g., not full length Lck, such as an SH2 and/or SH3 domain which inhibits or reduces PKCθ binding to CD28, in solution, in solid phase, in vitro, ex vivo, or in vivo). Such PKCθ, CD28 and Lck polypeptide sequences, subsequences/fragments, modified forms and variants and polymorphisms as set forth herein, are also included as invention compositions, methods and uses.

In further embodiments, a subsequence or fragment of a PKCθ or CD28 or Lck polypeptide includes or consists of one or more amino acids less than full length PKCθ, CD28 and Lck polypeptides, respectively, and optionally that inhibit or reduce binding of PKCθ to CD28. The term “subsequence” or “fragment” means a portion of the full length molecule. A subsequence of a polypeptide sequence, such as a PKCθ or CD28 or Lck sequence, has one or more amino acids less than a full length PKCθ or CD28 or Lck (e.g. one or more internal or terminal amino acid deletions from either amino or carboxy-termini). Subsequences therefore can be any length up to the full length native molecule, provided said length is at least one amino acid less than full length native molecule.

Subsequences can vary in size, for example, from a polypeptide as small as an epitope capable of binding an antibody (i.e., about five to about eight amino acids) up to a polypeptide that is one amino acid less than the entire length of a reference polypeptide such as PKCθ, CD28 or Lck. In various embodiments, a polypeptide subsequence is characterized as including or consisting of a PKCθ sequence with less than 705 amino acids in length identical to PKCθ, a CD28 sequence with less than 220 amino acids in length identical to CD28, and a Lck sequence with less than 509 amino acids in length identical to Lck. Non-limiting exemplary subsequences less than full length PKCθ sequence include, for example, a subsequence from about 5 to 10, 10 to 20, 20 to 30, 30 to 50, 50 to 100, 100 to 150, 150 to 200, 200 to 300, or 300 to 400, 400-500, 500-600, or 600-705 amino acids in length. Non-limiting exemplary subsequences less than full length CD28 sequence include, for example, a subsequence from about 5 to 10, 10 to 20, 20 to 30, 30 to 50, 50 to 100, 100 to 150, 150 to 200, 200 to 220 amino acids in length. Non-limiting exemplary subsequences less than full length Lck sequence include, for example, a subsequence from about 5 to 10, 10 to 20, 20 to 30, 30 to 50, 50 to 100, 100 to 150, 150 to 200, 200 to 300, or 300 to 400, or 400-510 amino acids in length, e.g., that includes or consists of an SH2 and/or SH3 domain. As used herein, subsequences may also include or consist of one or more amino acid additions or deletions, wherein the subsequence does not comprise full length native/wild type PKCθ, CD28 or Lck sequence. Accordingly, total subsequence lengths can be greater than the length of full length native/wild type PKCθ, CD28 or Lck polypeptide, for example, where a PKCθ, CD28 or Lck subsequence is fused or forms a chimera with another polypeptide.

PKCθ, CD28 and Lck polypeptides include mammalian sequences, such as human, gorilla, chimpanzee, orangutan, or macaque PKCθ, CD28 or Lck sequences. Non-limiting exemplary full length mammalian PKCθ polypeptide sequences showing the PXXP motif (underlined), as disclosed herein, are as follows (SEQ ID NOs:1-5):

| Human PKC-theta | |
|---|---|
| 1 mspflrigls nfdcgscqsc qgeavnpyca vlvkeyvese ngqmyiqkkp | 61 |
| tmyppwdstf | |
| dahinkgrvm qiivkgknvd lisettvely slaercrknn gkteiwlelk | 121 |
| pqgrmlmnar | |
| yflemsdtkd mnefetegff alhqrrgaik qakvhhvkch teftatffpqp | 181 |
| fcsvchefv | |
| wglnkqgyqc rqcnaaihkk cidkviakct gsainsretm | 241 |
| fhkerfkidm phrfkvynyk | |
| sptfcehcgt llwglarqgl kcdacgmnvh hrcqtkvanl acginqklmae | 301 |
| lamiestqq | |
| arclrdteqi fregpveigl pcsiknearp pclptpgkre | 361 |
| dpqgiswespl evdkmchlp | |
| epelnkerps lqiklkiedf ilhkmlgkgs fgkvflaefk ktnqffaika | 421 |
| lkkdvvlmdd | |
| dvectmvekr vlslawehpf lthmfctfqt kenlffvmey lnggdlmyhi | 481 |
| qschkfdlsr | |
| atfyaaeiil glqflhskgi vyrdlkldni lldkdghiki adfgmckenm | 541 |
| lgdaktntfc | |
| gtpdyiapei llgqkynhsv dwwsfgvlly emligqspfh gqdeeelfhs | 601 |
| irmdnpfypr | |
| wlekeakdll vklfvrepek rlgvrgdirq hplfreinwe elerkeidpp | 661 |
| frpkvkspfd | |
| csnfdkefln ekprlsfadr alinsmdqnm frnfsfmnpg merlis | |

Gorilla PKC-theta
MSPFLRIGLSNFDCGSCQSCQGEAVNPYCAVLVKEYVESENGQMYIQKKPIMYPPWDSIF
DAHINKGRVMQIIVKGKNVDLISETTVELYSLAERCRKNNGKTEIWLELKPQGRMLMNAR
YFLEMSDTKDMNEFETEGFFALHQRRGAIKQAKVHHVKCHEFTATFFPQPTFCSVCHEFV
WGLNKQGYQCRQCNAAIHKKCIDKVIAKCTGSAINSRETMFHKERFKIDMPHRFKVYNYK
SPTFCEHCGTLLWGLARQGLKCDACGMNVHHRCQTKVANLCGINQKLMAEALAMIESTQQ
ARCLRDTEQIFREGPVEIGLPCSIKNEARPPCLPTPGKREPQGISWESPLDEVDK
MCHLPEPELNIERPSLQIKLKIEDFILHKMLGKGSFGKVFLAEFKKTNQFFAIKALKKD
VVLMDDDVECTMVEKRVLSLAWEHPFLTHMFCTFQTKENLFFVMEYLNGGDLMYHIQSCHKF
DLSRATFYAAEIILGLQFLHSKGIVYRDLKLDNILLDKDGHIKIADFGMCKENMLGDAK
TNTFCGTPDYIAPEILLGQKYNHSVDWWSFGVLLYEMLIGQSPFHGQDEEELFHSIRMD
NPFYPRWLEKEAKDLLVKLFVREPEKRLGVRGDIRQHPLFREINWEELERKEIDPPFRP
KVKSPFDCSNFDKEFLNEKPRLSFADRALINSMDQNMFRNFSFMNPGMERLIS
Chimpanzee PKC-theta
MSPFLRIGLSNFDCGSCQSCQGEAVNPYCAVLVKEYVESENGQMYIQKKPTMYPPWDSTF
DAHINKGRVMQIIVKGKNVDLISETTVELYSLAERCRKNNGKTEIWLELKPQGRMLMNAR
YFLEMSDTKDMNEFETEGFFALHQRRGAIKQAKVHHVKCHEFTATFFPQPTFCSVCHEFV
WGLNKQGYQCRQCNAAIHKKCIDKVIAKCTGSAINSRETMFHKERFKIDMPHRFKVYNYK
SPTFCEHCGTLLWGLARQGLKCDACGMNVHHRCQTKVANLCGINQKLMAEALAMIESTQQ
ARCLRDTEQIFREGPVEIGLPCSIKNEARPPCLPTLGKREPQGISWESPLDE
VDKMCHLPEPELNIERPSLQIKLKIEDFILHKMLGKGSFGKVFLAEFKKTNQFFAIKAL
KKDVVLMDDDVECTMVEKRVLSLAWEHPFLTHMFCIFQTKENLFFVMEYLNGGDLMYHIQ
SCHKFDLSRATFYAAEIILGLQFLHSKGIVYRDLKLDNILLDKDGHIKIADFGMCKENMLG
DAKTNTFCGTPDYIAPEILLGQKYNHSVDWWSFGVLLYEMLIGQSPFHGQDEEELFHSIR
MDNPFYPRWLEKEAKDLLVKLFVREPEKRLGVRGDIRQHPLFREINWEELERKEIDPPFR
KPKVSPFDCSNFDKEFLNEKPRLSFADRALINSMDQNMFRNFSFMNPGMERLIS
Orangutan PKC-theta
MSPFLRIGLSNFDCGSCQSCQGEAVNPYCAVLVKEYVESENGQMYIQKKPTMYPPWDSTF
DAHINKGRVMQIIVKGKNVDLISETTVELYSLAERCRKNNGKTEIWLELKPQGRMLMNAR
YFLEMSDTKDMSEFEMEGFFALHQRRGAIKQAKVHHVKCHEFTATFFPQPTFCSVCHEFV
WGLNKQGYQCRQCNAAIHKKCIDKVIAKCTGSAINSRETMFHKERFKIDMPHRFKVYNYK
SPTFCEHCGTLLWGLARQGLKCDACGMNVHHRCQTKVANLCGINQKLMAEALAMIESTQQ
ARCLRDTEQIFREGPVEIGLPCSIKNEARPLCLPTPGKREPQGISWESPLDEVDK
MCHLPEPELTIERPSLQMKLKIEDFILHKMLGKGSFGKVFLAEFKKTNQFFAIKTLKKD
VVLMDDDVECTMVEKRVLSLAWEHPFLTHMFCTFQTKENLFFVMEYLNGGDLMYHIQSCH
KFDLSRATFYAAEIILGLQFLHSKGIVYRDLKLDNILLDKDGHIKIADFGMCKENMLGDAK
TNTFCGTPDYIAPEILLGQKYNHSVDWWSFGVLLYEMLIGQSPFHGQDEEELFHSIRMDNP
FYPRWLEKEAKDLLVKLFVREPEKRLGVRGDIRQHPLFREINWEELERKEIDPPFRPKVKS
PYDCSNFDKEFLNEKPRLSFADRALINSMDQNMFRNFSFMNPGMERLIS
Macaque PKC-theta
MIKHWLSRRGTPKTVPFIAPKQHLSCVVFQGATMSPFLRIGLSNFDCGSCQSCQGEAVNP
YCAVLVKEYVESENGQMYIQKKPTMYPPWDSTFDAHINKGRVMQIIVKGKNVDLISETTV
ELYSLAERCRKNNGKTEIWLELKPQGRMLMNARYFLEMSDTKDMSEFETEGFFALHQRRG
AIKQAKVHHVKCHEFTATFFPQPTFCSVCHEFVWGLNKQGYQCRQCNAAIHKKCIDKVIA
KCTGSAINSRETMFHKERFKIDMPHRFKVYNYKSPTFCEHCGTLLWGLARQGLKCDACGM
NVHHRCQTKVANLCGINQKLMAEALAMIESTQQARCLRDTEQIFREGPVEIGLPCSTKNE
ARPPCLPTPGKREPQGISWESPLDEVDKMCHLPEPELNKERPSLQMKLKIEDFILHKMLG
KGSFGKVFLAEFKKTNQFFAIKALKKDVVLMDDDVECTMVEKRVLSLAWEHPFLTHMFCT
FQTKENLFFVMEYLNGGDLMYHIQSCHKFDLSRATFYAAEIILGLQFLHSKGIVYRDLKL
DNILLDKDGHIKIADFGMCKENMLGDAKTNIFCGTPDYIAPEILLGQRYNHSVDWWSFGV
LLYEMLIGQSPFHGQDEEELFHSIRMDNPFYPRWLEKEAKDLLVKLFVREPEKRLGVRGD
IRQHPLFREINWEELERKEIDPPFRPKVKSPYDCSNFDKEFLNEKPRLSFADRALINSMD
QNMFRNFSFMNPGMERLIS

Non-limiting exemplary full length human Lkc polypeptide sequence showing the SH2 (bold) and SH3 (underlined) domains, as disclosed herein, is as follows (SEQ ID NO:6):

| | |
|---|---|
| 1 | mgcgcsshpe ddwmenidvc enchypivpl dgkgtllirn gsevrdplvt yegsnppasp |
| 61 | lqdnlvialh syepshdgdl gfekgeqlri leqsgewwka gslttggegf ipfnfvakan |
| 121 | slepepwffk nlsrkdaerq llapgnthgs fliresesta gsfslsvrdf dqnqgevvkh |
| 181 | ykirnldngg fyispritfp glhelvrhyt nasdglctrl srpcqtqkpq kpwwedewev |
| 241 | pretlklver lgagqfgevw mgyynghtkv avkslkqgsm spdaflaean lmkqlqhqrl |
| 301 | vrlyavvtqe piyiiteyme ngslvdflkt psgikltink lldmaaqiae gmafieerny |
| 361 | ihrdlraani lvsdtlscki adfglarlie dneytarega kfpikwtape ainygtftik |
| 421 | sdvwsfgill teivthgrip ypgmtnpevi qnlergyrmv rpdncpeely qlmrlcwker |
| 481 | pedrptfdyl rsvledffta tegqyqpqp |

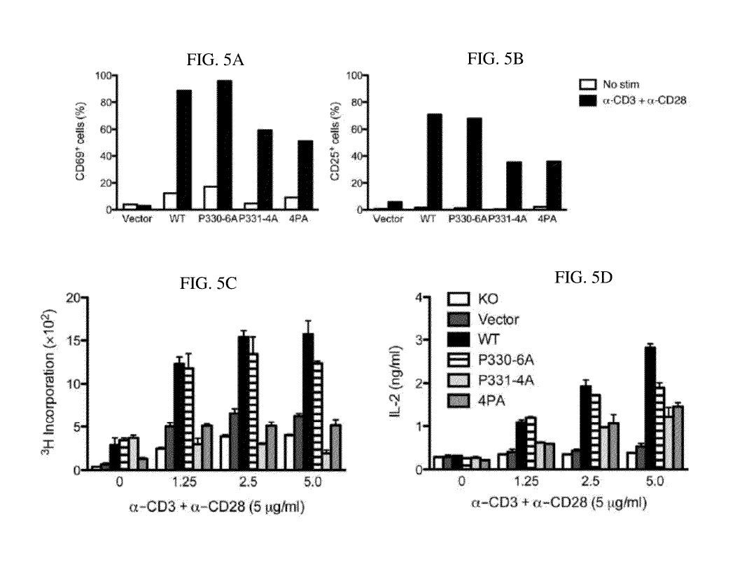

Lck SH2 domain is believed to interact with the phosphoprylated PY*AP motif of CD28. Lck SH3 domain is believed to interact with the proline-rich motif in the V3 domain of PKCθ (see, e.g., FIG. 5).

Non-limiting exemplary full length CD28 sequences, Isoforms 1, 2 and 3, are as follows:

Isoform 1
(220 aa, SEQ ID NO: 7):
MLRLLLALNLFPSIQVTGNKILVKQSPMLVAYDNAVNLSCKYSYNLFSREFRASLHKGLDS
AVEVCVVYGNYSQQLQVYSKTGFNCDGKLGNESVTFYLQNLYVNQTDIYFCKIEVMYPPP
YLDNEKSNGTIIHVKGKHLCPSPLFPGPSKPFWVLVVVGGVLACYSLLVTVAFIIFWVRSK
RSRLLHSDYMNMTPRRPGPTRKHYQPYAPPRDFAAYRS
Isoform 2
(123 aa, SEQ ID NO: 8):
MLRLLLALNLFPSIQVTGNKILVKQSPMLVAYDNAVNLSWKHLCPSPLFPGPSKPFWVLV
VVGGVLACYSLLVTVAFIIFWVRSKRSRLLHSDYMNMTPRRPGPTRKHYQPYAPPRDFAA
YRS
Isoform 3
(101 aa, SEQ ID NO: 9):
MLRLLLALNLFPSIQVTGKHLCPSPLFPGPSKPFWVLVVVGGVLACYSLLVTVAFIIFWVR
SKRSRLLHSDYMNMTPRRPGPTRKHYQPYAPPRDFAAYRS

As used herein, a “polypeptide” or “peptide” refers to two, or more, amino acids linked by an amide or equivalent bond. A polypeptide can also be referred to herein, inter alia, as a protein, or an amino acid sequence, or simply a sequence. Polypeptides of the invention include L- and D-isomers, and combinations of L- and D-isomers. Polypeptides can form intra or intermolecular disulfide bonds. Polypeptides can also form higher order structures, such as multimers or oligomers, with the same or different polypeptide, or other molecules. The polypeptides can include modifications typically associated with post-translational processing of proteins, for example, cyclization (e.g., disulfide bond), phosphorylation, glycosylation, carboxylation, ubiquitination, myristylation, acetylation (N-terminal), amidation (C-terminal), or lipidation. Polypeptides described herein further include compounds having amino acid structural and functional analogues, for example, peptidomimetics having synthetic or non-natural amino acids or amino acid analogues, so long as the mimetic has one or more functions or activities of a native polypeptide set forth herein. Non-natural and non-amide chemical bonds, and other coupling means can also be included, for example, glutaraldehyde, N-hydroxysuccinimide esters, bifunctional maleimides, or N,N′-dicyclohexylcarbodiimide (DCC). Non-amide bonds can include, for example, ketomethylene aminomethylene, olefin, ether, thioether and the like (see, e.g., Spatola (1983) in Chemistry and Biochemistry of Amino Acids, Peptides and Proteins, Vol. 7, pp 267-357, “Peptide and Backbone Modifications,” Marcel Decker, NY).

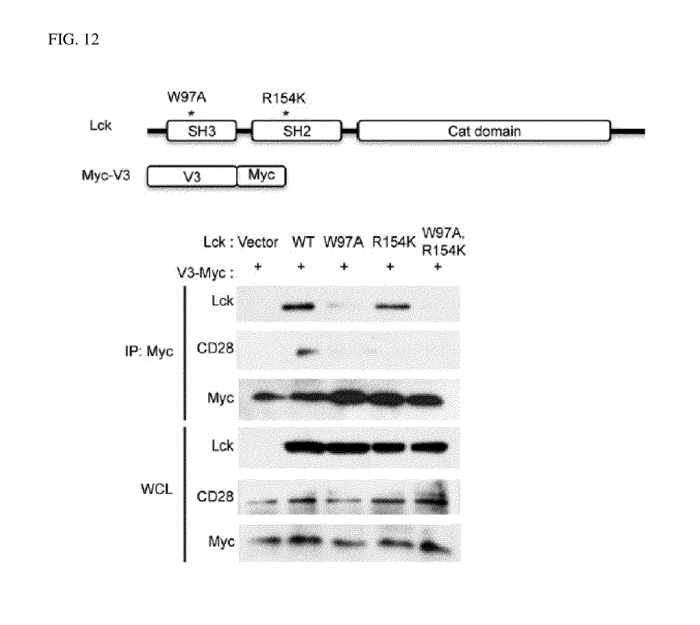

As set forth herein and in particular aspects, a PKCθ, CD28 or Lck sequence can inhibit, reduce or decrease binding between PKCθ to CD28. The term “bind,” or “binding,” when used in reference to an interaction between PKCθ and CD28 means that there is a physical interaction at the molecular level or functional interaction between PKCθ and CD28. As Lck is an intermediate in binding between PKCθ and CD28 (FIG. 12), the interaction can also include interaction of PKCθ and CD28 and Lck. A functional interaction need not require physical binding. Thus, an inhibitor of binding between PKCθ and CD28 partially or completely inhibits, decreases or reduces a physical interaction or a functional interaction between PKCθ and CD28. Binding inhibition can be due to steric hinderance, occupation or blocking of the site of physical or functional interaction or alteration of a modification or another factor that participates in binding between PKCθ and CD28. Accordingly, inhibitors of binding between PKCθ and CD28 can act directly or indirectly upon PKCθ and/or CD28 and/or Lck. For example, a peptide comprising the CD28 binding region of PKCθ can be an inhibitor that binds to the CD28, or the CD28 or PKCθ binding region of Lck, can be an inhibitor that binds to the CD28 or PKCθ, thereby inhibiting binding between PKCθ and CD28.

As disclosed herein, a PXXP motif in PKCθ appears to participate in and/or mediate binding to CD28. As also disclosed herein, a PYAP motif present in CD28 appears to participate in and/or mediate binding to PKCθ. As further disclosed herein, a SH2 and/or SH3 domain present in Lck appears to participate in and/or mediate binding between PKCθ and CD28. Accordingly inhibitors can inhibit, decrease or reduce binding between PKCθ and CD28 by interference of a physical or functional interaction of either of these two motifs, for example.

In accordance with the invention and in particular embodiments, a PKCθ sequence includes or consists of a PXXP motif. In further particular embodiments, a PKCθ amino acid sequence includes or consists of a ARPPCLPTP, ETRPPCVPTPGK, ARLPCVPAP, ARLPCVPAS, AKLPHAPAP, AKPPYVPGP, TRLPYLPTP, any other sequence motif set forth in Table 1 (Example 4), a subsequence thereof, or a sequence variant of ARPPCLPTP, ETRPPCVPTPGK, ARLPCVPAP, ARLPCVPAS, AKLPHAPAP, AKPPYVPGP, TRLPYLPTP or sequence motif set forth in Table 1 (e.g., a substitution of a first or last proline residue with another amino acid, such as alanine), or a subsequence thereof. In accordance with the invention and in additional particular embodiments, a CD28 sequence includes or consists of a PYAP motif, which corresponds to amino acids 206-209 of CD28. Also in accordance with the invention and in additional particular embodiments, a Lck sequence includes or consists of an SH2 and/or SH3 domain (or a subsequence or fragment of an SH2 and/or SH3 domain), as set forth herein.

Accordingly, PKCθ, CD28 and Lck sequences, subsequences and fragments, and substitutions, variants and polymorphisms of the invention, as well as methods and uses of the invention including PKCθ, CD28 and Lck sequences, subsequences and fragments, amino acid substitutions, variants and polymorphisms include but are but not limited to PXXP motifs including subsequences, substitutions, variants and polymorphisms thereof, such as the non-limiting motifs set forth in Table 1 or a substitution of a first or last proline residue with another amino acid, and PYAP motifs containing subsequences, substitutions, variants and polymorphisms thereof. Such forms can be conveniently referred to as variant or modified forms of PKCθ, CD28 and Lck.

As set forth herein, modified and variant forms also include, for example, in addition to subsequences and fragments, deletions, substitutions, additions, and insertions of the amino acid sequences set forth herein, such as PKCθ and/or CD28 and/or Lck Exemplary sequence deletions, substitutions, additions, and insertions include a full length sequence or a subsequence with one or more amino acids deleted, substituted, added or inserted.

Subsequences, and fragments, variants and modified forms, and polymorphisms can be considered functional as long as they retain at least a partial function or activity of a reference molecule. For example, a functional PKCθ subsequence, variant, modified form, or polymorphism would retain at least a partial function or activity of full-length PKCθ; a functional CD28 subsequence, variant or modified form, or polymorphism would retain at least a partial function or activity of full-length CD28; and a functional Lck subsequence, variant or modified form, or polymorphism would retain at least a partial function or activity of full-length lck (e.g., binding to CD28 or PKCθ).

A “functional sequence” or “functional variant,” or “functional polymorphism,” as used herein refers to a sequence, subsequence, variant or modified form, or polymorphism that possesses at least one partial function or activity characteristic of a native wild type or full length counterpart polypeptide. For example, PKCθ, CD28 or Lck polypeptide subsequence, variant or modified form, or polymorphism, as disclosed herein, can function to modulate (e.g., inhibit, reduce or decrease) binding between PKCθ and CD28. The invention therefore includes PKCθ, CD28 and Lck sequences, subsequences, and fragments, variants and modified forms, and polymorphismsthat typically retain, at least a part of, one or more functions or activities of a corresponding reference or an unmodified native wild type or full length counterpart PKCθ, CD28 or Lck sequence. Compositions, methods and uses of the invention therefore include PKCθ, CD28 and Lck polypeptide sequences, subsequences, variants and modified forms, and polymorphisms, having one or more functions or activities of wild type native PKCθ, CD28 and Lck.

As disclosed herein, inhibition of binding between PKCθ and CD28 polypeptide can lead to various effects on one or more PKCθ and/or CD28 functions or activities. Particular non-limiting examples include modulating, such as decreasing, reducing, inhibiting, suppressing, limiting or controlling an undesirable or aberrant immune response, immune disorder, inflammatory response, or inflammation; modulating, such as decreasing, reducing, inhibiting, suppressing, limiting or controlling an autoimmune response, disorder or disease; and modulating, such as increasing, inducing, stimulating, or promoting regulatory T cell (Tregs) differentiation or function. Accordingly, functional sequences therefore include subsequences, variants and modified forms, and polymorphisms, such as PKCθ, CD28 and Lck sequences that, may have one or more of functions or biological activities described herein or known to one of skill in the art (e.g., ability to modulate binding between PKCθ and CD28; modulation of undesirable or aberrant immune responses, immune disorders, inflammatory responses, or inflammation; modulation of autoimmune responses, disorders or diseases; modulation of regulatory T cell (Tregs) differentiation or function, etc.)

PKCθ, CD28 and Lck sequences, subsequences, variants and modified forms, and polymorphisms may have an activity or function greater or less than 2-5, 5-10, 10-100, 100-1000 or 1000-10.000-fold activity or function than a comparison PKCθ, CD28 or Lck sequence. For example, a PKCθ, CD28 or Lck sequence, subsequence or a modified or variant form could have a function or activity greater or less than 2-5, 5-10, 10-100, 100-1000 or 1000-10.000-fold function or activity of a reference PKCθ, CD28 or Lck to modulate (e.g., decrease, reduce, or inhibit) binding between PKCθ and CD28, or to modulate an undesirable or aberrant immune response, immune disorder, inflammatory response, or inflammation; modulate an autoimmune response, disorder or disease; or modulate regulatory T cell (Tregs) differentiation or function.

In particular embodiments, a functional sequence shares at least 50% identity with a reference sequence, for example, a PKCθ or CD28 or Lck polypeptide sequence that is capable of modulating (e.g., inhibiting, reducing or decreasing) binding of PKCθ to CD28, or modulating an activity, function or expression of PKCθ and/or CD28. In other embodiments, the sequences have at least 60%, 70%, 75% or more identity (e.g., 80%, 85% 90%, 95%, 96%, 97%, 98%, 99% or more identity) to a reference sequence.

The term “identity” and grammatical variations thereof, mean that two or more referenced entities are the same. Thus, where two polypeptide (e.g., PKCθ, CD28 or Lck) sequences are identical, they have the same amino acid sequence, at least within the referenced region or portion. Where two nucleic acid sequences are identical, they have the same polynucleotide sequence, at least within the referenced region or portion. The identity can be over a defined area (region or domain) of the sequence. An “area of identity” refers to a portion of two or more referenced entities that are the same. Thus, where two protein or nucleic acid sequences are identical over one or more sequence regions they share identity within that region.

The percent identity can extend over the entire sequence length of the polypeptide (e.g., PKCθ, CD28 or Lck). In particular aspects, the length of the sequence sharing the percent identity is 5 or more contiguous amino acids, e.g., 5, 6, 7, 8, 9, 10, 11, 12, 13, 14, 15, 16, 17, 18, 19, 20, 21, 22, 23, 24, 25, etc. contiguous amino acids. In additional particular aspects, the length of the sequence sharing the percent identity is 25 or more contiguous amino acids, e.g., 26, 27, 28, 29, 30, 31, 32, 33, 34, 35, etc. contiguous amino acids. In further particular aspects, the length of the sequence sharing the percent identity is 35 or more contiguous amino acids, e.g., 35, 36, 37, 38, 39, 40, 41, 42, 43, 44, 45, 45, 47, 48, 49, 50, etc., contiguous amino acids. In yet additional particular aspects, the length of the sequence sharing the percent identity is 50 or more contiguous amino acids, e.g., 50-55, 55-60, 60-65, 65-70, 70-75, 75-80, 80-85, 85-90, 90-95, 95-100, 100-110, etc. contiguous amino acids.

The terms “homologous” or “homology” mean that two or more referenced entities share at least partial identity over a given region or portion. “Areas, regions or domains” of homology or identity mean that a portion of two or more referenced entities share homology or are the same. Thus, where two sequences are identical over one or more sequence regions they share identity in these regions. “Substantial homology” means that a molecule is structurally or functionally conserved such that it has or is predicted to have at least partial structure or function of one or more of the structures or functions (e.g., a biological function or activity) of the reference molecule, or relevant/corresponding region or portion of the reference molecule to which it shares homology. A PKCθ, CD28 or Lck sequence, or a subsequence, variant or modified form, or polymorphism with substantial homology has or is predicted to have at least partial activity or function as the reference sequence.

The extent of identity (homology) between two sequences can be ascertained using a computer program and mathematical algorithm known in the art. Such algorithms that calculate percent sequence identity (homology) generally account for sequence gaps and mismatches over the comparison region or area. For example, a BLAST (e.g., BLAST 2.0) search algorithm (see, e.g., Altschul et al., J. Mol. Biol. 215:403 (1990), publicly available through NCBI) has exemplary search parameters as follows: Mismatch-2; gap open 5; gap extension 2. For polypeptide sequence comparisons, a BLASTP algorithm is typically used in combination with a scoring matrix, such as PAM100, PAM 250, BLOSUM 62 or BLOSUM 50. FASTA (e.g., FASTA2 and FASTA3) and SSEARCH sequence comparison programs are also used to quantitate extent of identity (Pearson et al., Proc. Natl. Acad. Sci. USA 85:2444 (1988); Pearson, Methods Mol. Biol. 132:185 (2000); and Smith et al., J. Mol. Biol. 147:195 (1981)). Programs for quantitating protein structural similarity using Delaunay-based topological mapping have also been developed (Bostick et al., Biochem Biophys Res Commun. 304:320 (2003)).

Modified and variant polypeptides include, for example, non-conservative and conservative substitutions of PKCθ, CD28 and Lck sequences. In particular embodiments, a modified protein has one or a few (e.g., 1-5%, 5-10%, 10-20%) of the residues of total protein length, or 1-2, 2-3, 3-4, 5-10, 10-20, 20-50 residues substituted, with conservative or non-conservative substitutions or conservative and non-conservative amino acid substitutions. A “conservative substitution” denotes the replacement of an amino acid residue by another, chemically or biologically similar residue. Biologically similar means that the substitution does not destroy a biological activity or function. Structurally similar means that the amino acids have side chains with similar length, such as alanine, glycine and serine, or a similar size. Chemical similarity means that the residues have the same charge or are both hydrophilic or hydrophobic.

Particular examples of conservative substitutions include the substitution of a hydrophobic residue such as isoleucine, valine, leucine or methionine for another, the substitution of a polar residue for another, such as the substitution of arginine for lysine, glutamic for aspartic acids, or glutamine for asparagine, and the like. A “conservative substitution” also includes the use of a substituted amino acid in place of an unsubstituted parent amino acid.

Modified and variant proteins also include one or more D-amino acids substituted for L-amino acids (and mixtures thereof), structural and functional analogues, for example, peptidomimetics having synthetic or non-natural amino acids or amino acid analogues and derivatized forms. Modified and variant proteins further include “chemical derivatives,” in which one or more amino acids has a side chain chemically altered or derivatized. Such derivatized polypeptides include, for example, amino acids in which free amino groups form amine hydrochlorides, p-toluene sulfonyl groups, carobenzoxy groups; the free carboxy groups form salts, methyl and ethyl esters; free hydroxl groups that form O-acyl or O-alkyl derivatives as well as naturally occurring amino acid derivatives, for example, 4-hydroxyproline, for proline, 5-hydroxylysine for lysine, homoserine for serine, ornithine for lysine etc. Also included are amino acid derivatives that can alter covalent bonding, for example, the disulfide linkage that forms between two cysteine residues that produces a cyclized polypeptide.

Additions and insertions include, for example, heterologous domains. An addition (e.g., heterologous domain) can be a covalent or non-covalent attachment of any type of molecule to a composition, such as a protein (e.g. PKCθ, CD28 or Lck) or other chemical entity (e.g. organic or inorganic compound). Typically additions and insertions (e.g., a heterologous domain) confer a complementary or a distinct function or activity.

Additions and insertions include chimeric and fusion sequences, which is a protein sequence having one or more molecules not normally present in a reference native wild type sequence covalently attached to the sequence. The terms “fusion” or “chimeric” and grammatical variations thereof, when used in reference to a molecule, such as PKCθ, CD28 or Lck, means that a portions or part of the molecule contains a different entity distinct (heterologous) from the molecule (e.g., PKCθ, CD28 or Lck) as they do not typically exist together in nature. That is, for example, one portion of the fusion or chimera, such as PKCθ, includes or consists of a portion that does not exist together in nature, and is structurally distinct. A particular example is a molecule, such as amino acid residues or a polypeptide sequence of another protein (e.g., cell penetrating moiety or protein such as HIV tat) attached to PKCθ, CD28 or a Lck subsequence to produce a chimera, or a chimeric polypeptide, to impart a distinct function (e.g., increased cell penetration).

In particular embodiments, additions and insertions include a cell-penetrating moiety (CPM), or a cell-penetrating peptide (CPP). As used herein, a “cell-penetrating moiety (CPM)” is a molecule that penetrates or passes through cell membranes, typically without a need for binding to a cell membrane receptor. A cell penetrating peptide (CPP) can penetrate membranes, and is typically a peptide sequence of less that 25-50 (more typically, 30) amino acid residues in length. In particular non-limiting aspects, a CPM or CPP includes HIV Tat, Drosophila antennapedia (RQIKIWFQNRRMKWKK), polyarginine (RRRRRRRRR), polylysine (KKKKKKKKK), PTD-5 (RRQRRTSKLMKR), or a Transportan (GWTLNSAGYLLGKINLKALAALAKKIL), or KALA (WEAKLAKALAKALAKHLAKALAKALKACEA) sequence.

Additions and insertions further include labels and tags, which can be used to provide detection or that is useful for isolating the tagged entity (e.g., PKCθ, CD28 or lck sequence). A detectable label can be attached (e.g., linked or conjugated), for example, to a PKCθ or a CD28 sequence, or be within or comprise one or more atoms that comprise the molecule.

Non-limiting exemplary detectable labels include a radioactive material, such as a radioisotope, a metal or a metal oxide. Radioisotopes include radionuclides emitting alpha, beta or gamma radiation, such as one or more of: ³H, ¹⁰B, ¹⁸F, ¹¹C, ¹⁴C, ¹³N, ¹⁸O, ¹⁵O, ³²P, P³³, ³⁵S, ³⁵Cl, ⁴⁵Ti, ⁴⁶Sc, ⁴⁷Sc, ⁵¹Cr, ⁵²Fe, ⁵⁹Fe, ^{.57}Co, ⁶⁰Cu, ⁶¹Cu, ⁶²Cu, ⁶⁴Cu, ⁶⁷Cu, ⁶⁷Ga, ⁶⁸Ga, ⁷²As ⁷⁶Br, ⁷⁷Br, ^{81m}Kr, ⁸²Rb, ⁸⁵Sr, ⁸⁹Sr, ⁸⁶Y, ⁹⁰Y, ⁹⁵Nb, ^{94m}Tc, ^{99m}Tc, ⁹⁷Ru, ¹⁰³Ru, ¹⁵⁰Rh, ¹⁰⁹Cd, ¹¹¹In, ¹¹³Sn, ^{113m}In, ¹¹⁴In, I¹²⁵, I¹³¹, ¹⁴⁰La, ¹⁴¹Ce, ¹⁴⁹Pm, ¹⁵³Gd, ¹⁵⁷Gd, ¹⁵³Sm, ¹⁶¹Tb, ¹⁶⁶Dy, ¹⁶⁶Ho, ¹⁶⁹Er, ¹⁶⁹Y, ¹⁷⁵Yb, ¹⁷⁷Lu, ¹⁸⁶Re, ¹⁸⁸Re, ²⁰¹Tl, ²⁰³Pb, ²¹¹At, ²¹²Bi or ²²⁵Ac. Additional non-limiting exemplary detectable labels include a metal or a metal oxide, such as gold, silver, copper, boron, manganese, gadolinium, iron, chromium, barium, europium, erbium, praseodynium, indium, or technetium.

Further non-limiting exemplary detectable labels include contrast agents (e.g., gadolinium; manganese; barium sulfate; an iodinated or noniodinated agent; an ionic agent or nonionic agent); magnetic and paramagnetic agents (e.g., iron-oxide chelate); nanoparticles; an enzyme (horseradish peroxidase, alkaline phosphatase, β-galactosidase, or acetylcholinesterase); a prosthetic group (e.g., streptavidin/biotin and avidin/biotin); a fluorescent material (e.g., umbelliferone, fluorescein, fluorescein isothiocyanate, rhodamine, dichlorotriazinylamine fluorescein, dansyl chloride or phycoerythrin); a luminescent material (e.g., luminol); or a bioluminescent material (e.g., luciferase, luciferin, aequorin).

Still further non-limiting tags and/or detectable labels include enzymes (horseradish peroxidase, urease, catalase, alkaline phosphatase, beta-galactosidase, chloramphenicol transferase); enzyme substrates; ligands (e.g., biotin); receptors (avidin); GST-, T7-, His-, myc-, HA- and FLAG-tags; electron-dense reagents; energy transfer molecules; paramagnetic labels; fluorophores (fluorescein, fluorscamine, rhodamine, phycoerthrin, phycocyanin, allophycocyanin); chromophores; chemi-luminescent (imidazole, luciferase, acridinium, oxalate); and bio-luminescent agents.

As set forth herein, a detectable label or tag can be linked or conjugated (e.g., covalently) to the molecule (e.g., PKCθ, a CD28 or Lck sequence). In various embodiments a detectable label, such as a radionuclide or metal or metal oxide can be bound or conjugated to the agent, either directly or indirectly. A linker or an intermediary functional group can be used to link the molecule to a detectable label or tag. Linkers include amino acid or peptidomimetic sequences inserted between the molecule and a label or tag so that the two entities maintain, at least in part, a distinct function or activity. Linkers may have one or more properties that include a flexible conformation, an inability to form an ordered secondary structure or a hydrophobic or charged character which could promote or interact with either domain. Amino acids typically found in flexible protein regions include Gly, Asn and Ser. The length of the linker sequence may vary without significantly affecting a function or activity.

Linkers further include chemical moieties, conjugating agents, and intermediary functional groups. Examples include moieties that react with free or semi-free amines, oxygen, sulfur, hydroxy or carboxy groups. Such functional groups therefore include mono and bifunctional crosslinkers, such as sulfo-succinimidyl derivatives (sulfo-SMCC, sulfo-SMPB), in particular, disuccinimidyl suberate (DSS), BS3 (Sulfo-DSS), disuccinimidyl glutarate (DSG) and disuccinimidyl tartrate (DST). Non-limiting examples include diethylenetriaminepentaacetic acid (DTPA) and ethylene diaminetetracetic acid.

Modifications can be produced using methods known in the art (e.g., PCR based site-directed, deletion and insertion mutagenesis, chemical modification and mutagenesis, cross-linking, etc.), or may be spontaneous or naturally occurring (e.g. random mutagenesis). For example, naturally occurring allelic variants can occur by alternative RNA splicing, polymorphisms, or spontaneous mutations of a nucleic acid encoding PKCθ, CD28 or Lck sequence. Further, deletion of one or more amino acids can also result in a modification of the structure of the resultant polypeptide without significantly altering a biological function or activity. Deletion of amino acids can lead to a smaller active molecule. For example, as set forth herein, removal of PKCθ amino acids does not destroy the ability of such a modified PKCθ to inhibit binding between PKCθ and CD28.

The term “isolated,” when used as a modifier of a composition (e.g., PKCθ, CD28 or Lck sequences, subsequences, variant and modified forms, etc.), means that the compositions are made by the hand of man or are separated, completely or at least in part, from their naturally occurring in vivo environment. Generally, isolated compositions are substantially free of one or more materials with which they normally associate with in nature, for example, one or more protein, nucleic acid, lipid, carbohydrate, cell membrane. The term “isolated” does not exclude alternative physical forms, such as fusions/chimeras, multimers/oligomers, modifications (e.g., phosphorylation, glycosylation, lipidation) or derivatized forms, or recombinant or other forms expressed in vitro, in host cells, or in an animal and produced by the hand of man.

An “isolated” composition (e.g., a PKCθ, CD28 or Lck sequence) can also be “substantially pure” or “purified” when free of most or all of the materials with which it typically associates with in nature. Thus, an isolated sequence that also is substantially pure or purified does not include polypeptides or polynucleotides present among millions of other sequences, such as antibodies of an antibody library or nucleic acids in a genomic or cDNA library, for example. Typically, purity can be at least about 50%, 60% or more by mass. The purity can also be about 70% or 80% or more, and can be greater, for example, 90% or more. Purity can be determined by any appropriate method, including, for example, UV spectroscopy, chromatography (e.g., HPLC, gas phase), gel electrophoresis and sequence analysis (nucleic acid and peptide), and is typically relative to the amount of impurities, which typically does not include inert substances, such as water.

A “substantially pure” or “purified” composition can be combined with one or more other molecules. Thus, “substantially pure” or “purified” does not exclude combinations of compositions, such as combinations of PKCθ, CD28 or Lck sequences, subsequences, variants and modified forms, and other molecular entities such as agents, drugs or therapies.

As used herein, the term “recombinant,” when used as a modifier of sequences such as polypeptides and polynucleotides, means that the compositions have been manipulated (i.e., engineered) in a fashion that generally does not occur in nature (e.g., in vitro). A particular example of a recombinant polypeptide would be where a PKCθ, CD28 or Lck polypeptide is expressed by a cell transfected with a polynucleotide encoding the PKCθ, CD28 or Lck sequence. A particular example of a recombinant polynucleotide would be where a nucleic acid (e.g., genomic or cDNA) encoding PKCθ or CD28 cloned into a plasmid, with or without 5′, 3′ or intron regions that the gene is normally contiguous with in the genome of the organism. Another example of a recombinant polynucleotide or polypeptide is a hybrid or fusion sequence, such as a chimeric PKCθ, CD28 or Lck sequence comprising a second sequence, such as a heterologous functional domain.

The invention also provides polynucleotides encoding PKCθ, CD28 or Lck sequences that modulate binding between PKCθ and CD28. In one embodiment, a polynucleotide sequence has about 65% or more identity (e.g., 70%, 75%, 80%, 85%, 90%, 95%, 96%, 97%, 98%, 99% or more) to a sequence encoding a PKCθ, CD28 or Lck subsequence that modulates binding between PKCθ and CD28. In particular embodiments, a nucleic acid encodes amino acids of a PKCθ PXXP motif or a PYAP motif of CD28. Such polynucleotides can therefore encode any subsequence of PKCθ, CD28 or Lck sequence that includes or consists of a region that binds to PKCθ or CD28, or that modulates binding between PKCθ and CD28.

As used herein, the terms “polynucleotide” and “nucleic acid” are used interchangeably to refer to all forms of nucleic acid, oligonucleotides, primers, and probes, including deoxyribonucleic acid (DNA) and ribonucleic acid (RNA). Polynucleotides include genomic DNA, cDNA and antisense DNA, and spliced or unspliced mRNA, rRNA tRNA and antisense RNA (e.g., RNAi). Polynucleotides include naturally occurring, synthetic, and intentionally altered or modified polynucleotides as well as analogues and derivatives. Alterations can result in increased stability due to resistance to nuclease digestion, for example. Polynucleotides can be double, single or triplex, linear or circular, and can be of any length.

Polynucleotides include sequences that are degenerate as a result of the genetic code. There are 20 natural amino acids, most of which are specified by more than one codon. Degenerate sequences may not selectively hybridize to other invention nucleic acids; however, they are nonetheless included as they encode PKCθ, CD28 or Lck sequences, subsequences, variants and modified forms, and polymorphisms thereof. Thus, in another embodiment, degenerate nucleotide sequences that encode PKCθ, CD28 or Lck sequences, subsequences, modified and variants forms, and polymorphisms, as set forth herein, are provided.

Polynucleotide sequences include sequences having 15-20, 20-30, 30-40, 50-50, or more contiguous nucleotides. In additional aspects, the polynucleotide sequence includes a sequence having 60 or more, 70 or more, 80 or more, 100 or more, 120 or more, 140 or more, 160 or more contiguous nucleotides, up to the full length coding sequence.

Polynucleotide sequences include complementary sequences (e.g., antisense to all or a part of PKCθ, CD28 or Lck). Antisense may be encoded by a nucleic acid and such a nucleic acid may be operatively linked to an expression control element for sustained or increased expression of the encoded antisense in cells or in vivo.

Polynucleotides can be obtained using various standard cloning and chemical synthesis techniques. Purity of polynucleotides can be determined through sequencing, gel electrophoresis and the like. For example, nucleic acids can be isolated using hybridization as set forth herein or computer-based database screening techniques known in the art. Such techniques include, but are not limited to: (1) hybridization of genomic DNA or cDNA libraries with probes to detect homologous nucleotide sequences; (2) antibody screening to detect polypeptides having shared structural features, for example, using an expression library; (3) polymerase chain reaction (PCR) on genomic DNA or cDNA using primers capable of annealing to a nucleic acid sequence of interest; (4) computer searches of sequence databases for related sequences; and (5) differential screening of a subtracted nucleic acid library.

PKCθ, CD28 and Lck polynucleotides can include an expression control element distinct from an endogenous PKCθ, CD28 or Lck gene (e.g., a non-native element), or exclude a control element from the native PKCθ, CD28 or Lck gene to control expression of an operatively linked nucleic acid. Such polynucleotides containing an expression control element controlling expression of a nucleic acid can be modified or altered as set forth herein, so long as the modified or altered polynucleotide has one or more functions or activities.

For expression in cells, polynucleotides, if desired, may be inserted into a vector. Accordingly, invention compositions and methods further include polynucleotide sequences inserted into a vector. The term “vector” refers to a plasmid, virus or other vehicle known in the art that can be manipulated by insertion or incorporation of a polynucleotide. Such vectors can be used for genetic manipulation (i.e., “cloning vectors”) or can be used to transcribe or translate the inserted polynucleotide (i.e., “expression vectors”). A vector generally contains at least an origin of replication for propagation in a cell and a promoter. Control elements, including expression control elements as set forth herein, present within a vector are included to facilitate proper transcription and translation (e.g., splicing signal for introns, maintenance of the correct reading frame of the gene to permit in-frame translation of mRNA and, stop codons etc.).

Invention compositions, methods and uses that include PKCθ and/or CD28 or Lck sequences can include any amount or dose of PKCθ, CD28 or Lck sequence, subsequence, variant or modified form, or polymorphism. In particular embodiments, PKCθ, CD28 or Lck is in a concentration range of about 10 μg/ml to 100 mg/ml, or in a range of about 100 μg/ml to 1,000 mg/ml, or at a concentration of about 1 mg/ml. In further particular embodiments, PKCθ, CD28 or Lck is in an amount of 10-1,000 milligrams, or an amount of 10-100 milligrams.

As disclosed herein, methods and uses of the invention include modulating (e.g., reducing, inhibiting, suppressing, or limiting) binding between PKCθ and CD28. Methods and uses of the invention can be performed in vivo, such as in a subject, in vitro, ex vivo, in a cell, in solution, in solid phase or in silica. In one embodiment, a method or use includes contacting an inhibitor of binding between CD28 and PKCθ thereby reducing, inhibiting, suppressing, or limiting binding between PKCθ and CD28.

As used herein, the term “modulate,” means an alteration or effect of the term modified. For example, the term modulate can be used in various contexts to refer to an alteration or effect of an activity, a function, or expression of a polypeptide, gene or signaling pathway, or a physiological condition or response of an organism. Methods and uses of the invention include modulating (e.g., decrease, reduce, inhibit, suppress, limit or control) one or more functions, activities or expression of PKCθ or CD28 in vitro, ex vivo or in vivo. Thus, where the term “modulate” is used to modify the term “PKCθ or CD28” this means that a PKCθ or CD28 activity, function, or expression is altered or affected (e.g., decreased, reduced, inhibited, suppressed, limited, controlled or prevented, etc.). Detecting an alteration or an effect on PKCθ or CD28 activity, function or expression can be determined as set forth herein using in vitro or in vivo assays, such as an animal model.

As disclosed herein, inhibition of binding between PKCθ and CD28 polypeptide can lead to various consequences, such as effects on a PKCθ and/or CD28 function or activity. Accordingly, PKCθ, CD28 and Lck sequences, subsequences, modified forms and variants, and polymorphisms as disclosed herein, including compositions including PKCθ and/or CD28 and/or Lck, are useful in various methods and uses such as treatment methods and uses, including, for example, treatment of numerous responses, disorders and diseases, both chronic and acute. In one embodiment, a method of treating a PKCθ mediated or dependent response, disorder, or disease, includes administering an inhibitor of binding between PKCθ and CD28 to a subject in an amount that treats the PKCθmediated or dependent response, disorder, or disease.

Responses, disorders and diseases include, without limitation, immune responses, disorders and diseases, inflammatory responses, disorders and diseases, and inflammation. Responses, disorders and diseases also include, without limitation, autoimmune responses, disorders and diseases. Responses additionally include regulatory T cell (Tregs) differentiation or function. Responses, disorders and diseases further include, without limitation, graft vs. host disease (GVHD), or host rejection of a cell, tissue or organ transplant (such as heart, liver, lung, bone marrow, etc.).

Accordingly, the invention provides methods and uses of modulating and treatment of all the foregoing responses, disorders and disease. In one embodiment, a method includes administering an inhibitor of binding between PKCθ and CD28 to a subject in an amount to decrease, reduce, inhibit, suppress, limit or control the undesirable or aberrant immune responses, disorders or diseases, inflammatory responses, disorders or diseases or inflammation in the subject. In another embodiment, a method includes administering an inhibitor of binding between PKCθ and CD28 to a subject in an amount to decrease, reduce, inhibit, suppress, limit or control an autoimmune response, disorder or disease in the subject. In an additional embodiment, a method includes contacting an inhibitor of binding between PKCθ and CD28 in an amount effective for increasing, inducing, stimulating, or promoting regulatory T cell differentiation or function. In a further embodiment, a method includes administering an inhibitor of binding between PKCθ and CD28 to a subject in an amount to decrease, reduce, inhibit, suppress, limit or control GVHD, or host rejection of a cell, tissue or organ transplant (such as heart, liver, lung, bone marrow, etc.).

Responses, disorders and diseases treatable in accordance with the invention include, but are not limited to, treatment of acute and chronic undesirable or aberrant immune responses, disorders or diseases, inflammatory responses, disorders or diseases or inflammation. Responses, disorders and diseases treatable in accordance with the invention also include, but are not limited to treatment of acute and chronic autoimmune responses, disorders and diseases. Such responses, disorders and diseases may be antibody or cell mediated, or a combination of antibody and cell mediated.

As used herein, an “undesirable immune response” or “aberrant immune response” refers to any immune response, activity or function that is greater or less than desired or physiologically normal response, activity or function including, acute or chronic responses, activities or functions. “Undesirable immune response” is generally characterized as an undesirable or aberrant increased or inappropriate response, activity or function of the immune system. However, an undesirable immune response, function or activity can be a normal response, function or activity. Thus, normal immune responses so long as they are undesirable, even if not considered aberrant, are included within the meaning of these terms. An undesirable immune response, function or activity can also be an abnormal response, function or activity. An abnormal (aberrant) immune response, function or activity deviates from normal.

One non-limiting example of an undesirable or aberrant immune response is where the immune response is hyper-responsive, such as in the case of an autoimmune disorder or disease. Another non-limiting example of an undesirable or aberrant immune response is where an immune response leads to acute or chronic inflammatory response or inflammation in any tissue or organ, such as an allergy, Crohn's disease, inflammatory bowel disease (IBD) or ulcerative colitis, or a transplant, as in GVHD (graft vs. host disease) or host rejection of a cell, tissue or organ transplant.

Undesirable or aberrant immune responses, inflammatory responses, or inflammation are characterized by many different physiological adverse symptoms or complications, which can be humoral, cell-mediated or a combination thereof. Responses, disorders and diseases that can be treated in accordance with the invention include, but are not limited to, those that either directly or indirectly lead to or cause cell or tissue/organ damage in a subject. At the whole body, regional or local level, an immune response, inflammatory response, or inflammation can be characterized by swelling, pain, headache, fever, nausea, skeletal joint stiffness or lack of mobility, rash, redness or other discoloration. At the cellular level, an immune response, inflammatory response, or inflammation can be characterized by one or more of T cell activation and/or differentiation, cell infiltration of the region, production of antibodies, production of cytokines, lymphokines, chemokines, interferons and interleukins, cell growth and maturation factors (e.g., proliferation and differentiation factors), cell accumulation or migration and cell, tissue or organ damage. Thus, methods and uses of the invention include treatment of and an ameliorative effect upon any such physiological symptoms or cellular or biological responses characteristic of immune responses, inflammatory response, or inflammation.

Autoimmune responses, disorders and diseases are generally characterized as an undesirable or aberrant response, activity or function of the immune system characterized by increased or undesirable humoral or cell-mediated immune responsiveness or memory, or decreased or insufficient tolerance to self-antigens. Autoimmune responses, disorders and diseases that may be treated in accordance with the invention include but are not limited to responses, disorders and diseases that cause cell or tissue/organ damage in the subject. The terms “immune disorder” and “immune disease” mean an immune function or activity, which is characterized by different physiological symptoms or abnormalities, depending upon the disorder or disease.

In particular embodiments, a method or use according to the invention decreases, reduces, inhibits, suppresses, limits or controls an undesirable or aberrant immune response, immune disorder, inflammatory response, or inflammation in a subject. In additional particular embodiments, a method or use decreases, reduces, inhibits, suppresses, limits or controls an autoimmune response, disorder or disease in a subject. In further particular embodiments, a method or use decreases, reduces, inhibits, suppresses, limits or controls an adverse symptom of the undesirable or aberrant immune response, immune disorder, inflammatory response, or inflammation, or an adverse symptom of the autoimmune response, disorder or disease.

In additional particular embodiments, methods and uses according to the invention can result in a reduction in occurrence, frequency, severity, progression, or duration of a symptom of the condition (e.g., undesirable or aberrant immune response, immune disorder, inflammatory response, or inflammation). For example, methods of the invention can protect against or decrease, reduce, inhibit, suppress, limit or control progression, severity, frequency, duration or probability of an adverse symptom of the undesirable or aberrant immune response, immune disorder, inflammatory response, or inflammation, or an autoimmune response, disorder or disease.

Examples of adverse symptoms of an undesirable or aberrant immune response, immune disorder, inflammatory response, or inflammation, or an adverse symptom of the autoimmune response, disorder or disease include swelling, pain, rash, discoloration, headache, fever, nausea, diarrhea, bloat, lethargy, skeletal joint stiffness, reduced muscle or limb mobility or of the subject, paralysis, a sensory impairment, such as vision or tissue or cell damage. Examples of adverse symptoms occur in particular tissues, or organs, or regions or areas of the body, such as in skin, epidermal or mucosal tissue, gut, gastrointestinal, bowel, genito-urinary tract, pancreas, thymus, lung, liver, kidney, muscle, central or peripheral nerves, spleen, skin, a skeletal joint (e.g., knee, ankle, hip, shoulder, wrist, finger, toe, or elbow), blood or lymphatic vessel, or a cardio-pulmonary tissue or organ. Additional examples of adverse symptoms of an autoimmune response, disorder or disease include T cell production, survival, proliferation, activation or differentiation, and/or production of auto-antibodies, or pro-inflammatory cytokines or chemokines (e.g., TNF-alpha, IL-6, etc.).

Specific non-limiting examples of aberrant or undesirable immune responses, disorders and diseases, inflammatory responses, disorders and diseases, inflammation, autoimmune responses, disorders and diseases, treatable in accordance with the invention include: rheumatoid arthritis, juvenile rheumatoid arthritis, osteoarthritis, psoriatic arthritis, multiple sclerosis (MS), encephalomyelitis, myasthenia gravis, systemic lupus erythematosus (SLE), asthma, allergic asthma, autoimmune thyroiditis, atopic dermatitis, eczematous dermatitis, psoriasis, Sjögren's Syndrome, Crohn's disease, aphthous ulcer, iritis, conjunctivitis, keratoconjunctivitis, ulcerative colitis (UC), inflammatory bowel disease (IBD), cutaneous lupus erythematosus, scleroderma, vaginitis, proctitis, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, interstitial lung fibrosis, Hashimoto's thyroiditis, autoimmune polyglandular syndrome, insulin-dependent diabetes mellitus (IDDM, type I diabetes), insulin-resistant diabetes mellitus (type II diabetes), immune-mediated infertility, autoimmune Addison's disease, pemphigus vulgaris, pemphigus foliaceus, dermatitis herpetiformis, autoimmune alopecia, vitiligo, autoimmune hemolytic anemia, autoimmune thrombocytopenic purpura, pernicious anemia, Guillain-Barre syndrome, stiff-man syndrome, acute rheumatic fever, sympathetic ophthalmia, Goodpasture's syndrome, systemic necrotizing vasculitis, antiphospholipid syndrome or an allergy, Behcet's disease, severe combined immunodeficiency (SCID), recombinase activating gene (RAG 1/2) deficiency, adenosine deaminase (ADA) deficiency, interleukin receptor common γ chain (δ_{c}) deficiency, Janus-associated kinase 3 (JAK3) deficiency and reticular dysgenesis; primary T cell immunodeficiency such as DiGeorge syndrome, Nude syndrome, T cell receptor deficiency, MHC class II deficiency, TAP-2 deficiency (MHC class I deficiency), ZAP70 tyrosine kinase deficiency and purine nucleotide phosphorylase (PNP) deficiency, antibody deficiencies, X-linked agammaglobulinemia (Bruton's tyrosine kinase deficiency), autosomal recessive agammaglobulinemia, Mu heavy chain deficiency, surrogate light chain (γ5/14.1) deficiency, Hyper-IgM syndrome: X-linked (CD40 ligand deficiency) or non-X-linked, Ig heavy chain gene deletion, IgA deficiency, deficiency of IgG subclasses (with or without IgA deficiency), common variable immunodeficiency (CVID), antibody deficiency with normal immunoglobulins; transient hypogammaglobulinemia of infancy, interferon γ receptor (IFNGR1, IFNGR2) deficiency, interleukin 12 or interleukin 12 receptor deficiency, immunodeficiency with thymoma, Wiskott-Aldrich syndrome (WAS protein deficiency), ataxia telangiectasia (ATM deficiency), X-linked lymphoproliferative syndrome (SH2D1A/SAP deficiency), and hyper IgE syndrome.

Specific non-limiting examples of disorders treatable in accordance with the invention methods and uses also include those that affect the skin, or upper or lower respiratory tract, for example, asthma, allergic asthma, bronchiolitis and pleuritis, as well as Airway Obstruction, Apnea, Asbestosis, Atelectasis, Berylliosis, Bronchiectasis, Bronchiolitis, Bronchiolitis Obliterans Organizing Pneumonia, Bronchitis, Bronchopulmonary Dysplasia, Empyema, Pleural Empyema, Pleural Epiglottitis, Hemoptysis, Hypertension, Kartagener Syndrome, Meconium Aspiration, Pleural Effusion, Pleurisy, Pneumonia, Pneumothorax, Respiratory Distress Syndrome, Respiratory Hypersensitivity, Rhinoscleroma, Scimitar Syndrome, Severe Acute Respiratory Syndrome, Silicosis, Tracheal Stenosis, eosinophilic pleural effusions, Histiocytosis; chronic eosinophilic pneumonia; hypersensitivity pneumonitis; Allergic bronchopulmonary aspergillosis; Sarcoidosis; Idiopathic pulmonary fibrosis; pulmonary edema; pulmonary embolism; pulmonary emphysema; Pulmonary Hyperventilation; Pulmonary Alveolar Proteinosis; Chronic Obstructive Pulmonary Disease (COPD); Interstitial Lung Disease; and Topical eosinophilia.

Additional specific non-limiting examples allergies and allergic reactions treatable in accordance with the invention methods and uses also include: Bronchial asthma (extrinsic or intrinsic); Allergic rhinitis; Onchocercal dermatitis; Atopic dermatitis; Allergic conjunctivitis; Drug reactions; Nodules, eosinophilia, rheumatism, dermatitis, and swelling (NERDS); Esophageal and a Gastrointestinal allergy.

Exemplary inhibitors inhibit binding between PKCθ and CD28. Accordingly, inhibitors include any molecule that binds to a PKCθ or a CD28 amino acid sequence, and inhibits binding or interaction between PKCθ and CD28, e.g., binding or interaction between native or endogenous PKCθ and CD28. Accordingly, exemplary inhibitors of binding between PKCθ and CD28 include all PKCθ, CD28 and Lck sequences, subsequences, variants and modified forms, and polymorphisms set forth herein. More specifically, for example, inhibitors include PKCθ amino acid sequence motif MOW, such as by way of example, a ARPPCLPTP or ETRPPCVPTPGK sequence or a subsequence thereof, or a sequence variant of ARPPCLPTP or ETRPPCVPTPGK, including for example ARLPCVPAP, ARLPCVPAS, AKLPHAPAP, AKPPYVPGP or TRLPYLPTP or any other sequence motif set forth in Table 1, or a subsequence or sequence variant thereof, and inhibitors that bind to a PKCθ amino acid sequence motif PXXP, such as by way of example, a ARPPCLPTP or ETRPPCVPTPGK sequence or a subsequence thereof, or a sequence variant of ARPPCLPTP or ETRPPCVPTPGK, including for example ARLPCVPAP, ARLPCVPAS, AKLPHAPAP, AKPPYVPGP or TRLPYLPTP or any other sequence motif set forth in Table 1, or a subsequence or sequence variant thereof (e.g., a substitution of a first or last proline residue with another amino acid such as alanine); and a PYAP motif, for example, spanning amino acids 206-209 of CD28. Such sequences, as set forth herein, can be included within a larger sequence, such as a PKCθ sequence with a length from 9 to about 705 amino acids, where the 9 to about 705 amino acid sequence includes all or portion of a PKCθ amino acid sequence, or does not include all or a portion of a PKCθamino acid sequence. An exemplary PKCθ amino acid sequence is:

MLRLLLALNLFPSIQVTGNKILVKQSPMLVAYDNAVNLSCKYSYNLFSRE
FRASLHKGLDSAVEVCVVYGNYSQQLQVYSKTGFNCDGKLGNESVTFYLQ
NLYVNQTDIYFCKIEVMYPPPYLDNEKSNGTIIHVKGKHLCPSPLFPGPS
KPFWVLVVVGGVLACYSLLVTVAFIIFWVRSKRSRLLHSDYMNMTPRRPG
PTRKHYQPYAPPRDFAAYRS

In addition to the foregoing inhibitors of binding between PKCθ and CD28, additional inhibitors include small molecules. Example, of small molecule inhibitors include organic molecules that bind to PKCθ or CD28, such as in a respective sequence region that includes or consists of a PXXP or PYAP motif.

The term “contacting” means direct or indirect interaction between two or more entities (e.g., between PKCθ, CD28 or Lck and an inhibitor). A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration, or delivery.

In methods and uses of the invention, an inhibitor, such as a PKCθ, CD28 or Lck sequence, can be administered prior to, substantially contemporaneously with or following an undesirable or aberrant immune response, immune disorder, inflammatory response, or inflammation, or an autoimmune response, disorder or disease, GVHD, or host rejection of a cell, tissue or organ transplant (such as heart, liver, lung, bone marrow, etc.), or one or more adverse symptoms, disorders, illnesses, pathologies, diseases, or complications caused by or associated with the foregoing. Thus, methods and uses of the invention may be practiced prior to (i.e. prophylaxis), concurrently with or after evidence of the response, disorder or disease begins, or one or more adverse symptoms, disorders, illnesses, pathologies, diseases, or complications caused by or associated with the undesirable or aberrant immune response, immune disorder, inflammatory response, inflammation or an autoimmune response, disorder or disease, GVHD or host rejection of a cell, tissue or organ transplant (such as heart, liver, lung, bone marrow, etc.). Administering a PKCθ, CD28 or Lck sequence prior to, concurrently with or immediately following development of an adverse symptom may decrease, reduce, inhibit, suppress, limit or control the occurrence, frequency, severity, progression, or duration of one or more adverse symptoms, disorders, illnesses, pathologies, diseases, or complications caused by or associated with the undesirable or aberrant immune response, immune disorder, inflammatory response, inflammation or autoimmune response, disorder or disease, or GVHD, or host rejection of a cell, tissue or organ transplant (such as heart, liver, lung, bone marrow, etc.).

The invention provides combination compositions, methods and uses, such as a PKCθ, CD28 or Lck sequence and a second agent or drug. PKCθ, CD28 or Lck sequence or a composition thereof can be formulated and/or administered in combination with a second agent, drug or treatment, such as an immunosuppressive, anti-inflammatory, or palliative agent, drug or treatment. Accordingly, PKCθ, CD28 or Lck, or a composition thereof can be formulated as a combination and/or administered prior to, substantially contemporaneously with or following administering a second agent, drug or treatment, such as an immunosuppressive, anti-inflammatory, or palliative agent, drug or treatment.

In one embodiment, a composition, method or use includes a PKCθ, CD28 or Lck sequence and an anti-inflammatory agent or drug. Such agents and drugs useful in combinations, methods and uses of the invention include drugs and agents for treatment of an undesirable or aberrant immune response, disorder or disease, an inflammatory response, disorder or disease, inflammation, an autoimmune response, disorder or disease, GVHD, or host rejection of a cell, tissue or organ transplant.

Non-limiting examples of second agents and drugs include anti-inflammatory agents, such as steroidal and non-steroidal anti-inflammatory drugs (NSAIDs) to limit or control inflammatory symptoms. Second agents and drugs also include immunosuppressive corticosteroids (steroid receptor agonists) such as budesonide, prednisone, flunisolide; anti-inflammatory agents such as flunisolide hydrofluoroalkane, estrogen, progesterone, dexamethasone and loteprednol; beta-agonists (e.g., short or long-acting) such as bambuterol, formoterol, salmeterol, albuterol; anticholinergics such as ipratropium bromide, oxitropium bromide, cromolyn and calcium-channel blocking agents; antihistamines such as terfenadine, astemizole, hydroxyzine, chlorpheniramine, tripelennamine, cetirizine, desloratadine, mizolastine, fexofenadine, olopatadine hydrochloride, norastemizole, levocetirizine, levocabastine, azelastine, ebastine and loratadine; antileukotrienes (e.g., anti-cysteinyl leukotrienes (CysLTs)) such as oxatomide, montelukast, zafirlukast and zileuton; phosphodiesterase inhibitors (e.g., PDE4 subtype) such as ibudilast, cilomilast, BAY 19-8004, theophylline (e.g., sustained-release) and other xanthine derivatives (e.g., doxofylline); thromboxane antagonists such as seratrodast, ozagrel hydrochloride and ramatroban; prostaglandin antagonists such as COX-1 and COX-2 inhibitors (e.g., celecoxib and rofecoxib), aspirin; and potassium channel openers. Additional non-limiting examples of classes of other agents and drugs include anti-inflammatory agents that are immunomodulatory therapies, such as pro-inflammatory cytokine antagonists, such as TNFα antagonists (e.g. etanercept, aka Enbrel™) and the anti-IL-6 receptor tocilizumab; immune cell antagonists, such as the B cell depleting agent rituximab and the T cell costimulation blocker abatacept, which have been used to treat rheumatoid arthritis, and antibodies that bind to cytokines, such as anti-IgE (e.g., rhuMAb-E25 omalizumab), and anti-TNFα, IFNγ, IL-1, IL-2, IL-5, IL-6, IL-9, IL-13, IL-16, and growth factors such as granulocyte/macrophage colony-stimulating factor.

As disclosed herein, compositions, methods and uses, such as treatment methods and uses, can provide a detectable or measurable therapeutic benefit or improvement to a subject. A therapeutic benefit or improvement is any measurable or detectable, objective or subjective, transient, temporary, or longer-term benefit to the subject or improvement in the response, disorder or disease, or one or more adverse symptoms, disorders, illnesses, pathologies, diseases, or complications caused by or associated with the undesirable or aberrant response, disorder or disease, etc. Therapeutic benefits and improvements include, but are not limited to, decreasing, reducing, inhibiting, suppressing, limiting or controlling the occurrence, frequency, severity, progression, or duration of an adverse symptom of undesirable or aberrant response, disorder or disease, etc. Therapeutic benefits and improvements also include, but are not limited to, decreasing, reducing, inhibiting, suppressing, limiting or controlling amounts or activity of T cells, auto-antibodies, pro-inflammatory cytokines or chemokines. Compositions, methods and uses of the invention therefore include providing a therapeutic benefit or improvement to a subject.

Compositions, methods and uses of the invention, can be administered in a sufficient or effective amount to a subject in need thereof. An “effective amount” or “sufficient amount” refers to an amount that provides, in single or multiple doses, alone or in combination, with one or more other compositions (therapeutic agents such as a drug), treatments, protocols, or therapeutic regimens agents, a detectable response of any duration of time (long or short term), an expected or desired outcome in or a benefit to a subject of any measurable or detectable degree or for any duration of time (e.g., for minutes, hours, days, months, years, or cured).

The doses of an “effective amount” or “sufficient amount” for treatment (e.g., to ameliorate or to provide a therapeutic benefit or improvement) typically are effective to provide a response, disorder or disease, of one, multiple or all adverse symptoms, consequences or complications of the response, disorder or disease, one or more adverse symptoms, disorders, illnesses, pathologies, diseases, or complications, for example, caused by or associated with an undesirable or an undesirable or aberrant immune response, disorder or disease, an inflammatory response, disorder or disease, inflammation, an autoimmune response, disorder or disease, GVHD, or host rejection of a cell, tissue or organ transplant, to a measurable extent, although decreasing, reducing, inhibiting, suppressing, limiting or controlling progression or worsening of the response, disorder or disease, or GVHD, or host rejection of a cell, tissue or organ transplant, or an adverse symptom thereof, is a satisfactory outcome.

An effective amount or a sufficient amount can but need not be provided in a single administration, may require multiple administrations, and, can but need not be, administered alone or in combination with another composition (e.g., agent), treatment, protocol or therapeutic regimen. For example, the amount may be proportionally increased as indicated by the need of the subject, type, status and severity of the response, disorder, or disease treated or side effects (if any) of treatment. In addition, an effective amount or a sufficient amount need not be effective or sufficient if given in single or multiple doses without a second composition (e.g., another drug or agent), treatment, protocol or therapeutic regimen, since additional doses, amounts or duration above and beyond such doses, or additional compositions (e.g., drugs or agents), treatments, protocols or therapeutic regimens may be included in order to be considered effective or sufficient in a given subject. Amounts considered effective also include amounts that result in a reduction of the use of another treatment, therapeutic regimen or protocol.

An effective amount or a sufficient amount need not be effective in each and every subject treated, prophylactically or therapeutically, nor a majority of treated subjects in a given group or population. An effective amount or a sufficient amount means effectiveness or sufficiency in a particular subject, not a group or the general population. As is typical for such methods, some subjects will exhibit a greater response, or less or no response to a given treatment method or use.

Thus, appropriate amounts will depend upon the condition treated, the therapeutic effect desired, as well as the individual subject (e.g., the bioavailability within the subject, gender, age, etc.).

The term “ameliorate” means a detectable or measurable improvement in a subject's condition or an underlying cellular response. A detectable or measurable improvement includes a subjective or objective decrease, reduction, inhibition, suppression, limit or control in the occurrence, frequency, severity, progression, or duration of the response, disorder or disease, such as an undesirable or undesirable or aberrant immune response, disorder or disease, an inflammatory response, disorder or disease, inflammation, an autoimmune response, disorder or disease, GVHD, or host rejection of a cell, tissue or organ transplant, or one or more adverse symptoms, disorders, illnesses, pathologies, diseases, or complications caused by or associated with the response, disorder or disease, such as an undesirable or aberrant immune response, disorder or disease, an inflammatory response, disorder or disease, inflammation, an autoimmune response, disorder or disease, GVHD, or host rejection of a cell, tissue or organ transplant, or a reversal of the response, disorder or disease, such as an undesirable or aberrant immune response, disorder or disease, an inflammatory response, disorder or disease, inflammation, an autoimmune response, disorder or disease, GVHD, or host rejection of a cell, tissue or organ transplant. Such improvements can also occur at the cellular level.

Thus, a successful treatment outcome can lead to a “therapeutic effect,” or “benefit” of decreasing, reducing, inhibiting, suppressing, limiting, controlling or preventing the occurrence, frequency, severity, progression, or duration of an undesirable or aberrant immune response, disorder or disease, an inflammatory response, disorder or disease, inflammation, an autoimmune response, disorder or disease, GVHD, or host rejection of a cell, tissue or organ transplant, or one or more adverse symptoms or underlying causes or consequences of the undesirable or aberrant immune response, disorder or disease, an inflammatory response, disorder or disease, inflammation, an autoimmune response, disorder or disease, GVHD, or host rejection of a cell, tissue or organ transplant in a subject. Treatment methods affecting one or more underlying causes of the response, disorder or disease or adverse symptom are therefore considered to be beneficial. A decrease or reduction in worsening, such as stabilizing an undesirable or aberrant immune response, disorder or disease, an inflammatory response, disorder or disease, inflammation, an autoimmune response, disorder or disease, GVHD, or host rejection of a cell, tissue or organ transplant, or an adverse symptom thereof, is also a successful treatment outcome.

A therapeutic benefit or improvement therefore need not be complete ablation of the an undesirable or aberrant immune response, disorder or disease, an inflammatory response, disorder or disease, inflammation, an autoimmune response, disorder or disease, GVHD, or host rejection of a cell, tissue or organ transplant, or any one, most or all adverse symptoms, complications, consequences or underlying causes associated with the an undesirable or aberrant immune response, disorder or disease, an inflammatory response, disorder or disease, inflammation, an autoimmune response, disorder or disease, GVHD, or host rejection of a cell, tissue or organ transplant. Thus, a satisfactory endpoint is achieved when there is an incremental improvement in a subject's response, disorder or disease, or a partial decrease, reduction, inhibition, suppression, limit, control or prevention in the occurrence, frequency, severity, progression, or duration, or inhibition or reversal, of the response, disorder or disease (e.g., stabilizing one or more symptoms or complications), such as an undesirable or aberrant immune response, disorder or disease, an inflammatory response, disorder or disease, inflammation, an autoimmune response, disorder or disease, GVHD, or host rejection of a cell, tissue or organ transplant, or one or more adverse symptoms, disorders, illnesses, pathologies, diseases, or complications caused by or associated with an undesirable or aberrant immune response, disorder or disease, an inflammatory response, disorder or disease, inflammation, an autoimmune response, disorder or disease, GVHD, or host rejection of a cell, tissue or organ transplant, over a short or long duration of time (hours, days, weeks, months, etc.).

Effectiveness of a method or use, such as a treatment that provides a potential therapeutic benefit or improvement of a response, disorder or disease, such as an undesirable or aberrant immune response, disorder or disease, an inflammatory response, disorder or disease, inflammation, an autoimmune response, disorder or disease, GVHD, or host rejection of a cell, tissue or organ transplant, can be ascertained by various methods. Such methods include, for example, scores measuring swelling, pain, rash, headache, fever, nausea, diarrhea, bloat, lethargy, skeletal joint stiffness, lack of mobility, rash, or tissue or cell damage. Measuring T cell activation and/or differentiation, cell infiltration of a region, cell accumulation or migration to a region, production of antibodies, cytokines, lymphokines, chemokines, interferons and interleukins, cell growth and maturation factors using various immunological assays, such as ELISA. Determining the degree of cell, tissue or organ damage can be ascertained by CT scanning, MRI, ultrasound, molecular contrast imaging, or molecular ultrasound contrast imaging. For gastrointestinal tract, inflammation can be assessed by endoscopy (colonoscopy, gastroscopy, ERCP), for example. For inflammation of the central nervous system (CNS), cells and cytokines in spinal tap reflect inflammation, for example. CNS inflammation (Multiple sclerosis, Parkinson's, Alzheimer's) may be reflected in the corresponding clinical function scores known in the art, for example. Peripheral nerve inflammation can include functional assessment (motor and sensor), for example.

The term “subject” refers to animals, typically mammalian animals, such as humans, non human primates (e.g., apes, gibbons, chimpanzees, orangutans, macaques), domestic animals (e.g., dogs and cats), farm animals (e.g., horses, cows, goats, sheep, pigs) and experimental animals (e.g., mouse, rat, rabbit, guinea pig). Subjects include animal disease models, for example, animal models of an undesirable or aberrant immune response, disorder or disease, an inflammatory response, disorder or disease, inflammation, an autoimmune response, disorder or disease (e.g., CIA, BXSB, EAE and SCID mice), GVHD, or host rejection of a cell, tissue or organ transplant GVHD and host rejection of a cell, tissue or organ transplant, for in vivo analysis of a composition of the invention.

Subjects appropriate for treatment include those having an undesirable or aberrant immune response, disorder or disease, an inflammatory response, disorder or disease, inflammation, an autoimmune response, disorder or disease, GVHD, or host rejection of a cell, tissue or organ transplant, those undergoing treatment for an undesirable or aberrant immune response, disorder or disease, an inflammatory response, disorder or disease, inflammation, an autoimmune response, disorder or disease, GVHD, or host rejection of a cell, tissue or organ transplant, as well as those who have undergone treatment or therapy for an undesirable or aberrant immune response, disorder or disease, an inflammatory response, disorder or disease, inflammation, an autoimmune response, disorder or disease, GVHD, or host rejection of a cell, tissue or organ transplant, including subjects where theundesirable or aberrant immune response, disorder or disease, inflammatory response, disorder or disease, inflammation, an autoimmune response, disorder or disease, GVHD, or host rejection of a cell, tissue or organ transplant, is in remission.

Subjects also include those that are at increased risk of an undesirable or aberrant immune response, disorder or disease, an inflammatory response, disorder or disease, inflammation, an autoimmune response, disorder or disease, GVHD, or host rejection of a cell, tissue or organ transplant. A candidate subject, for example, has an undesirable or aberrant immune response, disorder or disease, an inflammatory response, disorder or disease, inflammation, an autoimmune response, disorder or disease, GVHD, or host rejection of a cell, tissue or organ transplant, or is being treated with a therapy or drug for an undesirable or aberrant immune response, disorder or disease, an inflammatory response, disorder or disease, inflammation, an autoimmune response, disorder or disease, GVHD, or host rejection of a cell, tissue or organ transplant. Candidate subjects also include subjects that would benefit from or are in need of treatment for an undesirable or aberrant immune response, disorder or disease, an inflammatory response, disorder or disease, inflammation, an autoimmune response, disorder or disease, GVHD, or host rejection of a cell, tissue or organ transplant.

“At risk” subjects typically have increased risk factors for an undesirable or aberrant immune response, disorder or disease, an inflammatory response, disorder or disease, inflammation, an autoimmune response, disorder or disease, GVHD, or host rejection of a cell, tissue or organ transplant. Particular subjects at risk include those that have had an undesirable or aberrant immune response, disorder or disease, an inflammatory response, disorder or disease, inflammation, an autoimmune response, disorder or disease, GVHD, or host rejection of a cell, tissue or organ transplant. Particular subjects at risk also include those prescribed a treatment or therapy for an undesirable or aberrant immune response, disorder or disease, an inflammatory response, disorder or disease, inflammation, an autoimmune response, disorder or disease, GVHD, or host rejection of a cell, tissue or organ transplant. At risk subjects also include those with risk factors include family history (e.g., genetic predisposition), gender, lifestyle (diet, smoking), occupation (medical and clinical personnel, agricultural and livestock workers), environmental factors (allergen exposure), etc.

As set forth herein, PKCθ, CD28 and Lck sequences and compositions thereof may be contacted or provided in vitro, ex vivo or administered or delivered in vivo in various doses and amounts, and frequencies. For example, a PKCθ, CD28 or Lck sequence or a composition thereof can be administered or delivered to provide the intended effect, as a single or as multiple dosages, for example, in an effective or sufficient amount. Exemplary doses range from about 25-250, 250-500, 500-1000, 1000-2500, 2500-5000, 5000-25,000, or 5000-50,000 pg/kg; from about 50-500, 500-5000, 5000-25,000 or 25,000-50,000 ng/kg; from about 50-500, 500-5000, 5000-25,000 or 25,000-50,000 μg/kg; and from about 25-250, 250-500, 500-1000, 1000-2500, 2500-5000, 5000-25,000, or 5000-50,000 mg/kg, on consecutive days, alternating days or intermittently.

Single or multiple (e.g., 2, 3, 4, 5, 6, 7, 8, 9, 10, or more times) administrations or doses can be administered on the same or consecutive days, alternating days or intermittently. For example, a PKCθ, CD28 or Lck sequence or a composition thereof can be administered one, two, three, four or more times daily, on alternating days, bi-weekly, weekly, monthly, bi-monthly, or annually. PKCθ, CD28 and Lck sequences and compositions thereof can be administered for any appropriate duration, for example, for period of 1 hour, or less, e.g., 30 minutes or less, 15 minutes or less, 5 minutes or less, or 1 minute or less.

An inhibitor of binding, such as PKCθ, CD28 or Lck sequences and compositions thereof can be administered to a subject and methods and uses may be practiced prior to, substantially contemporaneously with, or within about 1-60 minutes, hours (e.g., within 1, 2, 3, 4, 5, 6, 8, 12, 24 hours), or days of a symptom or onset of an undesirable or aberrant immune response, disorder or disease, an inflammatory response, disorder or disease, inflammation, an autoimmune response, disorder or disease, GVHD, or host rejection of a cell, tissue or organ transplant.

Compounds can be administered and methods and uses may be practiced via systemic, regional or local administration, by any route. For example, PKCθ, CD28 or Lck sequences and compositions thereof may be administered systemically, regionally or locally, via injection, infusion, orally (e.g., ingestion or inhalation), topically, intravenously, intraarterially, intramuscularly, intraperitoneally, intradermally, subcutaneously, intracavity, intracranially, transdermally (topical), parenterally, e.g. transmucosally or intrarectally (enema) catheter, optically. Compositions, method and uses of the invention including pharmaceutical formulations can be administered via a (micro)encapsulated delivery system or packaged into an implant for administration.

Invention compositions, methods and uses include pharmaceutical compositions, which refer to “pharmaceutically acceptable” and “physiologically acceptable” carriers, diluents or excipients. As used herein, the term “pharmaceutically acceptable” and “physiologically acceptable,” when referring to carriers, diluents or excipients includes solvents (aqueous or non-aqueous), detergents, solutions, emulsions, dispersion media, coatings, isotonic and absorption promoting or delaying agents, compatible with pharmaceutical administration and with the other components of the formulation, and can be contained in a tablet (coated or uncoated), capsule (hard or soft), microbead, emulsion, powder, granule, crystal, suspension, syrup or elixir.

In various embodiments, a pharmaceutical composition includes an inhibitor of binding between PKCθ and CD28. In a particular aspect, an inhibitor includes or consists of a PKCθ, CD28 or Lck sequence. In more particular aspects, a PKCθ sequence includes a ARPPCLPTP sequence, a substitution of an amino acid in a ARPPCLPTP sequence (e.g., a first or last proline residue), a sequence motif set forth in Table 1, or a substitution of an amino acid in a sequence motif set forth in Table 1. Such PKCθ sequences typically have a length from 9 to about 705 amino acids, and the 9 to about 705 amino acid sequence includes all or portion of a PKCθ amino acid sequence, or does not include all or a portion of a PKCθ amino acid sequence. In further particular aspects, a PKCθ sequence has a length of about 9-20, 20-30, 30-40, 40-50, 50-75, 75-100, 100-150, 150-200, 200-250, 250-300, 300-350, 350-400, 400-500, 500-600 or 600-700 amino acid residues.

Pharmaceutical compositions can be formulated to be compatible with a particular route of administration. Compositions for parenteral, intradermal, or subcutaneous administration can include a sterile diluent, such as water, saline, fixed oils, polyethylene glycols, glycerine, propylene glycol or other synthetic solvents. The preparation may contain one or more preservatives to prevent microorganism growth (e.g., antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose).

Pharmaceutical compositions for injection include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). The carrier can be a solvent or dispersion medium containing, for example, water, ethanol, polyol (e.g., glycerol, propylene glycol, and polyetheylene glycol), and suitable mixtures thereof. Fluidity can be maintained, for example, by the use of a coating such as lecithin, or by the use of surfactants. Antibacterial and antifungal agents include, for example, parabens, chlorobutanol, phenol, ascorbic acid and thimerosal. Including an agent that delays absorption, for example, aluminum monostearate and gelatin, can prolong absorption of injectable compositions.

For transmucosal or transdermal administration, penetrants appropriate to the barrier to be permeated are used in the formulation. Such penetrants are known in the art, and include, for example, for transmucosal administration, detergents, bile salts, and fusidic acid derivatives. Transmucosal administration can be accomplished through the use of nasal sprays, inhalation devices (e.g., aspirators) or suppositories. For transdermal administration, the active compounds are formulated into ointments, salves, gels, creams or patches.

Additional pharmaceutical formulations and delivery systems are known in the art and are applicable in the methods of the invention (see, e.g., Remington's Pharmaceutical Sciences (1990) 18th ed., Mack Publishing Co., Easton, Pa.; The Merck Index (1996) 12th ed., Merck Publishing Group, Whitehouse, N.J.; Pharmaceutical Principles of Solid Dosage Forms, Technonic Publishing Co., Inc., Lancaster, Pa., (1993); and Poznansky, et al., Drug Delivery Systems, R. L. Juliano, ed., Oxford, N.Y. (1980), pp. 253-315).

The compositions, methods and uses in accordance with the invention, including PKCθ, CD28 and Lck sequences, subsequences, variants and modified forms, polymorphisms, treatments, therapies, combinations, agents, drugs and pharmaceutical formulations can be packaged in dosage unit form for ease of administration and uniformity of dosage. “Dosage unit form” as used herein refers to physically discrete units suited as unitary dosages treatment; each unit contains a quantity of the composition in association with the carrier, excipient, diluent, or vehicle calculated to produce the desired treatment or therapeutic (e.g., beneficial) effect. The unit dosage forms will depend on a variety of factors including, but not necessarily limited to, the particular composition employed, the effect to be achieved, and the pharmacodynamics and pharmacogenomics of the subject to be treated.

The invention provides kits including PKCθ, CD28 or Lck sequences, subsequences, variants and modified forms, polymorphisms, combination compositions and pharmaceutical formulations thereof, packaged into suitable packaging material. Kits can be used in various in vitro, ex vivo and in vivo methods and uses, for example a treatment method or use as disclosed herein.

A kit typically includes a label or packaging insert including a description of the components or instructions for use in vitro, in vivo, or ex vivo, of the components therein. A kit can contain a collection of such components, e.g., a PKCθ, CD28 or Lck sequence, alone, or in combination with another therapeutically useful composition (e.g., an immune modulatory drug).

The term “packaging material” refers to a physical structure housing the components of the kit. The packaging material can maintain the components sterilely, and can be made of material commonly used for such purposes (e.g., paper, corrugated fiber, glass, plastic, foil, ampules, vials, tubes, etc.).

Kits of the invention can include labels or inserts. Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, such as a disk (e.g., hard disk), optical disk such as CD- or DVD-ROM/RAM, DVD, MP3, magnetic tape, or an electrical storage media such as RAM and ROM or hybrids of these such as magnetic/optical storage media, FLASH media or memory type cards.

Labels or inserts can include identifying information of one or more components therein, dose amounts, clinical pharmacology of the active ingredient(s) including mechanism of action, pharmacokinetics and pharmacodynamics. Labels or inserts can include information identifying manufacturer information, lot numbers, manufacturer location and date.

Labels or inserts can include information on a condition, disorder, disease or symptom for which a kit component may be used. Labels or inserts can include instructions for the clinician or for a subject for using one or more of the kit components in a method, treatment protocol or therapeutic regimen. Instructions can include dosage amounts, frequency or duration, and instructions for practicing any of the methods and uses, treatment protocols or therapeutic regimes set forth herein. Exemplary instructions include, instructions for treating an undesirable or aberrant immune response, disorder or disease, an inflammatory response, disorder or disease, inflammation, an autoimmune response, disorder or disease, GVHD, or host rejection of a cell, tissue or organ transplant. Kits of the invention therefore can additionally include labels or instructions for practicing any of the methods and uses of the invention described herein.

Labels or inserts can include information on any benefit that a component may provide, such as a prophylactic or therapeutic benefit. Labels or inserts can include information on potential adverse side effects, such as warnings to the subject or clinician regarding situations where it would not be appropriate to use a particular composition. Adverse side effects could also occur when the subject has, will be or is currently taking one or more other medications that may be incompatible with the composition, or the subject has, will be or is currently undergoing another treatment protocol or therapeutic regimen which would be incompatible with the composition and, therefore, instructions could include information regarding such incompatibilities.

Invention kits can additionally include other components. Each component of the kit can be enclosed within an individual container and all of the various containers can be within a single package. Invention kits can be designed for cold storage. Invention kits can further be designed to contain PKCθ or CD28 sequences, subsequences, variants and modified forms, polymorphisms, or combination compositions or pharmaceutical compositions.

The invention provides cell-free (e.g., in solution, in solid phase) and cell-based (e.g., in vitro or in vivo) methods of screening for, detecting and identifying agents that modulate binding (interaction) between PKCθ and CD28, and methods of screening, detecting and identifying agents that modulate an undesirable or aberrant immune response, disorder or disease, an inflammatory response, disorder or disease, inflammation, an autoimmune response, disorder or disease, GVHD, or host rejection of a cell, tissue or organ transplant. The methods can be performed in solution, in solid phase, in silica, in vitro, in a cell, and in vivo.

In one embodiment, a method of screening for an agent includes contacting PKCθ and CD28 under conditions allowing binding between PKCθ and CD28 in the presence a test agent; and determining if the test agent inhibits or reduces binding between PKCθ and CD28. In another embodiment, a method of identifying an agent includes contacting PKCθ and CD28 under conditions allowing binding between PKCθ and CD28 in the presence a test agent; and determining if the test agent inhibits or reduces binding between PKCθ and CD28. A reduction or inhibition of binding screens for or identifies the test agent as an agent that decreases, reduces or inhibits interaction of PKCθ with CD28.

In a further embodiment, a method of identifying a candidate agent for modulating (e.g., decreasing, reducing, inhibiting, suppressing, limiting or controlling) an undesirable or aberrant immune response, disorder or disease, an inflammatory response, disorder or disease or inflammation, includes contacting PKCθ and CD28 under conditions allowing binding between PKCθ and CD28 in the presence a test agent; and determining if the test agent inhibits or reduces binding between PKCθ and CD28. If a test agent reduces or inhibits binding, the test agent is a candidate agent for decreasing, reducing, inhibiting, suppressing, limiting or controlling an undesirable or aberrant immune response, disorder or disease, an inflammatory response, disorder or disease or inflammation.

In an additional embodiment, a method of identifying a candidate agent for decreasing, reducing, inhibiting, suppressing, limiting or controlling an autoimmune response, disorder or disease, includes contacting PKCθ and CD28 under conditions allowing binding between PKCθ and CD28 in the presence a test agent; and determining if the test agent inhibits or reduces binding between PKCθ and CD28. If the test agent reduces or inhibits binding, the test agent is a candidate agent for decreasing, reducing, inhibiting, suppressing, limiting or controlling an autoimmune response, disorder or disease.

In yet another embodiment, a method of identifying a candidate agent for decreasing, reducing, inhibiting, suppressing, limiting or controlling graft vs. host disease (GVHD), or host rejection of a cell, tissue or organ transplant includes contacting PKCθ and CD28 under conditions allowing binding between PKCθ and CD28 in the presence a test agent; and determining if the test agent inhibits or reduces binding between PKCθ and CD28. If the test agent reduces or inhibits binding, the test agent is a candidate agent for decreasing, reducing, inhibiting, suppressing, limiting or controlling graft vs. host disease (GVHD), or host rejection of a cell, tissue or organ transplant.

The terms “determining,” “assaying” and “measuring” and grammatical variations thereof are used interchangeably herein and refer to either qualitative or quantitative determinations, or both qualitative and quantitative determinations. When the terms are used in reference to measurement or detection, any means of assessing the relative amount, including the various methods set forth herein and known in the art.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All applications, publications, patents and other references, GenBank citations and ATCC citations cited herein are incorporated by reference in their entirety. In case of conflict, the specification, including definitions, will control.

As used herein, the singular forms “a”, “and,” and “the” include plural referents unless the context clearly indicates otherwise. Thus, for example, reference to “a PKCθ sequence” or “a CD28 sequence” or “a Lck sequence” includes a plurality of such PKCθ, CD28 or Lck sequences, subsequences, variants and modified forms, polymorphisms, or combination compositions or pharmaceutical compositions, and reference to “a PKCθ, CD28 or Lck activity or function” can include reference to one or more PKCθ, CD28 or Lck activities or functions, and so forth.

As used herein, numerical values are often presented in a range format throughout this document. The use of a range format is merely for convenience and brevity and should not be construed as an inflexible limitation on the scope of the invention. Accordingly, the use of a range expressly includes all possible subranges, all individual numerical values within that range. Furthermore, all numerical values or numerical ranges include integers within such ranges and fractions of the values or the integers within ranges unless the context clearly indicates otherwise. This construction applies regardless of the breadth of the range and in all contexts throughout this patent document. Thus, for example, reference to a range of 90-100% includes 91-99%, 92-98%, 93-95%, 91-98%, 91-97%, 91-96%, 91-95%, 91-94%, 91-93%, and so forth. Reference to a range of 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth.

In addition, reference to a range of 1-5,000 fold includes 1, 2, 3, 4, 5, 6, 7, 8, 9, 10, 11, 12, 13, 14, 15, 16, 17, 18, 19, 20, fold, etc., as well as 1.1, 1.2, 1.3, 1.4, 1.5, fold, etc., 2.1, 2.2, 2.3, 2.4, 2.5, fold, etc., and any numerical range within such a ranges, such as 1-2, 5-10, 10-50, 50-100, 100-500, 100-1000, 500-1000, 1000-2000, 1000-5000, etc.

As also used herein a series of range formats are used throughout this document. The use of a series of ranges includes combinations of the upper and lower ranges to provide a range. This construction applies regardless of the breadth of the range and in all contexts throughout this patent document. Thus, for example, reference to a series of ranges such as 5 to 10, 10 to 20, 20 to 30, 30, to 50, 50 to 100, 100 to 150, 150 to 200, 200 to 300, or 300 to 400, 400-500, 500-600, or 600-705, includes ranges such as 5-20, 5-30, 5-40, 5-50, 5-75, 5-100, 5-150, 5-171, and 10-30, 10-40, 10-50, 10-75, 10-100, 10-150, 10-171, and 20-40, 20-50, 20-75, 20-100, 20-150, 20-200, 50 to 200, 50 to 300, 50, to 400, 50 to 500, 100 to 300, 100 to 400, 100 to 500, 100 to 600, 200-400, 200-500, 200 to 600, 200 to 700, and so forth.

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include aspects that are not expressly included in the invention are nevertheless disclosed herein.

A number of embodiments of the invention have been described. Nevertheless, it will be understood that various modifications may be made without departing from the spirit and scope of the invention. Accordingly, the following examples are intended to illustrate but not limit the scope of invention described in the claims.

### EXAMPLES

### Example 1

This example includes a description of materials and methods.

### Antibodies (Abs) and Reagents.

Antibodies and reagents were purchased from the following suppliers: Monoclonal antibodies (mAbs) specific for mouse CD3 (Clone 145-2C11), CD28 (clone 37.51), IL-4 (clone 11B11), IFN-γ (clone XMG1.2) were purchased from Bio-X-Cell. Anti-IL-12/IL23 p40 was from Biolegend. Anti-PKCθ Abs were obtained from BD Transduction Laboratories and Cell Signaling Technology. A C-terminus-specific anti-PKCθ Ab (Clone C-18), which crossreacts with PKCδ, was from Santa Cruz Biotechnology. Rabbit polyclonal anti-CD28 Ab was from Santa Cruz. Anti-talin was from Sigma. The cell tracker blue (CMAC), Alexa-647-conjugated anti-mouse Ig Ab and Alexa-555-conjugated anti-rabbit Ig Ab were obtained from Molecular Probes. Recombinant mouse IL-3, IL-4, IL-6, IL-12, stem cell factor (SCF), and TGF β were purchased from PeproTech. Fluorophore conjugated anti-IL-4, anti-IFN-γ, anti-17A, anti-CD69 and anti-CD25 were from BioLegend. Digitonin was purchased from EMD Chemicals. Ova (323-339) and MCC (88-103) peptides were from Genescript.

### Plasmids.

Retroviral plasmids of full-length human PKCθ and PKCδ were generated via PCR amplification and subcloned into the pMIG retroviral vector (Becart, S. et al. Immunity 29, 704-719 (2008)). PKCθ-ΔV3 (deletion of aa 282-379), PKCθ/δV3 (replacement of PKCθ V3 with aa 282-358 of PKCδ) and PKCδ+θPR (insertion of aa 328-336 of human PKCθ between amino acid I³¹² and Y³¹³ of PKCδ) were constructed using overlapping PCR. Mutated PKCθ versions i.e., P330/6A, P331/4A and P330/1/4/6A (4P-A), were generated using Quikchange II Site-directed Mutagenesis Kit (Stratagene). The PKCθ V3 expression vector was constructed by in-frame subcloning of amino acid 282-379 of human PKCθ into a pMIG vector containing an N-terminal Myc tag. V3-4PA and V3-ΔPR were generated via site-directed mutagenesis and overlapping PCR, respectively, of pMIG-V3. Vectors encoding PKC-EGFP fusion proteins were generated by PCR and subcloned into the retroviral vector pMX (Yokosuka, T. et al. Immunity 29, 589-601 (2008)). The fluorescent tag was attached to the C-terminus of each PKC protein via a polyglycine linker (LESGGGGSGGGG).

Mice and primary cell cultures.

C57BL/6 (B6) mice were housed and maintained under specific pathogen-free conditions in accordance with the Association for Assessment and Accreditation of Laboratory Animal Care International. PKCθ^{-/-} OT-II TCR-Tg mice were generated by intercrossing OT-II TCR-Tg mice and PKCθ-deficient mice, and their T cells were used as a source of Vβ5-Vα2 Ova-specific CD4⁺ T cells. CD4⁺ T cells were isolated by positive selection with anti-CD4 (L3T4) mAb-coated beads (Miltenyi Biotec), and were cultured in RPMI-1640 medium (Mediatech Inc.) supplemented with 10% heat-inactivated fetal bovine serum, 2 mM glutamine, 1 mM sodium pyruvate, 1 mM MEM nonessential amino acids, and 100 U/ml each of penicillin G and streptomycin (Life Technologies, CA). Differentiation of naïve CD4⁺ T cells into Th1, Th2 or Th17 effector cells using standard cytokine and neutralizing antibody cocktails was performed as described (Becart, S. et al. Immunity 29, 704-719 (2008); Canonigo-Balancio, A. J. et al. J Immunol 183, 7259-7267 (2008)).

### Retroviral Transduction.

Platinum-E packaging cells were plated in a 6-well plate in 2 ml RPMI plus 10% FBS. After 24 h, the cells were transfected with 5 μg retroviral plasmid DNA with TransIT-LT1 transfection reagent (Mirus Bio). After an overnight incubation, the medium was replaced and cultures were maintained for at least another 24 h. The retroviral supernatants were then harvested, filtered, supplemented with 5 μg/ml of polybrene and 200 U/ml of recombinant IL-2, and then used to infect CD4⁺ T cells that have been preactivated with plate-bound anti-CD3 (5 μg/ml), soluble anti-CD28 (5 μg/ml) mAb and recombinant IL-2 (200 U/ml). Plates were centrifuged for 1 h at 2,000 rpm and incubated for at least 4 h at 32° C. and for overnight at 37° C., followed by two additional retroviral infections at daily intervals. After the final round of infection, cells were washed and cultured in RPMI medium containing 10% FBS and recombinant IL-2 (200 U/ml) for another three days before restimulation with anti-CD3 plus -CD28 mAbs.

### Immunoprecipitation and Western Blotting.

Retrovirally transduced CD4⁺ T cells were stimulated with anti-CD3 and anti-CD28 mAbs for 5 min. Cell lysis in 1% digitonin lysis buffer, immunoprecipitation and Western blotting were carried out as described (Yokosuka, T. et al. Immunity 29, 589-601 (2008)).

### Luciferase Reporter Assay.

The CD28 response element (RE/AP)— or NFAT-luciferase (Luc) reporter genes have been described (Coudronniere, N. et al. Proc Natl Acad Sci USA 97, 3394-3399 (2000)). MCC-specific hybridoma T cells (So, T. et al. Proc Natl Acad Sci USA 108, 2903-2908 (2011)) were transfected using Ingenio™ Electroporation Reagent (Mirus Bio) according to the manufacturer's instructions. Transfected cells were stimulated for 6 h with DCEK fibroblasts stably expressing I-E^{k} and B7-1 (Gramaglia, I. et al. J Immunol 165, 3043-3050 (2000)), which were prepulsed with 10 μg/ml of MCC peptide, as a source of APCs. The cells were the lysed and Luc activity was quantitated and normalized to the activity of a cotransfected β-galactosidase (β-Gal) reporter gene.

### Immunofluorescence Microscopy.

Full-length human PKCθ and its derivatives were cloned as a GFP fusion protein in the pMX retroviral vector. Retroviral supernatants were used to infect preactivated CD4⁺ T cells from PKCθ^{-/-} or WT OT-II mice. Transduced cells were rested for an additional three days. On the day of experiment, APC were prepared from WT B6 splenocytes that were depleted of CD4⁺ T cells using anti-CD4 (L3T4) mAb-coated beads. T-depleted APC were stained with 20 μM of cell tracker blue (CMAC) and then pulsed with 5 μM Ova peptide, respectively, for 30 min at 37° C. T cell-APC conjugation was carried out at 37° C. for 20 min before fixation with 4% paraformaldehyde and permeabilization with PBS supplemented with 1% BSA and 0.1% Triton X-100. Immunostaining was carried out with the indicated Abs in PBS supplemented with 1% BSA, 0.3% saponin for 1 h at room temperature. Immunofluorescene images were captured using a Marianas digital fluorescence-microscopy system (Intelligent Imaging Innovations) as described (Becart, S. et al. Immunity 29, 704-719 (2008)).

### Bone Marrow (BM) Chimeras.

BM chimeras were produced in irradiated Rag1^{-/-} mice as previously described (Hundt, M. et al. J. Immunol. 183, 1685-1694 (2009). Briefly, BM cells were flushed from the femurs and tibias of PKCθ^{-/-} mice. Lin⁻ bone marrow cells were selected through the Lineage Cell Depletion column (Miltenyi Biotec, Germany) and cultured for 24 h in DMEM media (Mediatech Inc, WI) containing 10% FBS, 10 ng/ml of IL-3, 20 ng/ml of IL-6 and 50 ng/ml of SCF. Retroviral infections were carried out for 3 consecutive days. Infected cells were then sorted for GFP⁺, and 2×10⁵ cells were intravenously injected into irradiated Rag1^{-/-} mice. Analyses were performed 6-8 weeks post-transfer.

### Adoptive Transfer and Ova-Induced Airway Inflammation.

CD4⁺ T cells from OT-II B6 mice were isolated and cultured with plate-bound anti-CD3 (5 μg/ml), soluble anti-CD28 (2.5 μg/ml) and IL-2 (100 U/ml) under Th1 or Th2 polarizing conditions. The cells were retrovirally infected on days 2-4 and cultured for an additional 3 days. GFP⁺ cells were sorted, and 1×10⁶ cells were injected intravenously into naïve WT B6 mice. One day later, mice received aerosolized Ova (5 mg/ml in 20 ml PBS) for 30 min, once a day for 3 consecutive days, using ultrasonic nebulization. Mice were sacrificed 24 h after the last challenge and assessed for lung inflammation. Collection of BAL fluid and determination of cytokine levels were carried out as described (Salek-Ardakani, S. et al. J Immunol 173, 6440-6447 (2004)).

### Statistical Analysis.

Statistical analyses were performed using one-way-ANOVA with post-hoc Bonfferoni's corrections. Unless otherwise indicated, data represent the mean±SEM, with p<0.05 considered statistically significant.

### Example 2

The example includes data indicating that the V3 domain is required for PKCθ IS localization and downstream signaling.

The amino acid sequences between PKCθ and its closest relative in the PKC family, i.e., PKCδ were compared, and these two PKC isoforms share the highest homology within the family (62% identity and 75% homology). However, PKCδ does not translocate to the IS upon interaction between T cells and APCs (Monks, C. R. et al. Nature 385, 83-86 (1997)). Amino acid sequence alignment revealed a significant divergence between the V3 (hinge) domains of these two isoforms (amino acids ˜291-378 of human PKCθ). The V3 region has not been previously implicated in regulation of PKCθ localization or function, other than its general role as a flexible hinge that allows PKC enzymes to undergo a conformational change from a resting state into an “open” active conformation (Keranen, L. M. et al. J Biol Chem 272, 25959-25967 (1997)).

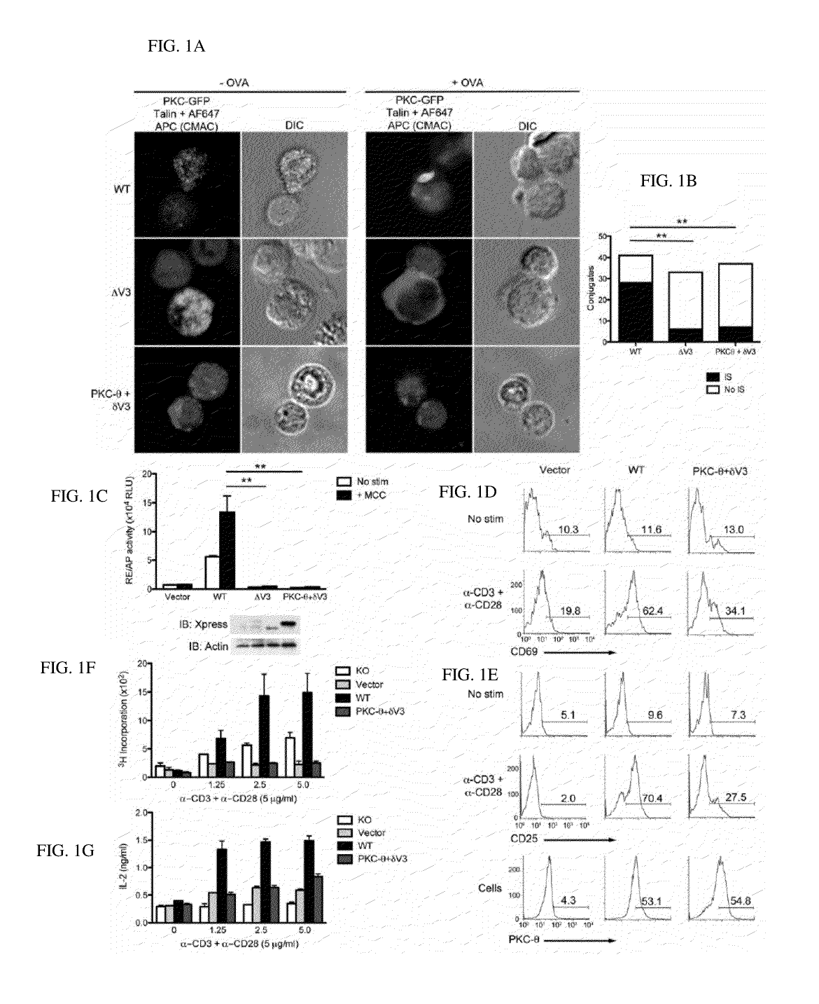

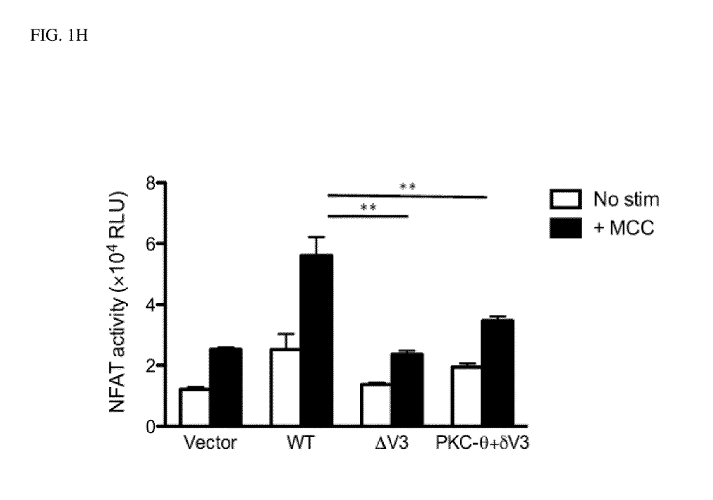

To determine if the V3 domain is required for the IS localization of PKCθ, a V3 deletion mutant (PKCθ-ΔV3) was constructed. When retrovirally transduced into PKCθ^{-/-} TCR-transgenic (Tg) ovalbumin (Ova)-specific OT-II CD4⁺ T cells, wild-type (WT) PKCθ localized in the center of IS, i.e., the cSMAC, following stimulation with Ova peptide-pulsed APCs (FIG. 1a,b), as evident from its central localization relative to that of talin, a known pSMAC marker (Monks, C. R. et al. Nature 395, 82-86 (1998)). In contrast, PKCθ-ΔV3 did not translocate to the IS and, instead, remained largely cytosolic. Since deletion of the V3 domain could cause a gross conformational change that may affect the enzyme's localization, an exchange mutant was generated in which the native V3 domain of PKCθ was replaced with the corresponding domain of PKCδ (PKCθ/δV3). Similar to PKCθ-ΔV3, this mutant also failed to translocate to the IS/cSMAC (FIG. 1a,b). The peripheral IS localization of talin in antigen-stimulated T cells expressing both mutants indicates that the organization of a mature IS was not grossly impaired in the absence of WT PKCθ. These data indicate that the unique V3 domain of PKCθ is required for its selective IS/cSMAC localization.

The quality of T cell activation appears to correlate with the clustering of PKCθ in the IS (Monks, C. R. et al. Nature 395, 82-86 (1998); Huang, J. et al. Eur J Immunol 30, 50-58 (2000)). However, there is no direct evidence that the IS/cSMAC localization of PKCθ is essential for its downstream functions. Since three transcription factors that play important roles in productive T cell activation, i.e., NF-κB, AP-1 and NFAT, are targets of PKCθ (Pfeifhofer, C. et al., J Exp Med 197, 1525-1535 (2003); Coudronniere, N. et al. Proc Natl Acad Sci USA 97, 3394-3399 (2000); Baier-Bitterlich, G. et al. Mol Cell Biol 16, 1842-1850 (1996); Altman, A. et al. Eur J Immunol 34, 2001-2011 (2004); Lin, X. et al. Mol. Cell. Biol. 20, 2933-2940 (2000); Manicassamy, S. et al. J Mol Biol 355, 347-359 (2006)), a study of whether loss or replacement of the PKCθ V3 domain impairs the activation of these transcription factors (FIG. 1c) was undertaken.

Stimulation of empty vector-transfected cells with peptide/APCs resulted consistently in minimal reporter gene stimulation (FIG. 1c, 3d, 4d, and 6c), most likely reflecting the relatively weak stimulus provided by peptide/APC stimulation as compared to the more standard use of saturating anti-CD3/CD28 antibody concentrations in similar reporter assays. T cells transfected with WT PKCθ showed a significant increase in the basal activities of a CD28 response element (RE/AP; FIG. 1c) and NFAT (FIG. 1h), which was further increased by peptide/APC stimulation. However, the activation of these reporter genes was completely abrogated in T cells transfected with PKCθ-ΔV3 or PKCδ/δV3 (FIG. 1c and FIG. 1h).

As an additional analysis of the ability of the PKCθ replacement mutant to activate downstream signaling, the ability to upregulate the expression of CD69 and CD25, two T cell activation markers that have been reported to be regulated by PKCθ (Sun, Z. et al. Nature 404, 402 (2000)) was assessed. Bone marrow (BM) chimeras in irradiated Rag1^{-/-} mice were generated by reconstitution with PKCθ^{-/-} BM cells infected in vitro with bicistronic GFP retroviruses expressing WT PKCθ or PKCθ/δV3, and analyzed the transduced cells 8 weeks later. As expected, anti-CD3/CD28 costimulation of WT PKCθ-reconstituted CD4⁺ T cells greatly upregulated the expression of both CD69 and CD25; however, the ability of PKCθ/δV3 to induce CD69 or CD25 expression was reduced by ˜50-60% (FIG. 1d,e). Both WT PKCθ and PKCθ/δV3 were expressed at similar level in the transduced T cells as revealed by intracellular anti-PKCθ staining (FIG. 1e, bottom panels). Additional functional analysis revealed that, in contrast to WT PKCθ-reconstituted CD4⁺ T cells, which proliferated and produced IL-2 in response to anti-CD3/CD28 stimulation in a dose-dependent manner, the PKCθ/δV3-reconstituted T cells failed to proliferate and produce IL-2 (FIG. 1f,g, respectively) and, in that regard, behaved similarly to CD4⁺ T cells from PKCθ^{-/-} mice or empty vector-reconstituted BM chimera T cells.

Taken together, these data establish that the V3 domain of PKCθ is critical for the activation of PKCθ-dependent TCR/CD28 signaling pathways important for T cell activation. Furthermore, the data shows that this is a non-redundant function that cannot be replaced by the V3 domain of the closely related PKCδ.

### Example 3

This example includes data indicating that the PKCθ V3 domain interacts with CD28.

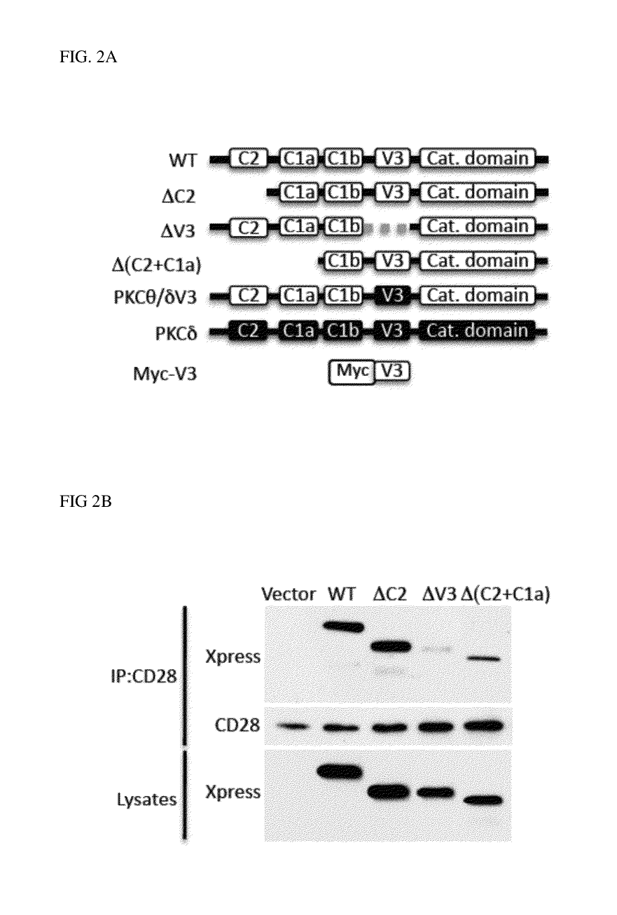

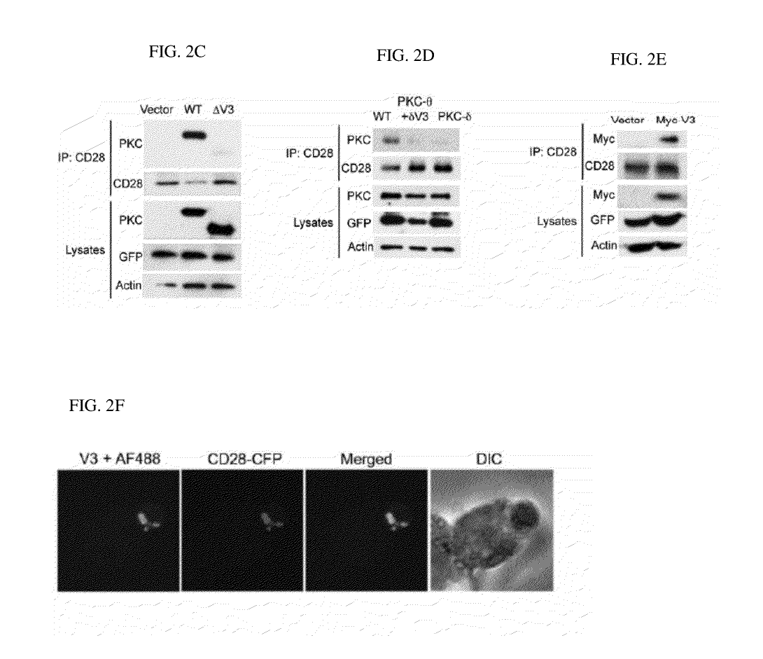

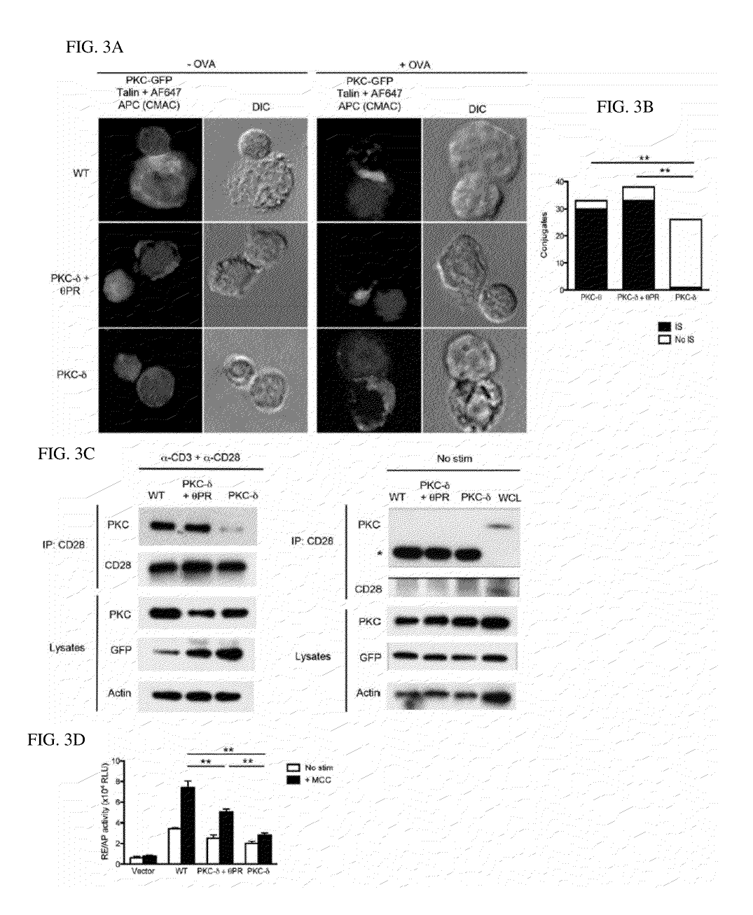

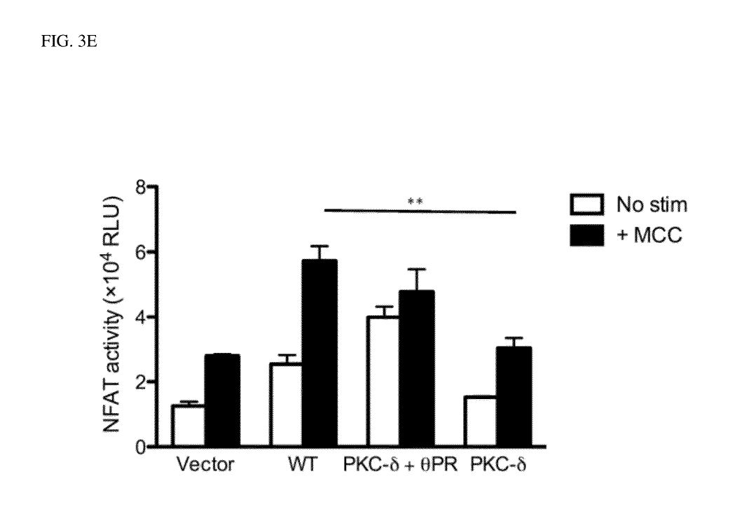

Although not wishing to be bound by theory, one potential role of the PKCθ V3 domain in targeting the enzyme to the IS and rendering it functional reflects a critical association of the V3 domain with a ligand that recruits it to the cSMAC. PKCθ and CD28 have been reported to col-localize in the T cell IS (Tseng, S. Y. et al. J Immunol 175, 7829-7836 (2005); Tseng, S. Y. et al. J Immunol 181, 4852-4863 (2008); Yokosuka T. et al. Immunity 29, 589-601 (2008)) and phorbol ester-induced association between the two (Yokosuka T. et al. Immunity 29, 589-601 (2008)). An analysis of whether anti-CD3/CD28 costimulation will cause PKCθ to associate with CD28 was undertaken. First, Jurkat T cells were transfected with a series of PKCθ deletion mutants (FIG. 2a) and examined potential interactions in CD28 immunoprecipitates (IPs). Upon anti-CD3/CD28 costimulation, WT PKCθ as well as a deletion mutant of the N-terminal C2 domain (ΔC2), previously shown to negatively regulate the activation of PKCθ (Melowic, H. R. et al. J Biol Chem 282, 21467-21476 (2007)), coimmunoprecipitated with CD28 (FIG. 2b). Surprisingly, however, deletion of the V3 domain abolished the interaction between CD28 and PKCθ. When both the C2 and C1a domains of PKCθ (ΔC2+C1a) were deleted, the interaction was reduced, but not abolished. CD28-PKCθ association was strictly dependent on CD3/CD28 coligation since it was not observed in unstimulated cells (FIG. 3c). This analysis was repeated in primary PKCθ^{-/-} CD4⁺ T cells, which were transduced with different PKCθ- or PKCδ-expressing retroviruses. Similar to Jurkat T cells, the ΔV3 mutant did not associate with CD28 (FIG. 2c). In addition, the PKCθ/δV3 mutant as well as WT PKCδ also did not coimmunoprecipitate with CD28 (FIG. 2d). Thus, the V3 domain of PKCθ is necessary for the inducible interaction with CD28.

To determine whether the V3 domain is sufficient for this interaction, PKCθ^{-/-} primary CD4⁺ T cells were infected with a retrovirus expressing a Myc-tagged V3 alone. The V3 domain coimmunoprecipitated with CD28 (FIG. 2e). Therefore, the PKCθ V3 domain is necessary and sufficient for the CD3/CD28-induced interaction of PKCθ with CD28.

### Example 4

This example includes a description of studies of the mapping and functional characterization of a critical PR motif in the PKCθ V3 domain.

To study whether the PKCθ V3 domain contains a unique structural motif that mediates interaction with CD28 and allows it to localize to the IS and mediate its downstream functions, inspection of the V3 domain revealed a PR motif corresponding to amino acid 328-336 of human PKCθ, consisting of the sequence Ala-Arg-Pro-Pro-Cys-Leu-Pro-Thr-Pro (ARPPCLPTP) that was phylogenetically conserved, especially the two internal Pro residues, in PKCθ enzymes from multiple species (Table 1), but absent from the hinge domains of other PKCs. PR motifs have been reported to bind SH3 and WW domains to mediate protein-protein interactions (Kay, B. K. et al. FASEB J 14, 231-241 (2000)).

TABLE 1

| Species | Sequence | Ensembl Accession |
|---|---|---|
| Homo sapiens/Gorilla | ARPPCLPTP | ENST00000263125 |
| gorilla | | |
| Pan troglodytes | ARPPCLPTL | ENSPTRP00000041216 |
| Macaca mulatta | ARPPCLPTP | ENSMMUP00000027347 |
| Canis familiaris | ARLPCVPAP | ENSCAFP00000007725 |
| Felis catus | ARLPCVPAS | ENSFCAP00000008789 |
| Equus caballus | AKLPHAPAP | ENSECAP00000020818 |
| Bos taurus | AKPPYVPGP | ENSBTAT00000060978 |
| Loxodonta africana | TRLPYLPTP | ENSLAFP00000001356 |
| Ailuropoda melanoleuca | AKLPCVPAP | EFB18582.1 (NCBI) |
| Mus musculus | TRPPCVPTP | ENSMUST00000028118 |
| Rattus norvegicus | TRPPCVPTP | ENSRNOP00000025902 |
| Ochotona princeps | TRPPYLPTP | ENSOPRP00000002826 |
| Dipodomys ordii | TRQPNFPTP | ENSDORP00000014980 |
| Spermophilus | ARPPYLPTP | ENSSTOP00000008114 |
| tridecemlineatus | | |
| Tupaia belangeri | ARSPYLPTP | ENSTBEP00000011279 |
| Procavia capensis | TRLPYLPTP | ENSPCAP00000013723 |
| Echinops telfairi | TKLPYLPAP | ENSETEP00000013887 |
| Cavia porcellus | ARLPYLPTG | ENSCPOP00000013395 |
| Dasypus novemcinctus | TRLPYLPVP | ENSDNOP00000008621 |
| Pteropus vampyrus | ARPPHGPAL | ENSPVAP00000014575 |
| Tursiops truncatus | AKLPYGPAP | ENSTTRP00000012525 |
| Xenopus laevis | PKAPGLPMP | BAC79120.1 (NCBI) |
| Danio rerio | AISPLTPAP | ENSDART00000046253 |
| Tetraodon nigroviridis | LLLPNLPLP | CAG04125.1 (NCBI) |
| Takifugu rubripses | VRAPSGPIT | ENSTRUP00000030203 |

Evolutionary conservation of the PxxP motif in the V3 domain of PKCθ. Protein sequences of putative PKCθ enzymes from the indicated organisms were aligned with human PKCθ. The region corresponding to amino acid 328-336 of human PKCθ was extracted and used to generate the consensus sequence with Weblogo. Proline residues that are absolutely conserved in all species are underlined in bold.

The PR motif was analyzed for its importance to the localization and function of PKCθ. The PKCθ PR motif was inserted into the corresponding V3 domain of PKCδ, which is most closely related to PKCθ, and examined whether this altered form of PKCδ (PKCδ+θPR), when expressed in PKCθ^{-/-} CD4⁺ T cells, will localize in the IS. As expected, stimulation of PKCθ^{-/-} OT-II T cells with conjugated Ova-pulsed APCs induced translocation of transduced WT PKCθ and endogenous talin to the cSMAC or the pSMAC, respectively, whereas transduced WT PKCδ did not translocate to the IS, and remained in the cytosol (FIG. 3a,b). However, when the PR motif was introduced into PKCδ, it displayed a similar IS localization to WT PKCθ, suggesting that the PR motif of the PKCθ V3 domain is, indeed, responsible for localization to the IS/cSMAC.

Additional analysis revealed that, similar to WT PKCθ, but unlike WT PKCδ, the altered form of PKCδ, PKCδ+θPR, coimmunopreciptated with CD28 when transduced primary PKCθ^{-/-} CD4⁺ T cells were costimulated with anti-CD3/CD28 antibodies (FIG. 3c, left panel). This association was strictly dependent on T cell stimulation, since it was not observed in similar IPs from unstimulated T cells (FIG. 3c, right panel). Similarly, the PKCδ+θPR mutant was also capable of substantially enhancing the activity of the RE/AP (FIG. 3d) or NFAT (FIG. 3e) reporter genes in stimulated MCC-T hybridoma cells to a degree approaching (˜70-80%) that of WT PKCθ, but significantly higher than that of WT PKCδ. Altogether, these results show that the PR motif in the PKCθ V3 domain is important for the stimulus-dependent association with CD28 and the activation of PKCθ-dependent signaling.

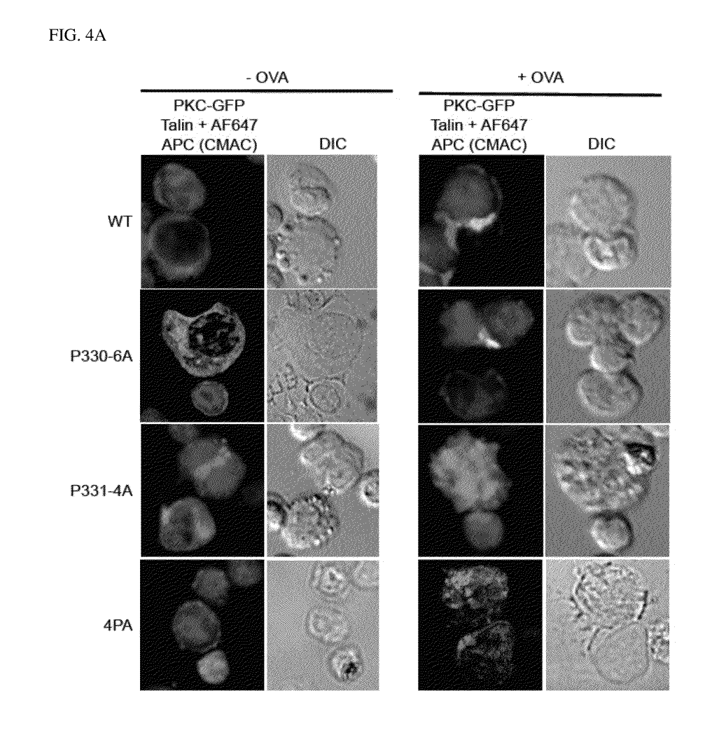

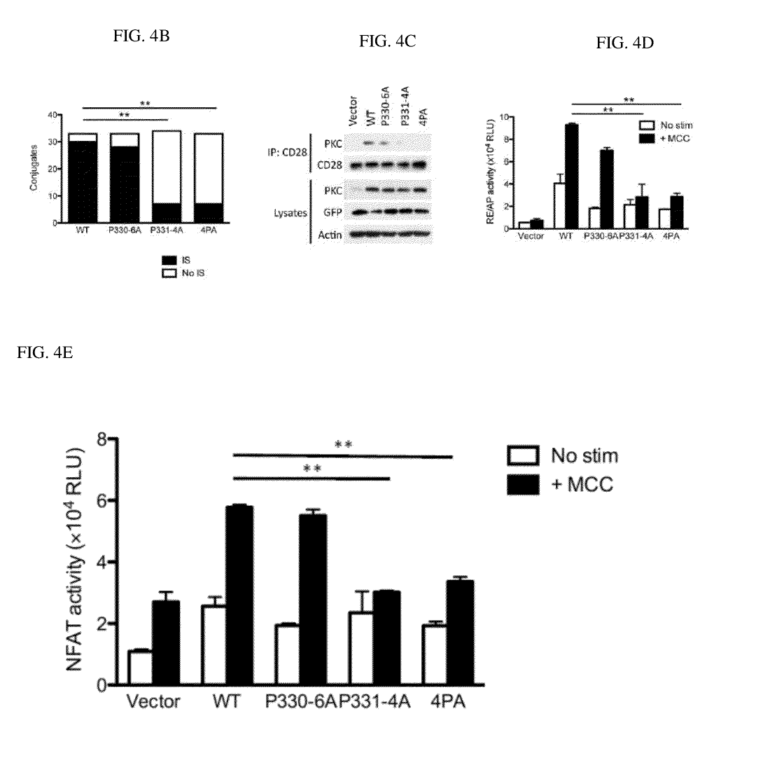

To further delineate which of the four Pro residues in the PR motif play(s) a more important role in the IS localization, CD28 association, and reporter gene activation, a series of point mutations in the PKCθ PR motif (P³³⁰PxxPxP³³⁶; numbers refer to the amino acid residues of the complete human CD28 protein) were engineered, namely, mutants in which the two external Pro residues (P330/6A), the two internal Pro residues (P331/4A), or all four Pro residues (4P-A) were mutated to alanine. As shown in FIG. 4a,b, the IS/cSMAC localization of the transduced P330/6A mutant in Ova-stimulated PKCθ^{-/-} OT-II T cells was intact and similar to that of WT PKCθ. In contrast, the P331/4A and 4P-A mutants failed to localize to the IS and remained largely cytosolic, suggesting that Pro³³¹ and Pro³³⁴ are essential for the antigen-induced recruitment of PKCθ to the IS.

Similar results were obtained when the same PKCθ mutants were analyzed for their ability to co-IP with CD28, and to activate reporter genes in retrovirus-transduced PKCθ^{-/-} T cells. Thus, the P331/4A and the 4P-A mutations, but not the P330/6A mutation, abolished the ability of PKCθ to co-IP with CD28 (FIG. 4c) and greatly reduced activation of either the RE/AP (FIG. 4d) or NFAT (FIG. 4e) reporter genes.

In order to establish the importance of the PxxP motif in a more physiologically relevant setting, anti-CD3/CD28-induced upregulation of CD69 or CD25, as well as the proliferation and IL-2 production by reconstituted CD4⁺ T cells from BM chimeras on a PKCθ^{-/-} background, was analyzed. T cells from mice reconstituted with WT or mutated PKCθ expressed very low levels of CD69 and CD25 on the cell surface in the absence of TCR/CD28 stimulation (FIG. 5a,b, respectively). The surface expression of these activation markers was dramatically elevated in anti-CD3/CD28-stimulated WT PKCθ- or P330/6A-reconstituted T cells. However, when the cells were reconstituted with the P331/4A or 4P-A mutants, expression of CD69 and CD25 was reduced by ˜40-50% (FIG. 5a,b). Similarly, the analysis revealed a marked elevation of proliferation and IL-2 production in CD4⁺ T cells reconstituted with WT PKCθ or PKCθ-P330/6A (FIG. 5c,d) by comparison with the empty vector control-transduced T cells (FIG. 5c,d). CD4⁺ T cells expressing PKCθ-P331/4A or -4P-A mutations displayed defective proliferation (FIG. 5c), and produced some IL-2 only at the two highest concentration of anti-CD3 antibody, albeit at a significantly lower level than WT PKCθ-reconstituted T cells (FIG. 5d).

### Example 5

This example includes a description of studies demonstrating that ectopic expression of the PKCθ V3 domain interferes with T cell activation and differentiation.

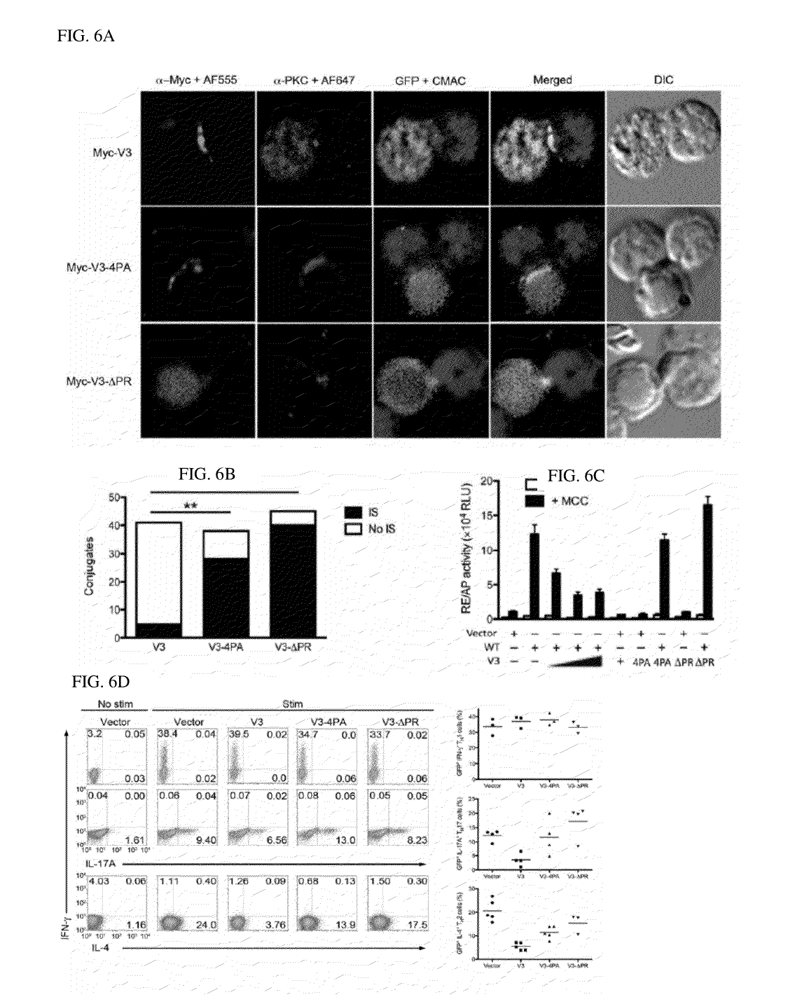

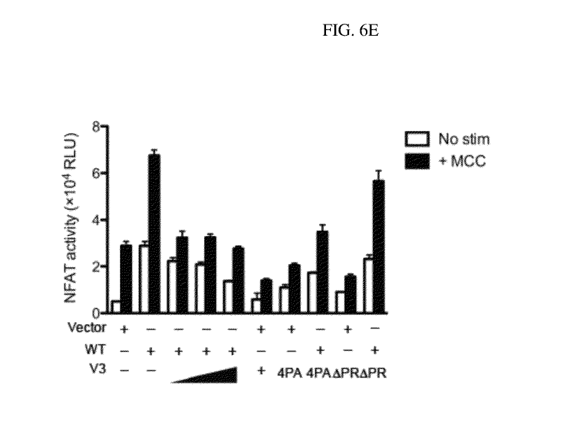

Given the critical role of the PKCθ V3 domain in the enzyme's CD28 association, IS localization, and activation of downstream targets, ectopic expression of the isolated V3 domain was analyzed for interference with the localization and function of PKCθ in stimulated T cells. Presumably V3 domain would function as a “decoy” that will associate with CD28 (FIG. 2e) and, thus, sequester endogenous PKCθ from CD28 and the IS. OT-II TCR-Tg CD4⁺ T cells were infected with a bicistronic GFP retrovirus expressing Myc-tagged V3 alone and examined the subcellular localization of the transduced V3 protein as well as endogenous PKCθ upon conjugation with Ova-loaded APCs. Confocal analysis revealed that the transduced V3 domain alone translocated to and selectively localized in the IS; endogenous PKCθ was sequestered from the IS and found mostly in the cytoplasm of the transduced cells (FIG. 6a,b). However, when the proline-mutated V3 domain (4P-A) or a V3 domain with a deletion of the whole PR motif (ΔPR) were introduced into the T cells, they did not localize anymore in the IS and, furthermore, they did not interfere with the IS localization of endogenous PKCθ following antigen stimulation (FIG. 6a,b).

To determine whether ectopic V3 expression also interfered with PKCθ-dependent TCR/CD28 signaling, the effects of V3 on activation of reporter genes and on T cell differentiation into effector Th cells were analyzed. Expression of WT PKCθ alone significantly increased the antigen-induced activity of both RE/AP (FIG. 6c) and NFAT (FIG. 6e) reporter genes relative to control transfectants. Expression of the V3 alone, on the other hand, did not activate these reporter genes. However, when the V3 domain was coexpressed at increasing levels together with WT PKCθ, it inhibited in a dose-dependent manner the reporter gene activity stimulated by the latter. This inhibitory activity was, however, eliminated when the critical PR motif was mutated or eliminated (FIG. 6c and FIG. 6e), thereby rescuing the WT PKCθ-induced transcription factor activity.

Investigators have reported that the Th2 and Th17 immune responses are compromised in PKCθ^{-/-} mice, whereas the Th1 response remains relatively intact (Marsland, B. J. et al. J Exp Med 200, 181-189 (2004); Salek-Ardakani, S. et al. J Immunol 173, 6440-6447 (2004); Salek-Ardakani, S., et al. J Immunol 175, 7635-7641 (2005)). Therefore, ectopic expression of V3 was analyzed for inhibition of Th differentiation. Preactivated B6 CD4⁺ T cells were infected with retroviruses expressing WT or mutated V3 domains as described above, and cultured in vitro under standard Th1, Th2 or Th17 differentiation conditions. Consistent with findings that PKCθ is dispensable for Th1 responses, differentiation into the Th1 lineage was unaffected by any of the ectopically expressed V3 vectors; in contrast, the non-mutated V3 domain inhibited Th17 and Th2 differentiation by ˜75%, but this inhibition was completely (Th17) or partially (˜60-75%; Th2) reversed when the PR motif was mutated or deleted (FIG. 6d). These results indicate that the V3 domain can act as a decoy to block the specific localization of endogenous PKCθ to the IS and, thus, attenuate its associated signaling and Th differentiation.

### Example 6

This example includes data indicating that PKCθ V3 inhibits Th2-, but not Th1-mediated, airway inflammation.

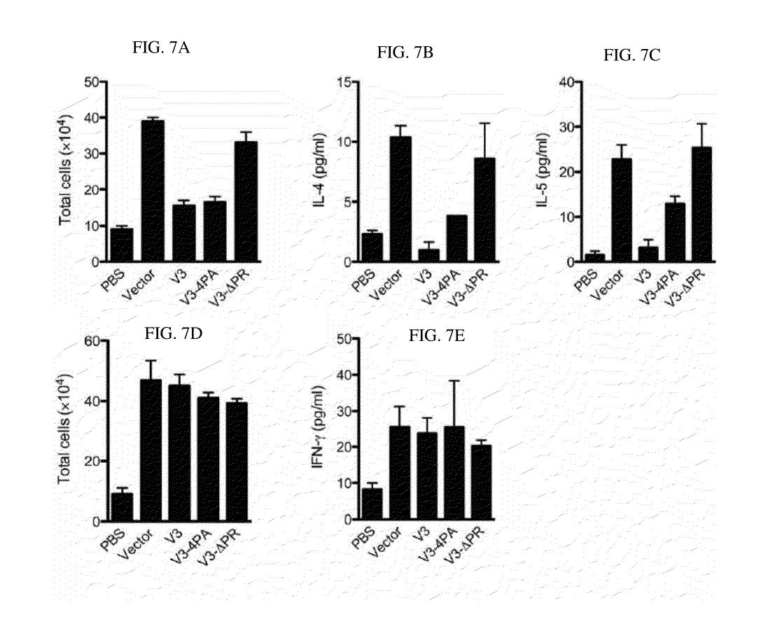

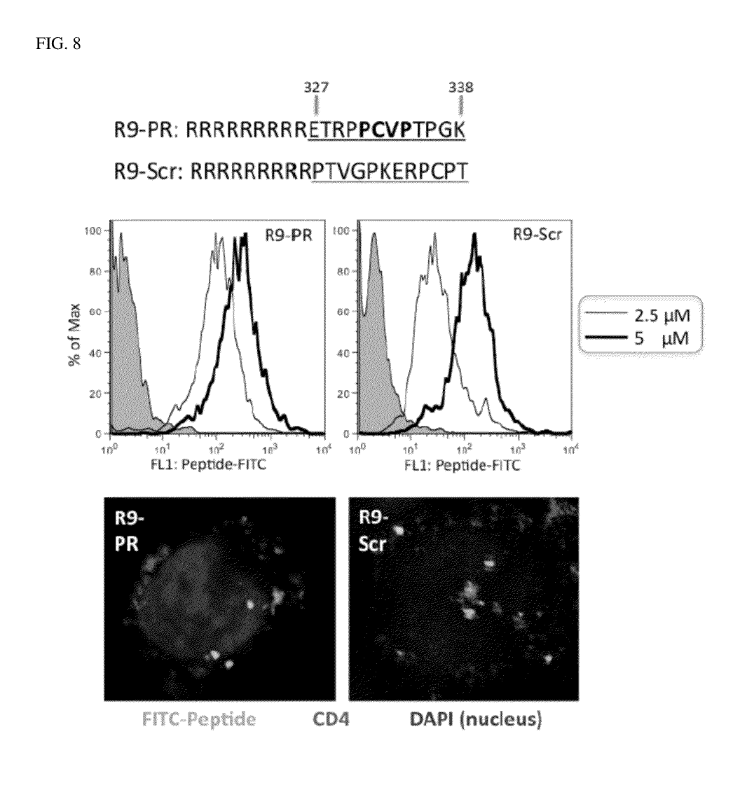

The above findings were extended to an in vivo antigen-specific inflammatory response in an airway inflammation model, using a T cell adoptive transfer system. Mice receiving OT-II Th2 cells transduced with an empty vector developed an inflammatory response by comparison with PBS-challenged control mice, as evidenced by a significant increase in the number of infiltrating leukocytes in the bronchoalveolar lavages (BAL) fluid (FIG. 7a) and in the levels of signature Th2 cytokines, IL-4 (FIG. 7b) and IL-5 (FIG. 7c). Introduction of V3 into the transferred Th2 cells ameliorated the disease by decreasing the levels of infiltrating cells and Th2 cytokines to basal levels (FIG. 7a,b,c). However, expression of the PR motif-deleted V3 domain failed to inhibit the inflammatory response. The 4P-A mutant partially rescued the inhibition, most likely due to the fact that surrounding amino acid residues in addition to the critical Pro residues also contribute to the regulatory function of the V3 domain.

Adoptive transfer of transduced Th1 effector cells similarly induced lung inflammation manifested by leukocyte infiltration (FIG. 7d) and increased IFN-γ levels in the BAL fluid of recipient mice (FIG. 7e). However, in this case the native V3 domain (as well as its mutated variants) did not inhibit the response (FIG. 7d,e), consistent with the Th1-mediated lung inflammation being relatively PKCθ-independent (Marsland, B. et al. J Exp Med 200, 181-189 (2004), Salek-Ardakani, S. et al. J Immunol 173, 6440-6447 (2004); Salek-Ardakani, S. et al. J Immunol 175, 7635-7641 (2005)).

### Example 7

This example shows data indicating that cell-penetrating peptides (CPPs) of the V3 domain of PKCθ are internalized by T cells, and can disrupt interaction of endogenes PKCθ with CD28. Commercial source purified (>95% purity by HPLC) fluoresceinated (FITC) and non-fluoresceinated versions of CPPs corresponding to the PKCθ V3 domain proline-rich (PR) sequence that mediates its activation-induced interaction with CD28 in T cells (R9-PR: RRRRRRRRRETRPPCVPTPGK), or a scrambled version of the same sequence (R9-Scr: RRRRRRRRRPTVGPKERPCPT) were produced. These peptides were characterized for uptake by T cells, and for their ability to disrupt the PKCθ-CD28 interaction in anti-CD3/CD28-costimulated T cells.

In brief, purified splenic CD4⁺ T cells were incubated with 2.5 μM (thin line) or 5 μM (thick line) FITC-R9-PR (left) or FITC-R9-Scr (right) peptides (30 min, 37° C.). FITC fluorescence was analyzed by flow cytometry (FIG. 8). Shaded histogram represents background fluorescence of control, CPP-untreated cells. Of note, the cells were treated with trypsin for 10 minutes to eliminate any FITC-peptide that may be bound to the cells on the outside, under trypsinization conditions that led to complete removal of surface TCR. The data show the FITC-coupled versions of both R9-PR and R9-Scr are taken up by primary CD4⁺ T cells.

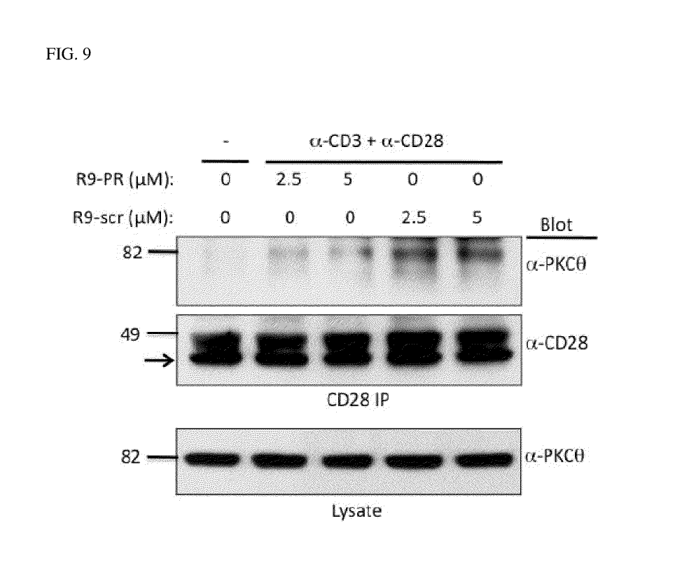

To show that the CPP of the V3 domain can disrupt interaction between PKCθ and CD28, Jurkat cells were incubated with CPPs at the indicated concentrations, as in FIGS. 8 & 9. The cells were left untreated (−) or costimulated with anti-CD3/CD28 (5 min, 37° C.) before cell lysis. PKCθ IPs or lysates were immunoblotted with the indicated antibodies (FIG. 9).

The data demonstrate that R9-PR, but not the control R9-Scr peptide can disrupt the interaction between endogenous PKCθ and CD28 in anti-CD3/CD28-costimulated Jurkat T cells. As disclosed herein, this interaction depends on anti-CD3+CD28 costimulation since it is not observed in unstimulated cells.

Also examined was whether these CPPs are toxic to primary or Jurkat T cells. There was no evidence for any toxicity at CPP concentrations of up to 50 μM and treatment times of up to 2 hours. These findings therefore demonstrate that these CPPs will disrupt the inducible PKCθ-CD28 interaction, which is critical for T cell activation and effective functions of these T cells.

### Example 8

This example includes data showing that expression of the PKCθ V3 domain in primary CD4+ T cells affects Treg differentiation in vitro.

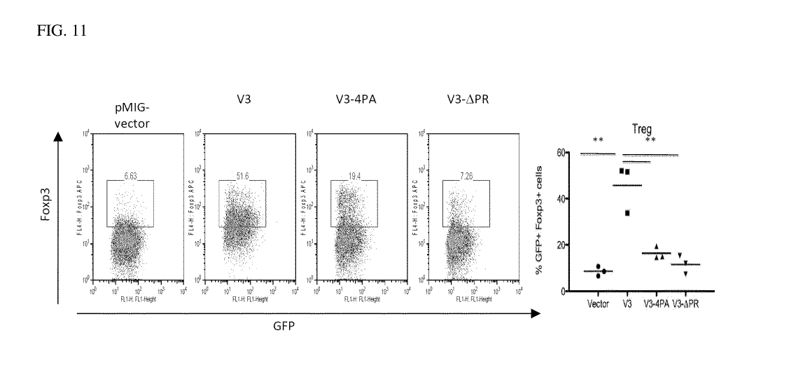

Naïve CD4+ T cells from B6 mice stimulated with anti-CD3 plus anti-CD28 mAbs and differentiated in vitro under Treg-polarizing conditions (IL-2+ TGFβ) were retrovirally transduced with empty pMIG vector, or with the indicated PKCθ V3 vectors. Transduced (GFP+) FoxP3+ cells were analyzed by intracellular staining 8 hours after restimulation. Right panels represent cumulative data showing percentage of GFP+FoxP3+ cells by intracellular staining. The results show that the V3 domain, but not the proline-mutated V3 domain, promotes Treg differentiation by ^{˜}4-fold (FIG. 11). Hence, CPP blocking of PKCθ-CD28 interaction can promote the differentiation of Treg (FoxP3+) cells, which suppress autoimmune diseases.

### Example 9

This example includes data showing that Lck mediates the PKCθ-CD28 interaction

The identification of the PxxP motif, a potential SH3-binding motif, in the PKC-θ V3 domain as being essential for CD28 association was intriguing because mapping analysis of the CD28 cytoplasmic tail revealed that a C-terminal PR motif in murine CD28, i.e. a P²⁶⁰YAP²⁰⁹ motif, was required for the CD28-PKC-θ interaction (data not shown). This is the same CD28 motif required for PKC-θ-CD28 colocalization in the cSMAC, for IL-2 mRNA stabilization, and for lipid raft reorganization, as well as for PKC-θ-dependent T_{H}2- and T_{H}17-mediated inflammatory responses. Since direct association between the PR motifs in PKC-θ and CD28, respectively, is unlikely, it was surmised that this interaction requires an intermediary molecule, with Lck kinase representing a strong candidate. Indeed, it was found that the interaction between CD28 and V3 was absent in Lck-deficient Jurkat (JCam1.6) cells and was restored upon transfection with wild-type Lck, which was also included in the V3-CD28 complex (FIG. 12). The V3 domain associated with SH2-inactivated (R154K) Lck, but CD28 was not present in this complex. When JCam1.6 cells were reconstituted with SH3-inactivated (W97A) Lck, PKC-θ-V3 failed to precipitate both CD28 and Lck (FIG. 12). These findings suggest that Lck mediates the interaction between PKC-θ and CD28, with its SH3 domain binding the PR motif in PKC-θ-V3 and its SH2 domain binding phosphorylated Tyr-207 in the CD28 P²⁰⁶YAP²⁰⁹ motif. This mode of a tri-partite interaction is consistent with findings that the SH2 domain of Lck has a much higher affinity than its SH3 domain for the phosphorylated CD28 PYAP motif and, conversely, that in stimulated T cells, the Lck SH3 domain is considerably more effective than the SH2 domain in binding PKC-θ.

### Example 10

The example includes a discussion of the significance of the foregoing data.

The data indicate that the unique V3 (hinge) domain of PKCθ and, more specifically, a PR motif within this domain, is required for localization via its physical association with CD28 and, consequently, for PKCθ-dependent transcription factor activation, proliferation, cytokine production, and Th2- or Th17-mediated inflammation. This is the first direct evidence that the cSMAC localization of PKCθ and its ability to activate downstream targets are functionally related, both residing within a defined structural motif. The signaling events associated with CD28 costimulation are not entirely understood, and it remains controversial whether CD28 induces unique signals or simply amplifies TCR signals (Acuto, O. et al. Nat Rev Immunol 3, 939-951 (2003); Rudd, C. E. et al. Immunol Rev 229, 12-26 (2009)). Hence, the importance of these findings of a stimulus-dependent association between PKCθ and CD28 stems from the fact that they imply a CD28-specific signaling module that is not shared with TCR signals per se.

Previous reports indicated that efficient PKCθ-mediated transcription factor activation depends on CD28 costimulation (Coudronniere, N. et al. Proc Natl Acad Sci USA 97, 3394-3399 (2000); Bi, K. et al. Nat Immunol 2, 556-563 (2001)). CD28 expression was found to be necessary for the cSMAC localization of PKCθ (Huang, J. et al. Proc Natl Acad Sci USA 99, 9369-9373 (2002)), and this requirement was mapped to a C-terminal P²⁰⁶YAPP motif in murine CD28 (Sanchez-Lockhart, M. et al. J Immunol 181, 7639-7648 (2008)). Several studies reported colocalization of PKCθ and CD28 in IS-resident microclusters (Tseng, S. Y. et al. J Immunol 175, 7829-7836 (2005); Tseng, S. Y. et al. J Immunol 181, 4852-4863 (2008); Yokosuka T. et al. Immunity 29, 589-601 (2008)).

In the mature IS, PKCθ colocalizes with CD28 in a newly defined peripheral subdomain of the cSMAC in a manner dependent on the same P²⁰⁶YAPP motif, and coimmunoprecipitates with CD28 upon phorbol ester stimulation of T cells (Yokosuka, T. et al. Immunity 29 589-601 (2008)). These data disclosed herein establish that this association is induced by physiological stimulation with peptide-pulsed APCs, and is dependent on a PR motif in the V3 domain, which is highly conserved in PKCθ throughout evolution, but is not found in other PKC family members. This unique PR motif defines a novel function of the PKCθ V3 domain, i.e., recruitment of the enzyme to the IS/cSMAC upon TCR/CD28 costimulation. The identification of this PR motif does not exclude potential contribution of other residues in the V3 domain to stable association with CD28 and downstream PKCθ-dependent functions. Indeed, PR mutants of PKCθ were able to partially activate the RE/AP reporter gene (FIG. 4d) and to induce T cell activation (FIG. 5) and, similarly, deletion of the complete PR motif (as opposed to mutating only its four proline residues) impaired more severely the ability of ectopically expressed V3 to inhibit the localization and downstream functions of endogenous PKCθ (FIG. 6).

The importance of the V3 domain in targeting PKCθ to CD28 and the cSMAC and, thereby, rendering it functional, is not inconsistent with the established importance of the C1 domain in targeting PKCθ to the plasma membrane and the IS. The isolated C1 domain of PKCθ was reported to localize in the center of the IS (Spitaler, M. et al. Immunity 24, 535-546 (2006)), although a formal distinction between the cSMAC and pSMAC was not documented. This finding likely reflects high-level accumulation of diacylglycerol (DAG), the PKC-mobilizing second messenger, at the IS. However, this accumulation does not sufficiently explain the unique cSMAC localization of PKCθ since other PKCs that contain a functional DAG-binding C1 domain do not stably localize at the IS. Hence, there must be an additional, PKCθ-specific feature that is responsible for the IS/cSMAC localization of PKCθ. One possible explanation is that the V3 domain, via its CD28 binding, is responsible for this highly selective, sustained and high stoichiometry (Monks, C. R. et al. Nature 385, 83-86 (1997)) localization following the initial binding of the C1 domain to membrane DAG, an event that by itself may be highly dynamic and of low stoichiometry. Indeed, the PKCθ-ΔV3 mutant, which contains an intact C1 domain was mostly localized in the cytosol of Ova/APC-stimulated T cells (FIG. 1a,b).

The stable, long-term recruitment to the cSMAC allows PKCθ to mediate its functions. In contrast, other PKC isoforms that contain a functional DAG-binding C1 domain, e.g., the PKCθ-related PKCδ, which has a similar DAG affinity to that of PKCθ (Melowic, H. R. et al. J Biol Chem 282, 21467-21476 (2007); Stahelin, R. V. et al. J Biol Chem 279, 29501-29512 (2004)), may also localize at DAG-rich membrane sites but will fail to activate sustained signaling and T cell differentiation because of their transient and low stoichiometry recruitment to DAG-rich membrane domains and/or because of substrate specificity distinct from that of PKCθ. In support of this notion, the isolated C1 domain of PKCθ localized only transiently at the IS in stimulated Jurkat T cells, whereas the localization of full-length PKCθ was prolonged and stable (Carrasco, S. et al. Mol Biol Cell 15, 2932-2942 (2004)). Similarly, protein kinase D (PKD), a member of a PKC-related kinase family, which also contains a DAG-binding C1 domain, translocates transiently to the T cell IS (Spitaler, M. Immunity 24, 535-546 (2006)).

In addition to the physical PKCθ-CD28 association disclosed herein, other regulatory events that may contribute to the selective IS/cSMAC localization of PKCθ and its downstream functions include its regulatory tyrosine phosphorylation in the N-terminal C2 domain, which relieves C2-mediated negative regulation (Melowic, H. R. et al. J Biol Chem 282, 21467-21476 (2007); Bi, K. et al. Nat Immunol 2, 556-563 (2001); Liu, Y. et al. J Biol Chem 275, 3603-3609 (2000)), autophosphorylation at Thr-219 in the C1 domain, which was reported to play an important role in the IS localization and function of PKCθ (Thuille, N. et al. EMBO J. 24, 3869-3880 (2005)), and/or specific C1 domain-mediated protein-protein interactions (Colon-Gonzales, F. et al. Biochim Biophys Acta 1761, 827-837 (2006)).

As disclosed herein, the PKCθ PxxP motif is necessary and sufficient to interact with CD28. The identification of this potential SH3-binding motif is somewhat intriguing because a C-terminal PR motif in murine CD28, i.e., a P²⁰⁶ YAP²⁰⁹ motif was required for the interaction with the V3 domain of PKCθ. Interestingly, this is the same motif that is also critical for colocalization of PKCθ with CD28 in the cSMAC, for IL-2 mRNA stabilization and for lipid raft reorganization (Yokosuka, T. et al. Immunity 29, 589-601 (2008); Dodson, L. F. et al. Mol Cell Biol 29, 3710-3721 (2009); Miller, J. et al. Immunol Res 45, 159-172 (2009); Sanchez-Lockhart, M. et al. J Immunol 173, 7120-7124 (2004)), as well as for Th2- and Th17-mediated inflammatory responses that are reported to be dependent on PKCθ (Marsland, B. et al. J Exp Med 200, 181-189 (2004), Salek-Ardakani, S. et al. J Immunol 173, 6440-6447 (2004); Salek-Ardakani, S. et al. J Immunol 175, 7635-7641 (2005); Anderson, K. et al. Autoimmunity 39, 469-478 (2006); Tan, S. L. et al. J Immunol 176, 2872-2879 (2006); Marsland, B. J. et al. J Immunol 178, 3466-3473 (2007)). Since it is unlikely that the identified PKCθ PR motif binds directly to the CD28 C-terminal PR motif, although not wishing to be bound by any theory, this interaction most likely requires an intermediary molecule. One candidate currently under investigation is Lck tyrosine kinase. Lck can bind phosphorylated Tyr-207 in the CD28 PYAP motif via its SH2 domain (Miller, J. et al. Immunol Res 45, 159-172 (2009); Sadra, A. et al. J Immunol 162, 1966-1973 (1999)) and associate with CD28 via its SH2 or SH3 domains (Hofinger, E. et al. J Immunol 174, 3839-3840 (2005); Holdorf, A. D. et al. J Exp Med 190, 375-384 (1999)), and the SH3 domain of Lck was reported to play an important role in its association with PKCθ following T cell stimulation (Liu, Y. et al. J Biol Chem 275, 3603-3609 (2000)). Other proteins that associate with the CD28 PYAP motif, i.e., Grb2 (which contains two SH3 domains and an SH2 domain) and filamin A (Rudd, C. E. et al. Immunol Rev 229, 12-26 (2009); Holdorf, A. D. et al. J Exp Med 190, 375-384 (1999); Kim, H. H. et al. J Biol Chem 273, 296-301 (1998); Okkenhaug, K. et al. J Biol Chem 273, 21194-21202 (1998); Tavano, R. et al. Nat Cell Biol 8, 1270-1276 (2006)), represent additional potential candidates.

Given the selective role of PKCθ in immune response, particularly its requirement in Th2- and Th17-mediated inflammation and GvH disease, but not in Th1 antiviral or GvL responses, PKCθ is an attractive target for pharmacological intervention in a plethora of diseases. Several reports have described purported small molecule selective inhibitors of the catalytic activity of PKCθ(Boschelli, D. H. Curr Top Med Chem 9, 640-654 (2009); Cywin, C. L. et al., Biorg Med Chem Lett 17, 225-230 (2007); Mosyak, L. et al. Biochem Soc Trans 35, 1027-1031 (2007)), which is critical for the activation of downstream signaling pathways (Altman, A. et al. Eur J Immunol 34, 2001-2011 (2004)). However, the catalytic domains of PKC family members are highly conserved and, furthermore, kinase inhibitors generally lack sufficient specificity and, therefore, can display toxic side effects.

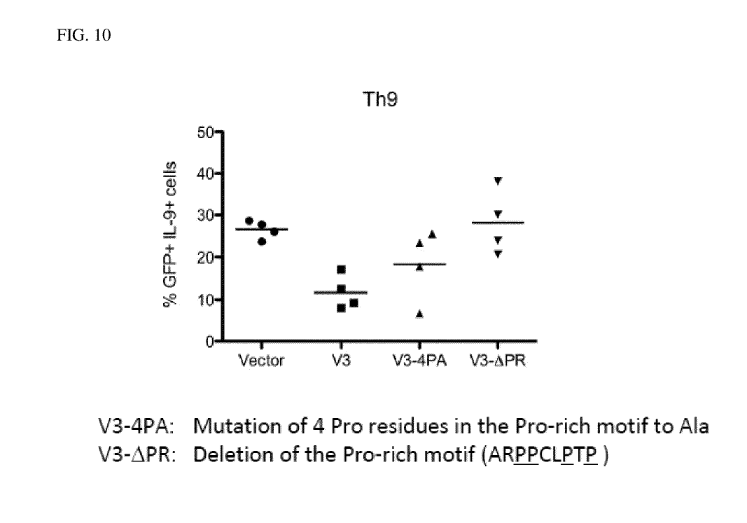

As illustrated in FIG. 10, the V3 domain interferes with PKC-mediated differentiation of Th9 cells. Th9 cells have been reported to play an important role in promoting allergic diseases such as asthma (J. Asthma 48:115, 2011; Curr. Opin. Immunol. 24:1, 2012. These findings indicate that allergic disease such as asthma can be treated in accordance with the invention.

As illustrated in FIG. 11, the V3 domain of PKC promotes differentiation of iTregs (FoxP3+). Treg cells play critical role in preventing and dampening inflammatory and autoimmune responses that are mediated by conventional T cells. Thus, strategies that promote Treg differentiation and/or function may potentially synergize with the inhibition of pathogenic T cells, which require PKC-theta for their disease-promoting function. These findings are consistent with a recent study showing that PKC-theta negatively regulates the function of induced Treg cells (Science 328:372, 2010).

In sum, the invention provides, among other things, methods and uses to attenuate the functions of PKCθ by inhibiting or blocking the obligatory stimulus-induced interaction between the V3 domain of PKCθ and CD28. This inhibition/blockade is the basis for therapeutic agents that can, among others things, selectively suppress, inhibit, reduce or decrease undesired T cell-mediated inflammatory responses (e.g., autoimmunity) while, at the same, preserving desired immunity such as anti-pathogenic responses.

1.-68. (canceled)

69. A method of identifying an inhibitor that inhibits interaction of PKCθ with CD28, comprising:
a) contacting PKCθ with CD28 under conditions allowing binding between PKCθ and CD28 in the presence a test inhibitor; and
b) determining if the test inhibitor inhibits binding between PKCθ and CD28, wherein an inhibition of binding identifies the test inhibitor as an inhibitor that inhibits interaction of PKCθ with CD28.

70. The method of claim 69, wherein the inhibitor binds to a mammalian PKCθ, CD28 or Lck amino acid sequence.

71. The method of claim 69, wherein the inhibitor binds to a PKC9 amino acid sequence comprising ARPPCLPTP, ETRPPCVPTPGK, or a subsequence thereof, or a sequence variant of ARPPCLPTP or ETRPPCVPTPGK or a sequence variant of a subsequence thereof.

72. The method of claim 71, wherein the PKC9 amino acid sequence variant comprises one or more of: ARLPCVPAP, ARLPCVPAS, AKLPHAPAP, AKPPYVPGP, TRLPYLPTP, or any other sequence motif set forth in Table 1.

73. The method of claim 69, wherein the inhibitor comprises a subsequence of full length PKC9 polypeptide or a polymorphism thereof, a subsequence of CD28 comprising a PYAP motif spanning amino acids 206-209 of CD28, or a subsequence of Lck polypeptide or a polymorphism thereof comprising a SH2 and/or SH3 domain of Lck.

74. The method of claim 69, wherein the inhibitor comprises a ARPPCLPTP sequence, a substitution of an amino acid in a ARPPCLPTP sequence, a sequence motif set forth in Table 1, or a substitution of an amino acid in a sequence motif set forth in Table 1, and wherein the sequence has a length from 9 to about 705 amino acids, and wherein the 9 to about 705 amino acid sequence includes all or a portion of a PKC9 amino acid sequence, or does not include all or a portion of a PKC9 amino acid sequence.

75. The method of claim 74, wherein the substitution of an amino acid in a ARPPCLPTP sequence, or a substitution of an amino acid in a sequence motif set forth in Table 1 is in the first or last proline residue.

76. The method of claim 75, wherein the proline residue is substituted with an alanine.

77. The method of claim 69, wherein the inhibitor comprises a small molecule.

78. The method of claim 69, wherein the inhibitor inhibits, decreases or reduces binding of PKC theta to a CD28 cytoplasmic amino acid sequence comprising a PYAP motif spanning amino acids 206-209 of CD28, a subsequence thereof, or a sequence variant thereof.

79. The method of claim 69, wherein the inhibitor does not substantially reduce, inhibit, suppress, decrease, or prevent an anti-pathogenic response of T cells.

80. The method of claim 69, wherein the inhibitor does not substantially reduce, inhibit, suppress, decrease, or prevent one or more desirable responses of T cells.

81. The method of claim 69, wherein the inhibitor does not substantially reduce, inhibit, suppress, decrease, or prevent one or more desirable TLR agonist responses.

82. The method of claim 69, wherein the inhibitor comprises a fusion or a chimera.

83. The method of claim 69, wherein the inhibitor inhibits prevents T cell survival, proliferation, or activation.

84. A method of decreasing an inflammatory response in a subject, comprising administering an inhibitor of binding between protein kinase C (PKC) theta (PKCθ) and CD28 to a subject in an amount to decrease an inflammatory response in the subject.

85. A method of increasing regulatory T cell (Tregs) differentiation or function, comprising administering an inhibitor of binding between protein kinase C (PKC) theta (PKCθ) and CD28 in an amount effective for increasing regulatory T cell differentiation or function.


SEQUENCE LISTING (44 sequences)

SEQ ID NO: 1 - protein, 706 aa, Homo sapiens
MSPFLRIGLSNFDCGSCQSCQGEAVNPYCAVLVKEYVESENGQMYIQKKPTMYPPWDSTFDAHINKGRVMQIIVKGKNVDLISETTVELYSLAERCRKNNGKTEIWLELKPQGRMLMNARYFLEMSDTKDMNEFETEGFFALHQRRGAIKQAKVHHVKCHEFTATFFPQPTFCSVCHEFVWGLNKQGYQCRQCNAAIHKKCIDKVIAKCTGSAINSRETMFHKERFKIDMPHRFKVYNYKSPTFCEHCGTLLWGLARQGLKCDACGMNVHHRCQTKVANLCGINQKLMAEALAMIESTQQARCLRDTEQIFREGPVEIGLPCSIKNEARPPCLPTPGKREPQGISWESPLDEVDKMCHLPEPELNKERPSLQIKLKIEDFILHKMLGKGSFGKVFLAEFKKTNQFFAIKALKKDVVLMDDDVECTMVEKRVLSLAWEHPFLTHMFCTFQTKENLFFVMEYLNGGDLMYHIQSCHKFDLSRATFYAAEIILGLQFLHSKGIVYRDLKLDNILLDKDGHIKIADFGMCKENMLGDAKTNTFCGTPDYIAPEILLGQKYNHSVDWWSFGVLLYEMLIGQSPFHGQDEEELFHSIRMDNPFYPRWLEKEAKDLLVKLFVREPEKRLGVRGDIRQHPLFREINWEELERKEIDPPFRPKVKSPFDCSNFDKEFLNEKPRLSFADRALINSMDQNMFRNFSFMNPGMERLIS

SEQ ID NO: 2 - protein, 706 aa, Gorilla gorilla
MSPFLRIGLSNFDCGSCQSCQGEAVNPYCAVLVKEYVESENGQMYIQKKPTMYPPWDSTFDAHINKGRVMQIIVKGKNVDLISETTVELYSLAERCRKNNGKTEIWLELKPQGRMLMNARYFLEMSDTKDMNEFETEGFFALHQRRGAIKQAKVHHVKCHEFTATFFPQPTFCSVCHEFVWGLNKQGYQCRQCNAAIHKKCIDKVIAKCTGSAINSRETMFHKERFKIDMPHRFKVYNYKSPTFCEHCGTLLWGLARQGLKCDACGMNVHHRCQTKVANLCGINQKLMAEALAMIESTQQARCLRDTEQIFREGPVEIGLPCSIKNEARPPCLPTPGKREPQGISWESPLDEVDKMCHLPEPELNIERPSLQIKLKIEDFILHKMLGKGSFGKVFLAEFKKTNQFFAIKALKKDVVLMDDDVECTMVEKRVLSLAWEHPFLTHMFCTFQTKENLFFVMEYLNGGDLMYHIQSCHKFDLSRATFYAAEIILGLQFLHSKGIVYRDLKLDNILLDKDGHIKIADFGMCKENMLGDAKTNTFCGTPDYIAPEILLGQKYNHSVDWWSFGVLLYEMLIGQSPFHGQDEEELFHSIRMDNPFYPRWLEKEAKDLLVKLFVREPEKRLGVRGDIRQHPLFREINWEELERKEIDPPFRPKVKSPFDCSNFDKEFLNEKPRLSFADRALINSMDQNMFRNFSFMNPGMERLIS

SEQ ID NO: 3 - protein, 706 aa, Pan troglodytes
MSPFLRIGLSNFDCGSCQSCQGEAVNPYCAVLVKEYVESENGQMYIQKKPTMYPPWDSTFDAHINKGRVMQIIVKGKNVDLISETTVELYSLAERCRKNNGKTEIWLELKPQGRMLMNARYFLEMSDTKDMNEFETEGFFALHQRRGAIKQAKVHHVKCHEFTATFFPQPTFCSVCHEFVWGLNKQGYQCRQCNAAIHKKCIDKVIAKCTGSAINSRETMFHKERFKIDMPHRFKVYNYKSPTFCEHCGTLLWGLARQGLKCDACGMNVHHRCQTKVANLCGINQKLMAEALAMIESTQQARCLRDTEQIFREGPVEIGLPCSIKNEARPPCLPTLGKREPQGISWESPLDEVDKMCHLPEPELNIERPSLQIKLKIEDFILHKMLGKGSFGKVFLAEFKKTNQFFAIKALKKDVVLMDDDVECTMVEKRVLSLAWEHPFLTHMFCTFQTKENLFFVMEYLNGGDLMYHIQSCHKFDLSRATFYAAEIILGLQFLHSKGIVYRDLKLDNILLDKDGHIKIADFGMCKENMLGDAKTNTFCGTPDYIAPEILLGQKYNHSVDWWSFGVLLYEMLIGQSPFHGQDEEELFHSIRMDNPFYPRWLEKEAKDLLVKLFVREPEKRLGVRGDIRQHPLFREINWEELERKEIDPPFRPKVKSPFDCSNFDKEFLNEKPRLSFADRALINSMDQNMFRNFSFMNPGMERLIS

SEQ ID NO: 4 - protein, 706 aa, Pongo borneo
MSPFLRIGLSNFDCGSCQSCQGEAVNPYCAVLVKEYVESENGQMYIQKKPTMYPPWDSTFDAHINKGRVMQIIVKGKNVDLISETTVELYSLAERCRKNNGKTEIWLELKPQGRMLMNARYFLEMSDTKDMSEFEMEGFFALHQRRGAIKQAKVHHVKCHEFTATFFPQPTFCSVCHEFVWGLNKQGYQCRQCNAAIHKKCIDKVIAKCTGSAINSRETMFHKERFKIDMPHRFKVYNYKSPTFCEHCGTLLWGLARQGLKCDACGMNVHHRCQTKVANLCGINQKLMAEALAMIESTQQARCLRDTEQIFREGPVEIGLPCSIKNEARPLCLPTPGKREPQGISWESPLDEVDKMCHLPEPELTIERPSLQMKLKIEDFILHKMLGKGSFGKVFLAEFKKTNQFFAIKTLKKDVVLMDDDVECTMVEKRVLSLAWEHPFLTHMFCTFQTKENLFFVMEYLNGGDLMYHIQSCHKFDLSRATFYAAEIILGLQFLHSKGIVYRDLKLDNILLDKDGHIKIADFGMCKENMLGDAKTNTFCGTPDYIAPEILLGQKYNHSVDWWSFGVLLYEMLIGQSPFHGQDEEELFHSIRMDNPFYPRWLEKEAKDLLVKLFVREPEKRLGVRGDIRQHPLFREINWEELERKEIDPPFRPKVKSPYDCSNFDKEFLNEKPRLSFADRALINSMDQNMFRNFSFMNPGMERLIS

SEQ ID NO: 5 - protein, 739 aa, Simia Inuus
MIKHWLSRRGTPKTVPFIAPKQHLSCVVFQGATMSPFLRIGLSNFDCGSCQSCQGEAVNPYCAVLVKEYVESENGQMYIQKKPTMYPPWDSTFDAHINKGRVMQIIVKGKNVDLISETTVELYSLAERCRKNNGKTEIWLELKPQGRMLMNARYFLEMSDTKDMSEFETEGFFALHQRRGAIKQAKVHHVKCHEFTATFFPQPTFCSVCHEFVWGLNKQGYQCRQCNAAIHKKCIDKVIAKCTGSAINSRETMFHKERFKIDMPHRFKVYNYKSPTFCEHCGTLLWGLARQGLKCDACGMNVHHRCQTKVANLCGINQKLMAEALAMIESTQQARCLRDTEQIFREGPVEIGLPCSTKNEARPPCLPTPGKREPQGISWESPLDEVDKMCHLPEPELNKERPSLQMKLKIEDFILHKMLGKGSFGKVFLAEFKKTNQFFAIKALKKDVVLMDDDVECTMVEKRVLSLAWEHPFLTHMFCTFQTKENLFFVMEYLNGGDLMYHIQSCHKFDLSRATFYAAEIILGLQFLHSKGIVYRDLKLDNILLDKDGHIKIADFGMCKENMLGDAKTNTFCGTPDYIAPEILLGQRYNHSVDWWSFGVLLYEMLIGQSPFHGQDEEELFHSIRMDNPFYPRWLEKEAKDLLVKLFVREPEKRLGVRGDIRQHPLFREINWEELERKEIDPPFRPKVKSPYDCSNFDKEFLNEKPRLSFADRALINSMDQNMFRNFSFMNPGMERLIS

SEQ ID NO: 6 - protein, 509 aa, Homo sapiens
MGCGCSSHPEDDWMENIDVCENCHYPIVPLDGKGTLLIRNGSEVRDPLVTYEGSNPPASPLQDNLVIALHSYEPSHDGDLGFEKGEQLRILEQSGEWWKAQSLTTGQEGFIPFNFVAKANSLEPEPWFFKNLSRKDAERQLLAPGNTHGSFLIRESESTAGSFSLSVRDFDQNQGEVVKHYKIRNLDNGGFYISPRITFPGLHELVRHYTNASDGLCTRLSRPCQTQKPQKPWWEDEWEVPRETLKLVERLGAGQFGEVWMGYYNGHTKVAVKSLKQGSMSPDAFLAEANLMKQLQHQRLVRLYAVVTQEPIYIITEYMENGSLVDFLKTPSGIKLTINKLLDMAAQIAEGMAFIEERNYIHRDLRAANILVSDTLSCKIADFGLARLIEDNEYTAREGAKFPIKWTAPEAINYGTFTIKSDVWSFGILLTEIVTHGRIPYPGMTNPEVIQNLERGYRMVRPDNCPEELYQLMRLCWKERPEDRPTFDYLRSVLEDFFTATEGQYQPQP

SEQ ID NO: 7 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 8 - protein, 123 aa, Homo sapiens
MLRLLLALNLFPSIQVTGNKILVKQSPMLVAYDNAVNLSWKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 9 - protein, 101 aa, Homo sapiens
MLRLLLALNLFPSIQVTGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 10 - protein, 9 aa, Homo sapiens
ARPPCLPTP

SEQ ID NO: 11 - protein, 9 aa, Pan troglodytes
ARPPCLPTL

SEQ ID NO: 12 - protein, 9 aa, Macaca mulatto
ARPPCLPTP

SEQ ID NO: 13 - protein, 9 aa, Canis familiaris
ARLPCVPAP

SEQ ID NO: 14 - protein, 9 aa, Felis catus
ARLPCVPAS

SEQ ID NO: 15 - protein, 9 aa, Equus cabal/us
AKLPHAPAP

SEQ ID NO: 16 - protein, 9 aa, Bos taurus
AKPPYVPGP

SEQ ID NO: 17 - protein, 9 aa, Loxodonta africana
TRLPYLPTP

SEQ ID NO: 18 - protein, 9 aa, Ailuropoda melanoleuca
AKLPCVPAP

SEQ ID NO: 19 - protein, 9 aa, Mus musculus
TRPPCVPTP

SEQ ID NO: 20 - protein, 9 aa, Rattus norvegicus
TRPPCVPTP

SEQ ID NO: 21 - protein, 9 aa, Ochotona princeps
TRPPYLPTP

SEQ ID NO: 22 - protein, 9 aa, Dipodomys ordii
TRQPNFPTP

SEQ ID NO: 23 - protein, 9 aa, Spermophilus tridecemlineatus
ARPPYLPTP

SEQ ID NO: 24 - protein, 9 aa, Tupaia belangeri
ARSPYLPTP

SEQ ID NO: 25 - protein, 9 aa, Procavia capensis
TRLPYLPTP

SEQ ID NO: 26 - protein, 9 aa, Echinops telfairi
TKLPYLPAP

SEQ ID NO: 27 - protein, 9 aa, Cavia porcellus
ARLPYLPTG

SEQ ID NO: 28 - protein, 9 aa, Dasypus novemcinctus
TRLPYLPVP

SEQ ID NO: 29 - protein, 9 aa, Pteropus vampyrus
ARPPHGPAL

SEQ ID NO: 30 - protein, 9 aa, Tursiops truncatus
AKLPYGPAP

SEQ ID NO: 31 - protein, 9 aa, Xenopus laevis
PKAPGLPMP

SEQ ID NO: 32 - protein, 9 aa, Danio rerio
AISPLTPAP

SEQ ID NO: 33 - protein, 9 aa, Tetraodon nigroviridis
LLLPNLPLP

SEQ ID NO: 34 - protein, 9 aa, Takifugu rubripses
VRAPSGPIT

SEQ ID NO: 35 - protein, 12 aa, Homo sapiens
ETRPPCVPTPGK

SEQ ID NO: 36 - protein, 12 aa, Artificial Sequence
  Description of Artificial Sequence Polyglycine linker
LESGGGGSGGGG

SEQ ID NO: 37 - protein, 16 aa, Drosophila antennapedia
RQIKIWFQNRRMKWKK

SEQ ID NO: 38 - protein, 9 aa, Artificial Sequence
  Description of Artificial Sequence Cell-penetrating peptide
RRRRRRRRR

SEQ ID NO: 39 - protein, 9 aa, Artificial Sequence
  Description of Artificial Sequence Cell-penetrating peptide
KKKKKKKKK

SEQ ID NO: 40 - protein, 12 aa, Artificial Sequence
  Description of Artificial Sequence Cell-penetrating peptide
RRQRRTSKLMKR

SEQ ID NO: 41 - protein, 27 aa, Artificial Sequence
  Description of Artificial Sequence Cell-penetrating peptide
GWTLNSAGYLLGKINLKALAALAKKIL

SEQ ID NO: 42 - protein, 29 aa, Artificial Sequence
  Description of Artificial Sequence Cell-penetrating peptide
WEAKLAKALAKALAKHLAKALAKALKACE

SEQ ID NO: 43 - protein, 21 aa, Artificial Sequence
  Description of Artificial Sequence Cell-penetrating peptide
RRRRRRRRRETRPPCVPTPGK

SEQ ID NO: 44 - protein, 21 aa, Artificial Sequence
  Description of Artificial Sequence Cell-penetrating peptide
RRRRRRRRRPTVGPKERPCPT